(19) United States
(12) Patent Application Publication
(10) Pub. No.: US 2021/0104297 A1
(43) Pub. Date: Apr. 8, 2021

(54) SYSTEMS AND METHODS FOR DETERMINING TUMOR FRACTION IN CELL-FREE NUCLEIC ACID

(71) Applicant: GRAIL, Inc., Menlo Park, CA (US)

(72) Inventors: Oliver Claude VENN, San Francisco, CA (US); Earl HUBBELL, Palo Alto, CA (US); Onur SAKARYA, Redwood City, CA (US)

(21) Appl. No.: 17/047,676

(22) PCT Filed: Apr. 16, 2019
(86) PCT No.: PCT/US19/27756

§ 371 Date: Oct. 14, 2020

Related U.S. Application Data
(60) Provisional application No. 62/658,479, filed on Apr. 16, 2018.

(51) Int. Cl. G16B 20/20; G16B 40/20; G16B 40/30; G16B 30/10; G16H 50/20; G16H 10/40; G06N 20/00

(52) U.S. Cl. CPC G16B 20/20; G16B 40/20; G16B 40/30; G06N 20/00; G16H 50/20; G16H 10/40; G16B 30/10

(57) ABSTRACT

Systems and methods are disclosed for determining tumor fraction in cell-free nucleic acid of a liquid biological sample of a subject. Sequence reads are obtained using the biological sample. The sequence reads are used to identify support for each variant in a variant set thereby determining an observed frequency of each variant in the variant set. For each respective variant in the variant set, a corresponding reference frequency for the respective variant is obtained in a reference set, where each corresponding reference frequency in the reference set is for a respective variant in an aberrant solid tissue sample obtained from the subject. The observed frequency of each respective variant in the variant set is evaluated against the observed frequency of the respective variant in the reference set thereby determining the tumor fraction in cell-free nucleic acid of the liquid biological sample.

### CROSS REFERENCE TO RELATED APPLICATION

This application claims priority to U.S. Provisional Patent Application No. 62/658,479 entitled “Systems and Methods for Classifying Subjects Using Frequencies of Variants in Cell-Free Nucleic Acid,” filed Apr. 16, 2018, which is hereby incorporated by reference.

### TECHNICAL FIELD

This specification describes determining tumor fraction in cell-free nucleic acid of a subject thereby informing improved classifiers for cancer classification, including the detection of cancer at lower tumor fractions.

### BACKGROUND

The human genome contains about three billion base pairs. Large scale sequencing technologies, such as next generation sequencing (NGS), have afforded the opportunity to achieve sequencing at costs that are less than one U.S. dollar per million bases, and in fact costs of less than ten U.S. cents per million bases have been realized. Such sequencing techniques have enabled the identification of single nucleotide variants (SNVs), small insertion and deletion events (indels), and large-scale copy number variants (CNVs) in aberrant somatic tissues, such as tumor samples.

Such analysis of somatic variants in aberrant somatic tissues provides a basis for understanding the molecular disruptions that underlie the vast differences in individual disease phenotypes or response to treatment. However, the identity of these variants and the frequency of these variants may vary from subject to subject and furthermore may change in any given subject as the disease condition progresses. Moreover, many of the variants associated with diseases such as cancer necessitate deep sequencing of nucleotides from a biological sample such as a tissue biopsy or blood drawn from a subject because of the rarity of some of the variants. For instance, detecting DNA that originated from tumor cells from a blood sample is difficult because circulating tumor DNA (ctDNA) is present at low levels relative to other molecules in cfDNA extracted from the blood. Yet, an understanding of the level of ctDNA in a subject, however low it may be, has the potential to inform treatment decisions as well as improve prognosis and diagnosis.

Given the above background, efficient cost-effective robust techniques for determining ctDNA in a subject that can detect even very low levels of ctDNA are needed in the art.

### SUMMARY

Technical solutions (e.g., computing systems, methods, and non-transitory computer-readable storage mediums) for addressing the above-identified problems with detecting ctDNA are provided.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

One aspect of the present disclosure provides a method of determining tumor fraction in cell-free nucleic acids of a liquid biological sample of a subject. The method comprises, at a computer system having one or more processors, and memory storing one or more programs for execution by the one or more processors, obtaining a first plurality of sequence reads in electronic form from the liquid biological sample of the subject, where the liquid biological sample comprises cell-free nucleic acid molecules. The first plurality of sequence reads is used to identify support for each variant in a first variant set thereby determining an observed frequency of each variant in the first variant set. For each respective variant in the first variant set, a corresponding reference frequency is obtained for the respective variant in a first reference set. Each corresponding reference frequency in the first reference set is for a respective variant in a first aberrant solid tissue sample obtained from the subject. The observed frequency of each respective variant in the first variant set is evaluated against the observed frequency of the respective variant in the first reference set in the first aberrant solid tissue thereby determining a first tumor fraction in cell-free nucleic acid of the liquid biological sample of the subject.

In some embodiments, a variant in the first variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or any aberrant epigenetic modification (e.g., aberrant methylation pattern) associated with a predetermined genomic location.

In some embodiments, a respective sequence read in the first plurality of sequence reads is deemed to support a first variant in the first variant set when the respective sequence read contains all or a portion of the first variant, a respective sequence read in the first plurality of sequence reads is deemed to not support the first variant in the first variant set when the respective sequence read does not contain the first variant, and a number of sequence reads in the first plurality of sequence reads that support the first variant versus a number of sequence reads in the first plurality of sequence reads that do not support the first variant determine the observed frequency of the first variant, which estimates the variant frequency of the first variant within the liquid biological sample.

In some embodiments, the subject is human. In some embodiments, the subject has a cancer from a single primary site of origin. In some embodiments, the subject has a cancer originating from two or more different organs.

In some embodiments, the subject has breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the subject has a predetermined stage of breast cancer, lung cancer, prostate cancer, colorectal cancer, renal cancer, uterine cancer, pancreatic cancer, cancer of the esophagus, head/neck cancer, ovarian cancer, hepatobiliary cancer, cervical cancer, thyroid cancer, bladder cancer, or gastric cancer.

In some embodiments, the first aberrant solid tissue sample is a tumor sample.

In some embodiments, the first variant set consists of a single variant for a single genetic variation at a single locus in the genome of the subject. In alternative embodiments, the first variant set consists of a first variant for a first genetic variation at a first locus in the genome of the subject and a second variant for a second genetic variation at a second locus in the genome of the subject. In still further alternative embodiments, the first variant set consists of a first variant for a first genetic variation at a first locus in the genome of the subject, a second variant for a second genetic variation at a second locus in the genome of the subject, and a third variant for a third genetic variation at a third locus in the genome of the subject.

In some embodiments, the first variant set consists of between two and twenty, consists of between two and 200 variants, comprises 1000 or more variants, or comprises 5000 or more variants and each variant in the first variant set is for a different genetic variation in the genome of the subject.

In some embodiments, the using the sequence reads to identify support for each variant in a variant set comprises aligning a sequence read in the first plurality of sequence reads to a region in a reference genome, or to a lookup table of variants, in order to determine whether the sequence read contains all or a portion of a first variant.

In some embodiments, the using the sequence reads to identify support for each variant in a variant set comprises aligning a sequence read in the first plurality of sequence reads to each entry in a lookup table, where the entry in the lookup table represents a different portion of a genome.

In some embodiments, the subject has stage II, stage III, or stage IV breast cancer and the evaluating the observed frequency of each respective variant in the first variant set against the observed frequency of the respective variant in the first reference set in the first aberrant solid tissue determines that the first tumor fraction of the cell-free nucleic acid is less than 1×10⁻³.

In some embodiments, the method further comprises using the first plurality of sequence reads to identify support for each variant in a second variant set thereby determining an observed frequency of each variant in the second variant set and, for each respective variant in the second variant set, obtaining a corresponding reference frequency for the respective variant in a second reference set, where each corresponding reference frequency in the second reference set is for a respective variant in a second aberrant solid tissue sample obtained from the subject, and evaluating the observed frequency of each respective variant in the second variant set against the observed frequency of the respective variant in the second reference set, thereby determining a second tumor fraction in cell-free nucleic acid of the liquid biological sample of the subject. In some such embodiments, a respective sequence read in the first plurality of sequence reads is deemed to support a variant in the second variant set when the respective sequence read contains all or a portion of the variant, and a respective sequence read in the first plurality of sequence reads is deemed to not support a variant in the second variant set when the respective sequence read does not contain the variant. In some such embodiments, the first aberrant tissue sample consists of a first tumor fraction and the second aberrant tissue sample consists of a second tumor fraction of the same tumor from the subject. In some such embodiments, the first aberrant tissue sample is of a first cancer type and the second aberrant tissue sample is of a second cancer type. In some such embodiments, the first cancer type is the same as the second cancer type. In alternative embodiments, the first cancer type is other than the second cancer type. In some such embodiments, the first cancer type and the second cancer type are each selected from the group consisting of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, and gastric cancer.

In some embodiments, the frequency of each variant in the first reference set is obtained from a second plurality of sequence reads collectively taken from the first aberrant solid tissue sample. In some such embodiments, more than 1000 sequence reads, more than 3000 sequence reads, or more than 5000 sequence reads are collectively taken from the first aberrant solid tissue sample. In some such embodiments, the method further comprises analyzing the second plurality of sequence reads taken from the first aberrant solid tissue sample against a panel of variant candidates. In some such embodiments, the panel of variant candidates comprises between one hundred variants and one thousand variants.

In some embodiments, the second plurality of sequence reads taken from the first aberrant solid tissue sample represents whole genome data for the respective cell. In some embodiments, an average coverage rate of the second plurality of sequence reads taken from the first aberrant solid tissue sample is at least 10×, at least 100×, or at least 2000×.

In some embodiments, the liquid biological sample comprises blood, whole blood, plasma, serum, urine, cerebrospinal fluid, fecal, saliva, sweat, tears, pleural fluid, pericardial fluid, or peritoneal fluid of the subject.

In some embodiments, the biological sample consists of blood, whole blood, plasma, serum, urine, cerebrospinal fluid, fecal, saliva, sweat, tears, pleural fluid, pericardial fluid, or peritoneal fluid of the subject.

In some embodiments, the evaluating the observed frequency of each respective variant in the first variant set to a corresponding reference frequency for the respective variant in the first reference set comprises evaluating a cumulative density function or a cumulative distribution function for the respective variant using the observed frequency and the reference frequency for the respective variant across a range of possible tumor fractions. In some embodiments, a cumulative density function is used and the range is zero percent to 110 percent. In some such embodiments, the first tumor fraction is deemed to be a median value of the cumulative density function.

In some embodiments, a cumulative distribution function is used.

In some embodiments, the cumulative distribution function has the form:

$$P ( x ; p , n ) = {\sum }_{i = 0}^{x} \frac{n !}{i ! ( n - i ) !} {( p )}^{i} {( i - p )}^{( n - i )}$$

where, x=a₂ᵢ, the observed number of sequence reads that support the respective variant in the liquid biological sample, p=t*f₁ᵢ, where t is the estimated first tumor fraction, and f₁ᵢ is the observed frequency of the respective variant in the first variant set, and n=d₂ᵢ, the total number of sequence reads from the biological sample mapping to the genomic location corresponding to the respective variant.

In some embodiments, the cumulative distribution function has the form:

$$log P ( {x}_{k} ; {p}_{k} , {n}_{k} ) = {\sum }_{k} ( {\sum }_{i = 0}^{x} \frac{{n}_{k} !}{i ! ( {n}_{k} - i ) !} {( {p}_{k} )}^{i} {( i - {p}_{k} )}^{( {n}_{k} - i )} )$$

where xₖ=a₂ᵢ, the observed number of sequence reads that support the respective variant k in the liquid biological sample, pₖ=t*f₁ᵢ, where t is the estimated first tumor fraction, and f₁ᵢ, is the observed frequency of the respective variant kin the first variant set, and nₖ=d₂ᵢ, the total number of sequence reads from the biological sample mapping to the genomic location corresponding to the respective variant k.

In some embodiments, the cumulative density function or the cumulative distribution function is drawn under a negative binomial distribution assumption.

In some embodiments, the method further comprises repeating the obtaining of the first plurality of sequence reads at each respective time point in a plurality of time points across an epoch, from a respective biological sample of the subject taken at each respective time point, where the respective liquid biological sample comprises cell-free nucleic acid molecules, thereby obtaining a corresponding first plurality of sequence reads for the subject at each respective time point. Further, in such embodiments, a determination is made of, for each respective time point in the plurality of time points, the support for each variant in the first variant set in the corresponding first plurality of sequence reads for the subject at the respective time point, thereby determining an observed frequency of each respective variant in the first variant set from among the sequence reads in the corresponding first plurality of sequence reads that do support and do not support the respective variant at each time point in the plurality of time points. The observed frequency of each respective variant in the first variant set at each time point in the plurality of time points is evaluated against the observed frequency of the respective variant in the first aberrant solid tissue thereby determining the state or progression of a disease condition in the subject during the epoch in the form of an increase or decrease of the first tumor fraction over the epoch. In some such embodiments, the epoch is a period of months (e.g., less than four months, between one month and four months, etc.) and each time point in the plurality of time points is a different time point in the period of months. In some such embodiments, the epoch is a period of years (between two and ten years) and each time point in the plurality of time points is a different time point in the period of years. In some such embodiments, the epoch is a period of hours (e.g., between one hour and six hours) and each time point in the plurality of time points is a different time point in the period of hours.

In some embodiments, the method further comprises changing a diagnosis of the subject when the first tumor fraction of the subject is observed to change by a threshold amount (e.g., by ten percent, by twenty percent, by thirty percent relative to a reference amount such as at the time of first measurement) across the epoch.

In some embodiments, the method further comprises changing a prognosis of the subject when the first tumor fraction of the subject is observed to change by a threshold amount (e.g., by ten percent, by twenty percent, by thirty percent relative to a reference amount such as at the time of first measurement) across the epoch.

In some embodiments, the method further comprises changing a treatment of the subject when the first tumor fraction of the subject is observed to change by a threshold amount (e.g., by ten percent, by twenty percent, by thirty percent relative to a reference amount such as at the time of first measurement) across the epoch.

In some embodiments, the disease condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof). In some embodiments, the disease condition is a stage of a cancer (e.g., a stage of a breast cancer, a stage of a of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer).

In some embodiments, the disease condition is a predetermined subtype of a cancer.

In some embodiments, the method further comprises applying the first plurality of sequence reads to a trained classifier thereby obtaining a classifier result, where the trained classifier result indicates whether the subject has a first cancer condition, and using the trained classifier result as a basis for diagnosis or prognosis of the subject for the first cancer condition when the first tumor fraction is between 0.003 and 1.0 and the trained classifier result indicates that the subject has the first cancer condition. In some embodiments, the first cancer condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof). In some embodiments, the first cancer condition is a subtype of a cancer (e.g., a subtype of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer).

In some such embodiments, the first tumor fraction is between 0.003 and 1.0 and the first cancer condition is a tissue of origin of a cancer.

In some embodiments, the trained classifier is a neural network, a support vector machine, a decision tree, an unsupervised clustering model, a supervised clustering model, or a regression model.

Another aspect of the present disclosure provides a computing system, comprising one or more processors, memory storing one or more programs to be executed by the one or more processors, the one or more programs comprising instructions for determining tumor fraction in cell-free nucleic acid of a liquid biological sample of a subject by a method comprising obtaining a first plurality of sequence reads in electronic form from the liquid biological sample of the subject, where the liquid biological sample comprises cell-free nucleic acid molecules. The method further comprises using the first plurality of sequence reads to identify support for each variant in a first variant set thereby determining an observed frequency of each variant in the first variant set. The method further comprises, for each respective variant in the first variant set, obtaining a corresponding reference frequency for the respective variant in a first reference set, where each corresponding reference frequency in the first reference set is for a respective variant in a first aberrant solid tissue sample obtained from the subject. The method further comprises evaluating the observed frequency of each respective variant in the first variant set against the observed frequency of the respective variant in the first reference set in the first aberrant solid tissue thereby determining a first tumor fraction in cell-free nucleic acid of the liquid biological sample of the subject.

Another aspect of the present disclosure provides a non-transitory computer readable storage medium storing one or more programs determining tumor fraction in cell-free nucleic acid of a liquid biological sample of a subject. The one or more programs are configured for execution by a computer. The one or more programs comprise instructions for obtaining a first plurality of sequence reads in electronic form from the liquid biological sample of the subject, where the liquid biological sample comprises cell-free nucleic acid molecules. The one or more programs further comprise instructions for using the first plurality of sequence reads to identify support for each variant in a first variant set thereby determining an observed frequency of each variant in the first variant set. The one or more programs comprise instructions that, for each respective variant in the first variant set, obtain a corresponding reference frequency for the respective variant in a first reference set, where each corresponding reference frequency in the first reference set is for a respective variant in a first aberrant solid tissue sample obtained from the subject. The one or more programs comprise instructions for evaluating the observed frequency of each respective variant in the first variant set against the observed frequency of the respective variant in the first reference set in the first aberrant solid tissue thereby determining a first tumor fraction in cell-free nucleic acid of the liquid biological sample of the subject.

Another aspect of the present disclosure provides a method of determining tumor fraction in cell-free nucleic acid of a liquid biological sample of a subject. The method comprises, at a computer system having one or more processors, and memory storing one or more programs for execution by the one or more processors, obtaining a plurality of sequence reads in electronic form from the liquid biological sample of the subject, where the liquid biological sample comprises cell-free nucleic acid molecules. The method further comprises using the plurality of sequence reads to identify support for each variant in a variant set thereby determining an observed frequency of each variant in the first variant set. The method further comprises deeming the observed frequency of the variant having the N^{th} highest allele frequency in the variant set to be the tumor fraction in cell-free nucleic acid of the liquid biological sample of the subject, where N is a positive integer other than one (e.g., 1, 2, 3, 4, 5, etc.).

In some embodiments, a variant in the variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant epigenetic modification pattern (e.g., methylation pattern) associated with a predetermined genomic location.

In some embodiments, a respective sequence read in the plurality of sequence reads is deemed to support a first variant in the variant set when the respective sequence read contains all or a portion of the first variant, and a respective sequence read in the plurality of sequence reads is deemed to not support the first variant in the variant set when the respective sequence read does not contain the first variant, and a number of sequence reads in the plurality of sequence reads that support the first variant versus a number of sequence reads in the plurality of sequence reads that do not support the first variant determine the observed frequency of the first variant, which estimates the variant frequency of the first variant within the liquid biological sample.

In some embodiments, the subject has a cancer from a single primary site of origin. In some embodiments, the subject has a cancer originating from two or more different organs. In some embodiments, the subject has breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the variant set comprises five or more variants, and each respective variant in the variant set is at a different locus in the genome of the subject. In some embodiments, the variant set consists of between three and twenty variants, and each variant in the variant set is for a different genetic variation in the genome of the subject.

In some embodiments, the variant set consists of between 2 and 200 variants, and each variant in the variant set is for a different genetic variation in the genome of the subject. In some embodiments, the variant set comprises 1000 variants, and each variant in the variant set is for a different genetic variation in the genome of the subject.

In some embodiments, the using the plurality of sequence reads to identify support for each variant in a variant set comprises aligning a sequence read in the plurality of sequence reads to a region in a reference genome in order to determine whether the sequence read contains all or a portion of a first variant.

In some embodiments, the using the plurality of sequence reads to identify support for each variant in a variant set comprises aligning a sequence read in the plurality of sequence reads to a lookup table of variants in order to determine whether the sequence read contains all or a portion of a first variant.

In some embodiments, the using the plurality of sequence reads to identify support for each variant in a variant set comprises aligning a sequence read in the plurality of sequence reads to each entry in a lookup table, wherein each entry in the lookup table represents a different portion of a genome.

In some embodiments, the liquid biological sample comprises or consists of blood, whole blood, plasma, serum, urine, cerebrospinal fluid, fecal, saliva, sweat, tears, pleural fluid, pericardial fluid, or peritoneal fluid of the subject.

In some embodiments, the method further comprises repeating the obtaining a plurality of sequence reads at each respective time point in a plurality of time points across an epoch, from a respective biological sample of the subject taken at each respective time point, where the respective biological sample comprises cell-free nucleic acid molecules, thereby obtaining a corresponding plurality of sequence reads for the subject at each respective time point and determining, for each respective time point in the plurality of time points, support for the variant in the variant set that had the N^{th} highest allele frequency in the original deeming step, thereby determining the state or progression of a disease condition in the subject during the epoch in the form of an increase or decrease of the allele frequency of the variant over the epoch.

In some embodiments, the epoch is a period of months (e.g., between 1 month and 4 months) and each time point in the plurality of time points is a different time point in the period of months. In some embodiments, the epoch is a period of years (e.g., between two and ten years) and each time point in the plurality of time points is a different time point in the period of years. In some embodiments, the epoch is a period of hours (e.g., between one hour and six hours) and each time point in the plurality of time points is a different time point in the period of hours.

In some embodiments, the method further comprises changing a diagnosis of the subject when the allele frequency of the variant is observed to change by a threshold amount (e.g., by ten percent, by twenty percent, by thirty percent relative to a reference amount such as at the time of first measurement) across the epoch.

In some embodiments, the method further comprises changing a prognosis of the subject when the allele frequency of the variant is observed to change by a threshold amount (e.g., by ten percent, by twenty percent, by thirty percent relative to a reference amount such as at the time of first measurement) across the epoch.

In some embodiments, the method further comprises changing a treatment of the subject when the allele frequency of the variant is observed to change by a threshold amount (e.g., by ten percent, by twenty percent, by thirty percent relative to a reference amount such as at the time of first measurement) across the epoch.

In some embodiments, the disease condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof). In some embodiments, the disease condition is a stage of cancer (e.g., a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer). In some embodiments, the disease condition is a predetermined subtype of a cancer.

In some embodiments, the method further comprises applying the plurality of sequence reads to a trained classifier thereby obtaining a classifier result, where the trained classifier result indicates whether the subject has a first cancer condition, and using the trained classifier result as a basis for diagnosis of the subject for the first cancer condition when the tumor fraction is between 0.003 and 1.0 and the trained classifier result indicates that the subject has the first cancer condition. In some such embodiments, the first cancer condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof). In some such embodiments, the first cancer condition is a subtype of a cancer (e.g., a subtype of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer). In some such embodiments, the first tumor fraction is between 0.003 and 1.0 and the first cancer condition is a tissue of origin of a cancer. In some embodiments, the trained classifier is a neural network, a support vector machine, a decision tree, an unsupervised clustering model, a supervised clustering model, or a regression model.

Another aspect of the present disclosure provides a computing system, comprising one or more processors, and memory storing one or more programs to be executed by the one or more processors. The one or more programs comprise instructions determining tumor fraction in cell-free nucleic acid of a liquid biological sample of a subject by a method that comprises obtaining a plurality of sequence reads in electronic form from the liquid biological sample of the subject, where the liquid biological sample comprises cell-free nucleic acid molecules. The method further comprises using the plurality of sequence reads to identify support for each variant in a variant set thereby determining an observed frequency of each variant in the first variant set. The method further comprises deeming the observed frequency of the variant having the N^{th} highest allele frequency in the variant set to be the tumor fraction in cell-free nucleic acid of the liquid biological sample of the subject, wherein N is a positive integer other than one.

Another aspect of the present disclosure provides a non-transitory computer readable storage medium storing one or more programs for determining tumor fraction in cell-free nucleic acid of a liquid biological sample of a subject. The one or more programs configured for execution by a computer. The one or more programs comprises instructions for obtaining a plurality of sequence reads in electronic form from the liquid biological sample of the subject, where the liquid biological sample comprises cell-free nucleic acid molecules. The one or more programs further comprise instructions for using the plurality of sequence reads to identify support for each variant in a variant set thereby determining an observed frequency of each variant in the first variant set. The one or more programs further comprise instructions for deeming the observed frequency of the variant having the N^{th} highest allele frequency in the variant set to be the tumor fraction in cell-free nucleic acid of the liquid biological sample of the subject, wherein N is a positive integer other than one.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIGS. 1A and 1B illustrate an example block diagram illustrating a computing device in accordance with some embodiments of the present disclosure.

FIGS. 2A, 2B, 2C, 2D, 2E and 2F illustrate an example flowchart of a method of classifying a subject in accordance with some embodiments of the present disclosure.

FIG. 3 illustrates a box plot in which, for each respective cancer type, the ctDNA fraction of multiple subjects having the respective cancer type is provided, where for each such respective subject the y-axis provides an estimated ctDNA fraction that is based upon a matched pair comparison of the observed frequency of each variant in a variant set from a biological sample (e.g. blood) of the respective subject and the corresponding reference frequency of each such variant obtained from an aberrant tissue sample (e.g., tumor fraction) of the respective subject in accordance with some embodiments of the present disclosure.

FIG. 4 illustrates a plot of the ctDNA fraction of subjects afflicted with any of the cancers illustrated in FIG. 3, as a function of cancer stage in accordance with some embodiments of the present disclosure.

FIG. 5 illustrates a plot of the ctDNA fraction of subjects as a function of breast cancer stage, broken out into three classes, those subjects whose cell free DNA is sufficient to call a variant found in a matching tumor in such subjects without prior knowledge that this variant is in the matching tumor, those subjects whose cell free DNA support a variant that is found in a matching tumor, and those subjects whose cell free DNA do not support a variant that is found in a matching tumor cancers in accordance with some embodiments of the present disclosure.

FIG. 6 illustrates the ability to detect cancer in subjects as a function of their cfDNA fraction in accordance with some embodiments of the present disclosure.

FIGS. 7A and 7B illustrate the ability to call breast cancer as a function of cfDNA fraction, classifier, and breast cancer subtype in accordance with some embodiments of the present disclosure.

FIG. 8 details the precision of the WGBS multi-class classifier for a cohort of subjects spanning the spectrum of different cancers identified in FIG. 3 as a function of ctDNA fraction in accordance with some embodiments of the present disclosure.

FIG. 9 details the percentage of subjects that exhibit a minimum ctDNA fraction as a function of clinical stage in accordance with some embodiments of the present disclosure.

FIG. 10 illustrates the positive association of tumor size with ctDNA fraction, across all stages of cancer in accordance with some embodiments of the present disclosure.

FIG. 11 illustrates the association of ctDNA fraction with the Ki67 marker for proliferation in accordance with some embodiments of the present disclosure.

FIG. 12 illustrates a flowchart of a method for preparing a nucleic acid sample for sequencing in accordance with some embodiments of the present disclosure.

FIG. 13 is a graphical representation of the process for obtaining sequence reads in accordance with some embodiments of the present disclosure.

FIG. 14 is a flowchart of a method for determining variants of sequence reads in accordance with some embodiments of the present disclosure.

FIG. 15 is a flowchart of a method for obtaining a methylation state vector for the purpose of identifying variants in accordance with some embodiments of the present disclosure.

FIG. 16 provides the cumulative density function across a range of trial estimated shedding rates in accordance with some embodiments of the present disclosure.

FIG. 17 illustrates the consistency in tumor fraction measurements made between a tumor matching embodiment and a second highest allele embodiment of the present disclosure.

FIG. 18 illustrates details of a CCGA study that served as a basis for determination of cell free tumor fraction in accordance with some embodiments of the present disclosure.

FIGS. 19A and 19B provide information on sensitivity of models, trained using the training set summarized in FIG. 18, against both the training set (FIG. 19A, N=1,416) and the test set (FIG. 19B, N=847) summarized in FIG. 18, broken out by tumor of origin, in accordance with an embodiment of the present disclosure.

FIGS. 19C and 19D provide information on tumor fraction in the training set (FIG. 19C) and the test set (FIG. 19D) summarized in FIG. 18 broken out by tumor of origin in accordance with an embodiment of the present disclosure.

FIGS. 20A and 20B illustrate cfDNA tumor fraction as calculated by comparing cfDNA WGS with tumor WGS results by stage for breast cancer, colorectal cancer, lung cancer, and other cancers in aggregate (FIG. 20A), and by each cancer type (FIG. 20B), in accordance with an embodiment of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for determining a tumor fraction in a subject. Such information can be used to determine the cancer status of the subject, including for instance classifying the tissue of origin of the subject. Sequence reads are obtained from a biological sample of a subject. The biological sample comprises cell-free nucleic acid. Thus, the sequence reads are of cell-free nucleic acid. The sequence reads are used to identify support for each variant in a variant set thereby determining an observed frequency of each variant. The observed variant frequencies are compared to corresponding reference frequencies for respective variants in a reference set. Each such reference frequency is a frequency of a respective variant in an aberrant tissue sample (e.g., a tumor) from the subject. In this way, the tumor fraction of the subject is determined. In some embodiments, the tumor fraction is used in conjunction with a classifier to classify a cancer condition of the subject.

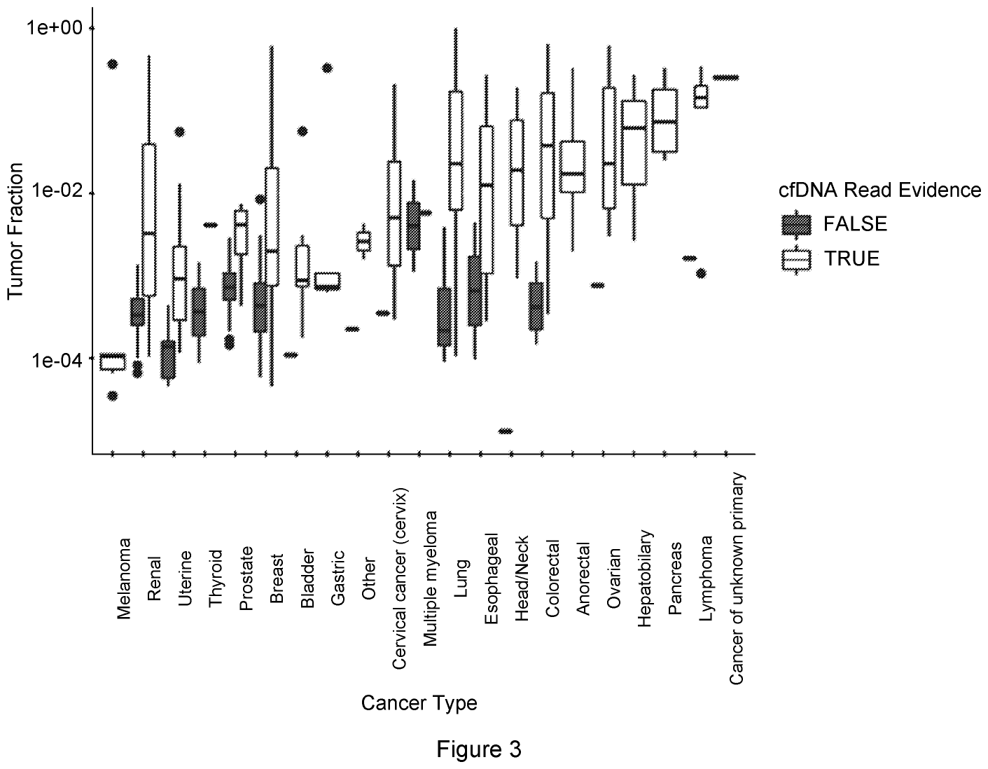

FIG. 3 provides one basis for the disclosed implementations. Typically, the observed frequency of the variants in the variant set, obtained from the cell-free nucleic acid of the biological sample, is less than the observed reference frequencies for such variants in the reference set. Without intending to be limited to any particular theory, it is presumed that the source of the cell-free nucleic acid that contains such variants is from decaying or broken up cancer cells in the aberrant tissue. As such, in some embodiments, the cell-free nucleic acid in the biological samples containing such variants in the disclosed variant sets of the present disclosure are presumed to represent ctDNA, or “circulating tumor DNA” (ctDNA) fraction of the cell free nucleic acids (cfDNA) used as the basis for determining observed frequencies of each variant. Therefore, it is expected that the observed frequency of the variants in the variant set, obtained from the cell-free nucleic acid of the biological sample, is less than the observed reference frequencies for such variants in the reference set. Data summarized in FIG. 3 support this contention, and moreover indicate that different cancer types have different ratios of observed frequency of the variants in the variant set for given subjects to the observed reference frequencies for such variants in a reference aberrant tissue in the same given subjects.

FIG. 3 provides a box plot in which, for each cancer type studied (regardless of stage of cancer) in a CCGA cohort, there are multiple individuals and an estimate of the ctDNA fraction for each individual is on the y-axis. FIG. 3 shows the summary of the distribution of ctDNA fraction observed for each respective cancer type for two classes of subjects for the respective cancer type: (i) those subjects having the respective cancer type in which there is no measured evidence of a variant (in the sequence reads from the cell-free biological samples) in their cfDNA (termed “FALSE” in FIG. 3) and (ii) those subjects having the respective cancer type in which there is measured evidence of a variant (in the sequence reads from the cell-free biological samples) in their cfDNA (termed “TRUE” in FIG. 3). For each respective cancer type, a first distribution of the measured ctDNA of the subjects in the TRUE category forms a first box (white boxes in FIG. 3) and a second distribution of the expected ctDNA of the subjects in the FALSE category forms a second box (grey filled boxes in FIG. 3), where the 25^{th} quantile and 75^{th} quantile define each such box, and the whiskers for each box show the extremes. The black line in each box is the median tumor fraction estimate for all of the individuals of a given cancer type of a given category. For instance, referring to renal cancer, there is a median ctDNA fraction for those subjects in the FALSE category and a different median ctDNA fraction for those subjects in the TRUE category.

FIG. 3 illustrates that there is a large dynamic range for the shedding rates (ctDNA fraction) of different cancers in the CCGA cohort studied. Details of the CCGA cohort are provided in Example 12 below. The observed large dynamic range can be used to inform a basis for establishing meaningful and informative thresholds, from observed frequencies of the variants in the reference set. That is, for example, given observed frequencies of variants in the aberrant tissue of a given subject, and optionally information regarding expected ctDNA fraction for subjects having a particular condition, a threshold for the given cancer subject is determined and evaluated against the observed frequency of the variants in a variant set for the given subject in order to classify the subject as having or not having the condition. For example, referring to FIG. 3, a threshold of 0.01 may be used to analyze whether a subject has renal cancer. In this example, an aberrant tissue, such as a tumor is obtained from a patient and used to determine a reference frequency for each respective variant in a first reference set. In fact, in some embodiments, the frequency of various possible variants is used to define the variants of the reference set. Next, cell free nucleic acid is obtained from a biological sample, other than the aberrant tissue, and the variant frequency of the same variants that are in the reference set are determined from sequence reads of the cell free nucleic acids in the biological sample, thereby forming the observed ctDNA frequency of each respective variant in the first variant set. A comparison of the ctDNA frequency to the reference frequencies to determine if the threshold condition of 0.01 is satisfied then provides a basis for determining whether or not the subject has renal cancer. For instance, if the comparison indicates that the ctDNA fraction is more than 0.01, this indicates that the subject does not have renal cancer. On the other hand, observation of a ctDNA fraction, formed from the observed frequency of each respective variant in the first variant set that is about 1e-03, is consistent with a finding of renal cancer. Moreover, in some embodiments, rather than indicating, on an absolute binary basis whether or not a subject has a particular condition using the disclosed systems and methods, a likelihood or probability that a subject has a particular condition is provided. In such embodiments, the comparison of the observed frequency of each respective variant in the first variant set to a corresponding reference frequency for the respective variant in a first reference set is used to determine how far apart the observed frequency of each respective variant in the first variant set to a corresponding reference frequency for the respective variant and, based on this distance or function of this distance, the probability or likelihood that a subject has a given condition.

In FIG. 3, the method used to compute the ctDNA fraction is a Bayesian method. For instance, consider the case where there is a tumor sequencing set of variants for a respective cancer type (reference set), and the matched cell-free DNA for a collection of subjects having the cancer type. If none of the tumor variants for the respective cancer type are matched to the cfDNA of any of the subject in the collection of subject, the collection of subjects can still be used to estimate what the ctDNA fraction would be for a respective cancer type in the absence of any supporting sequencing data thereby providing an upper bound on how much available signal there would be even though it was missed in the cell free nucleic assay. For computation of FIG. 3, on average there are 3000 sequence reads obtained from the cell free DNA from a biological sample of each subject. If none of the sequence reads support (contain) the variant in the 3000 sequence reads, while the subject is in the FALSE category of FIG. 3, this information can still be used to estimate what the potential underlying frequency (posterior probability) of that variant in the biological sample even though none of the sequence reads supported (contained) the variant. Such analysis forms the basis for the estimated ctDNA fraction provided for the subjects in the FALSE category for each cancer represented in FIG. 3. That is, the grey boxes in FIG. 3 represent the biological samples where no single variant associated with the cancer have been measured in the respective biological samples. These biological samples are used to independently estimate the potential underlying ctDNA fraction for the cancer. As FIG. 3 demonstrates, on median, such biological samples produce a reduced median ctDNA fraction relative to the median ctDNA fraction calculated using the population of samples for the same cancer in which variants associated with the cancer have been observed in the biological samples.

### Definitions

As disclosed herein, the term “biological sample” refers to any sample taken from a subject, which can reflect a biological state associated with the subject, and that includes cell free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject.

As disclosed herein, the terms “cell free nucleic acid,” “cell free DNA,” and “cfDNA” interchangeably refer to nucleic acid fragments that circulate in a subject's body (e.g., bloodstream) and originate from one or more healthy cells and/or from one or more cancer cells.

As disclosed herein, the term “circulating tumor DNA” or “ctDNA” refers to nucleic acid fragments that originate from aberrant tissue, such as the cells of a tumor or other types of cancer, which may be released into a subject's bloodstream as result of biological processes such as apoptosis or necrosis of dying cells or actively released by viable tumor cells.

As disclosed herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids are used interchangeably as circulating nucleic acids. Examples of the cell-free nucleic acids include but are not limited to RNA, mitochondrial DNA, or genomic DNA.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide that's not cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region only includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

As disclosed herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As disclosed herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of genomic sequence. In some embodiments, a method can include analysis of multiple mapped sequence reads to a plurality of genomic regions. Genomic regions can be approximately the same length or the genomic sections can be different lengths. In some embodiments, genomic regions are of about equal length. In some embodiments genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome or all or part of two or more chromosomes. In some embodiments, genomic regions may span one, two, or more entire chromosomes. In addition, the genomic regions may span joint or disjointed portions of multiple chromosomes.

As disclosed herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp.

As disclosed herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As disclosed herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As disclosed herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a women or a child).

### Exemplary System Embodiments

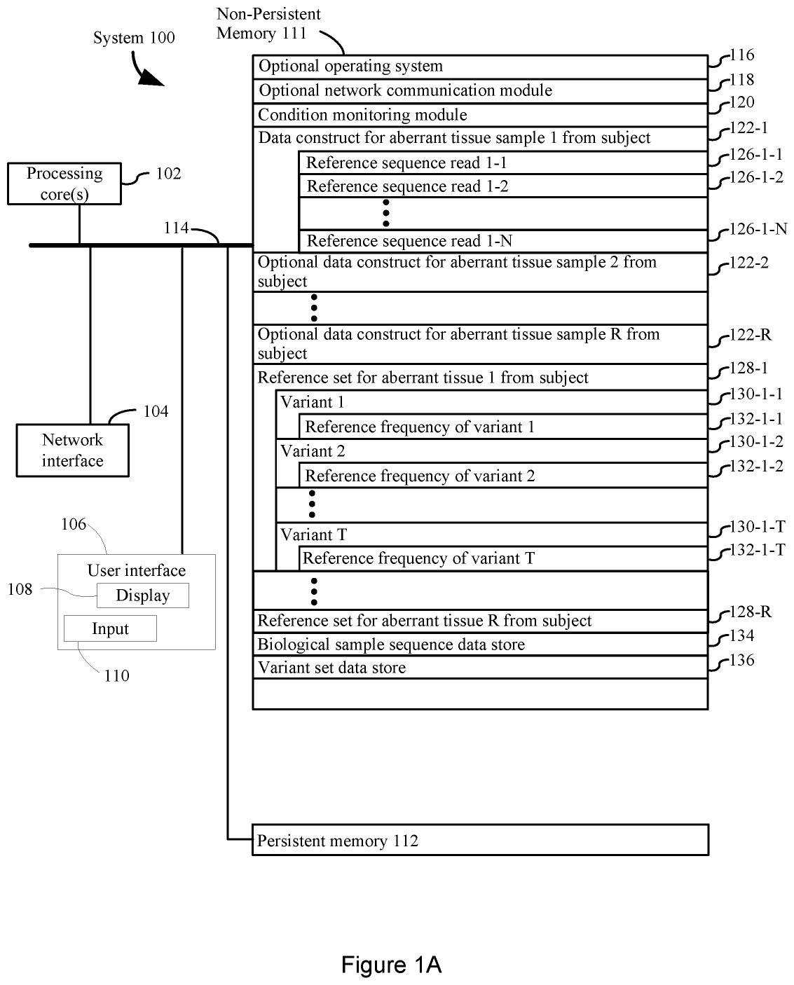

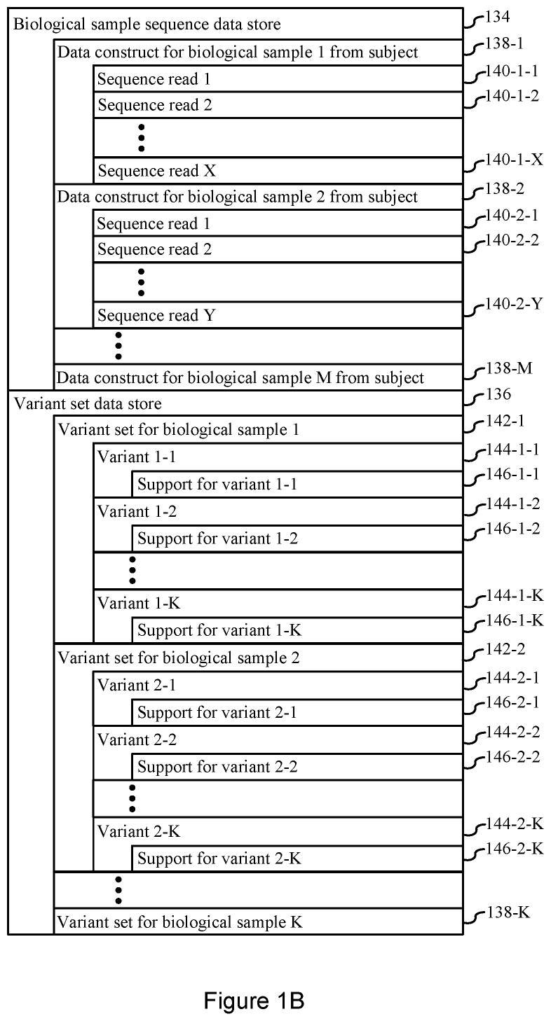

Now that an overview of some aspects of the present disclosure and some definitions used in the present disclosure have been provided, details of an exemplary system are now described in conjunction with FIG. 1. FIG. 1 is a block diagram illustrating a system 100 in accordance with some implementations. The device 100 in some implementations includes one or more processing units CPU(s) 102 (also referred to as processors), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices, or a communication network;
a condition monitoring module 120 for classifying a subject and/or evaluating a state of a condition in a subject and/or determining or monitoring a ctDNA tumor fraction of a subject;
one or more data constructs 122 for one or more aberrant dataset tissue samples from a subject, each such data construct 122 comprising a second plurality of sequence reads 126;
one or more reference sets 128, each respective reference set 128 for a corresponding data construct 122 for an aberrant tissue sample, and comprising an identification of each variant 130 in a set of variants and a reference frequency 132 of each such variant;
a biological sample sequence store 134 that comprises a respective data construct 138 for each corresponding biological sample from the subject, the corresponding biological sample comprising cell-free nucleic acid molecules, the respective data construct 138 comprising a first plurality of sequence reads 140 of such cell-free nucleic acid molecules; and
a variant set data store 136 comprising a variant set 142 for each corresponding biological sample, each such variant set 142 comprising a set of variants 144, each variant including a representation of the support for the first variant in the corresponding biological sample.

In some implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

Exemplary Method Embodiment—Based on aberrant tissue sequencing, discovering a condition

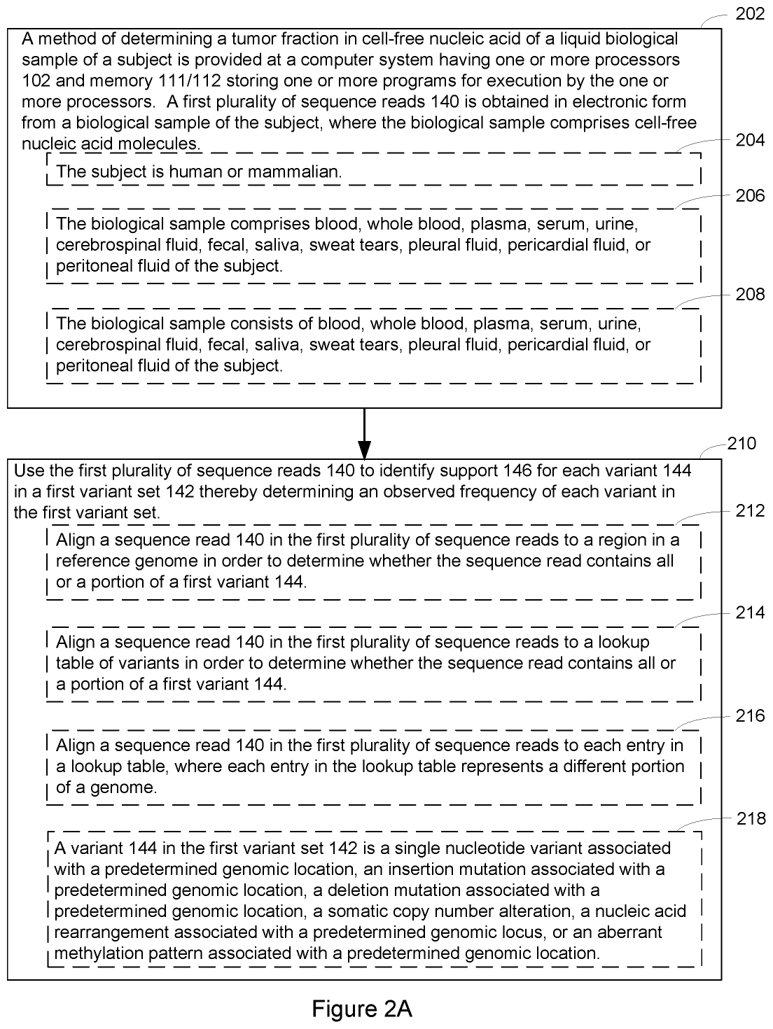

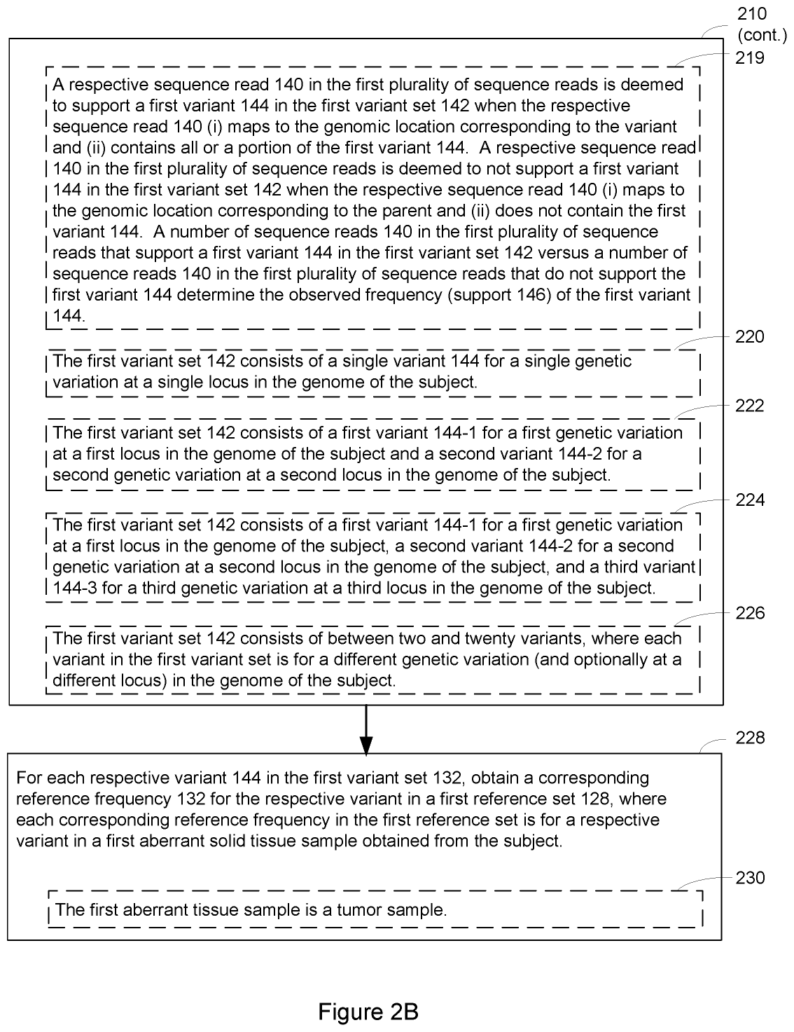

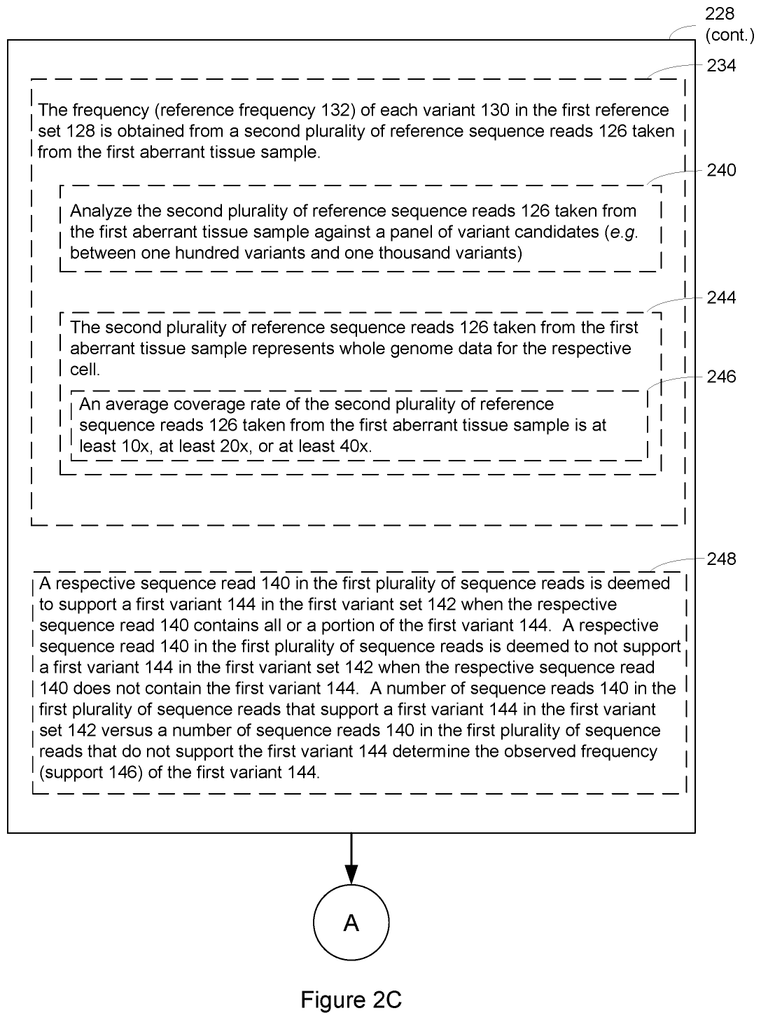

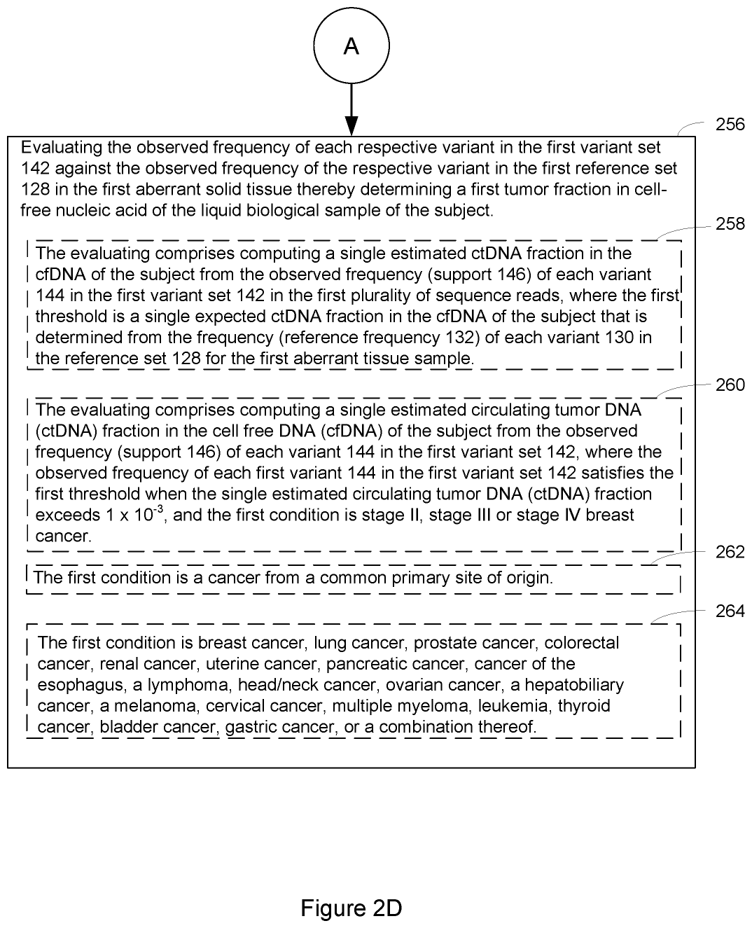

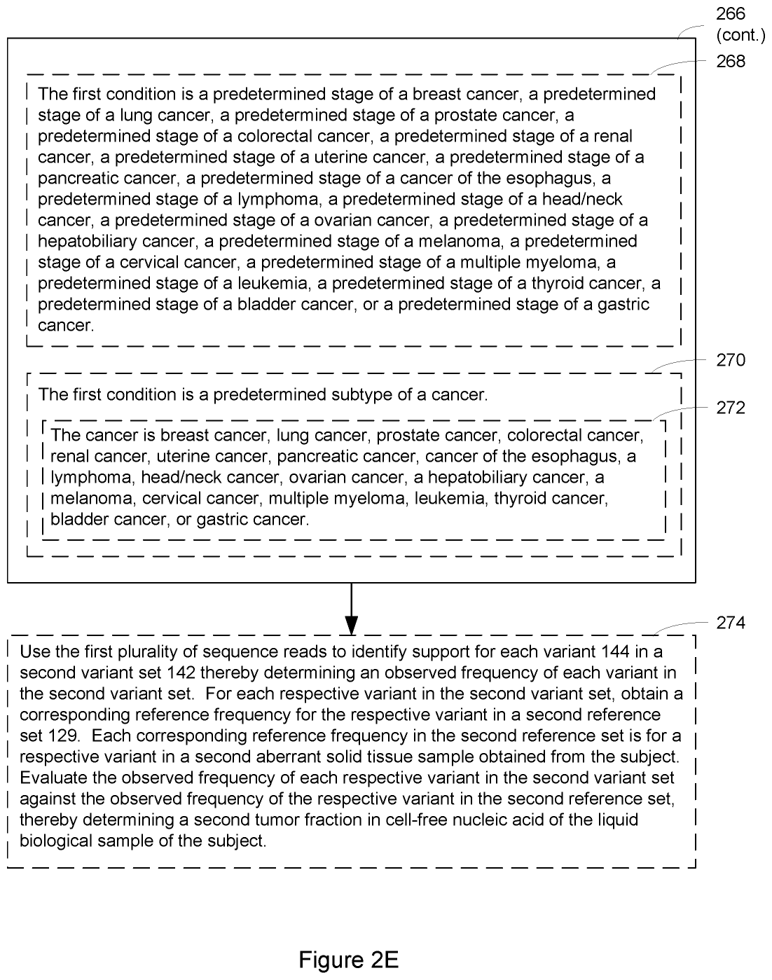

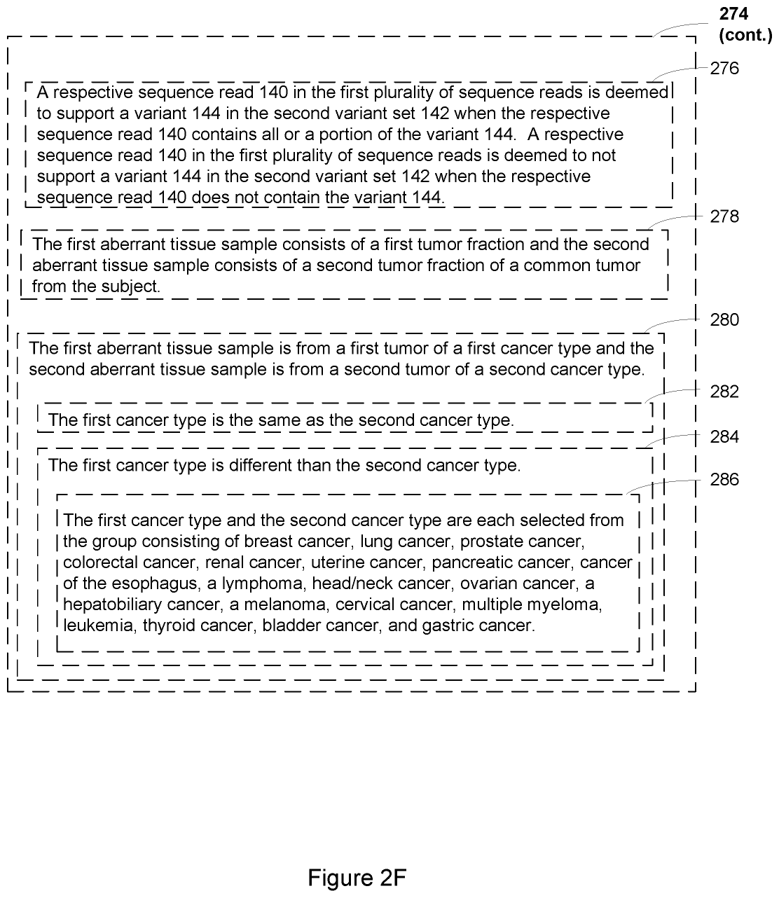

While a system in accordance with the present disclosure has been disclosed with reference to FIG. 1, a method in accordance with the present disclosure is now detailed with reference to FIG. 2.

Referring to blocks 202-208 of FIG. 2A, in some embodiments a method of determining a tumor fraction in cell-free nucleic acid of a liquid biological sample of a subject is performed at a computer system, such as system 100 of FIG. 1, which has one or more processors 102 and memory 111/112 storing one or more programs, such as condition monitoring module 120, for execution by the one or more processors. In some such embodiments, a first plurality of sequence reads 140 are obtained in electronic form from a biological sample of the subject, where the biological sample comprises cell-free nucleic acid molecules.

Referring to block 204, in some embodiments the subject is human or mammalian. In some embodiments, the subject is any living or non-living organism, including but not limited to a human (e.g., a male human, female human, fetus, pregnant female, child, or the like), a non-human animal, a plant, a bacterium, a fungus or a protist. In some embodiments, the subject is a mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

In some embodiments, the biological sample comprises blood, whole blood, plasma, serum, urine, cerebrospinal fluid, fecal, saliva, sweat, tears, pleural fluid, pericardial fluid, or peritoneal fluid of the subject (block 206). In such embodiments, the biological sample may include the blood, whole blood, plasma, serum, urine, cerebrospinal fluid, fecal, saliva, sweat, tears, pleural fluid, pericardial fluid, or peritoneal fluid of the subject as well as other components (e.g., solid tissues, etc.) of the subject.

In some embodiments, the biological sample consists of blood, whole blood, plasma, serum, urine, cerebrospinal fluid, fecal, saliva, sweat, tears, pleural fluid, pericardial fluid, or peritoneal fluid of the subject (block 208). In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject.

In some embodiments, the biological sample is processed to extract cell-free nucleic acids in preparation for sequencing analysis. By way of a non-limiting example, in some embodiments, cell-free nucleic acid is extracted from a blood sample collected from a subject in K2 EDTA tubes. Samples are processed within two hours of collection by double spinning of the blood first at ten minutes at 1000 g then plasma ten minutes at 2000 g. The plasma is then stored in 1 ml aliquots at −80° C. In this way, a suitable amount of plasma (e.g. 1-5 ml) is prepared from the biological sample for the purposes of cell-free nucleic acid extraction. In some such embodiments cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton, et al., 2017, “Phylogenetic ctDNA analysis depicts early stage lung cancer evolution,” Nature, 545(7655): 446-451, which is hereby incorporated by reference. Other equivalent methods can be used to prepare cell-free nucleic acid from biological methods for the purpose of sequencing, and all such methods are within the scope of the present disclosure.

In some embodiments, the cell-free nucleic acid that is obtained from a biological sample is in any form of nucleic acid defined in the present disclosure, or a combination thereof. For example, in some embodiments, the cell-free nucleic acid that is obtained from a biological sample is a mixture of RNA and DNA.

Any form of sequencing can be used to obtain the sequence reads 140 from the cell-free nucleic acid obtained from the biological sample including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads 140 from the cell-free nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain sequence reads 140 from the cell-free nucleic acid obtained from the biological sample. In some such embodiments, millions of cell-free nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a cell-free nucleic acid sample can include a signal or tag that facilitates detection. In some such embodiments, the acquisition of sequence reads 140 from the cell-free nucleic acid obtained from the biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, sequence reads 140 are obtained in the manner described in the example assay protocol disclosed in Example 10. In some embodiments, steps are taken to make sure that each such sequence read represents a unique nucleic acid fragment in the cell-free nucleic acid in the biological sample. Depending on the sequencing method used, each such unique nucleic acid fragment may be represented by a number of sequence reads. In typical instances, this redundancy in sequence reads to unique nucleic acid fragments in the cell-free nucleic acid is resolved using multiplex sequencing techniques such as barcoding so that the number of sequence reads for a given allele represents the number of unique nucleic acid fragments in the cell-free nucleic acid in the biological sample that map onto the different portion of the genome of the species represented by the respective allele, rather than the actual raw total number of sequence reads in the plurality of sequence reads mapping to the respective allele. See Kircher et al., 2012, Nucleic Acids Research 40, No. 1 e3, which is hereby incorporated by reference, for example disclosure on barcoding. In some embodiments, such mapping allows only perfect matches. In some embodiments, such mapping allows some mismatching. In some embodiments, a program such as Bowtie 2 is used to perform such mapping. See, for example, Langmead and Salzberg, 2012, Nat Methods 9, pp. 357-359, for example disclosure on such mapping.

In some embodiments, the first plurality of sequence reads obtained in block 202 from cell-free nucleic acid of a biological sample comprise more than ten sequence reads of the cell-free nucleic acid, more than one hundred sequence reads of the cell-free nucleic acid, more than five hundred sequence reads of the cell-free nucleic acid, more than one thousand sequence reads of the cell-free nucleic acid, more than two thousand sequence reads of the cell-free nucleic acid, between more than twenty five hundred sequence reads and five thousand sequence reads of the cell-free nucleic acid, or more than five thousand sequence reads of the cell-free nucleic acid. In some embodiments, each of these sequence reads is of a different portion of the cell-free nucleic acid. In some embodiments, one sequence read 140 in the first plurality of sequence reads is of all or a same portion of the cell-free nucleic acid as another sequence read in the first plurality of sequence reads.

Referring to blocks 210 to 216 of FIG. 2A, the first plurality of sequence reads 140 is used to identify support 146 for each variant 144 in a first variant set 142 thereby determining an observed frequency of each variant in the first variant set. In some embodiments, each variant 144 in the first variant set 142 is obtained from the first plurality of sequence reads after noise modelling, joint modelling with white blood cells (WBC), and/or edge variant artifact modelling as disclosed in United States Patent Application No. 16/201,912, entitled “Models for Targeted Sequencing,” filed Nov. 27, 2018, which is hereby incorporated by reference.

Referring to block 219 of FIG. 2B, in some embodiments, a respective sequence read 140 in the first plurality of sequence reads is deemed to support a first variant 144 in the first variant set 142 when the respective sequence read (i) encompasses or is within a genomic position associated with the first variant and (ii) contains all or a portion of the first variant. A respective sequence read in the first plurality of sequence reads is deemed to not support the first variant in the first variant set when the respective sequence read (i) encompasses or is within a genomic position associated with the first variant and (ii) does not contain all or a portion of the first variant. For instance, consider the case of a first variant that is associated with a particular genomic location. Those sequence reads that encompass or are within this particular genomic location are evaluated to determine whether they support the variant. In other words, those sequence reads that uniquely map onto this particular genomic location are evaluated to determine whether they support the variant. If a sequence read encompasses or is within a genomic position and encodes the variant, the sequence read is deemed to support the variant. For instance, in the case where the variant is a single nucleotide variation, those sequence reads that both (i) encompass the genomic location corresponding to this single nucleotide variation and (ii) have the single nucleotide variation is deemed to support the variation. In another example, in the case where the variant is an insertion that is longer than the average length of the sequence reads, those sequence reads that are within the genomic location corresponding to this variation (e.g. map into the locus of the genome where this insertion is to be bound) and (ii) have all or a portion of the insertion will be deemed to support the variation.

In some embodiments, the first plurality of sequence reads 140 is used to identify support 146 for each variant 144 in a first variant set by aligning each sequence read 140 in the first plurality of sequence reads to a region in a reference genome in order to determine whether the sequence read contains all or a portion of a first variant 144 (block 214). The alignment of a sequence read 140 to a region in a reference genome involves matching sequences from one or more sequence reads 140 to that of the reference genome based on complete or partial identity between the sequences. Alignments can be done manually or by a computer algorithm, examples including the Efficient Local Alignment of Nucleotide Data (ELAND) computer program distributed as part of the Illumina Genomics Analysis pipeline. The alignment of a sequence read to the reference genome can be a 100% sequence match. In some embodiments, an alignment is less than a 100% sequence match (e.g., non-perfect match, partial match, partial alignment). In some embodiments, an alignment comprises a mismatch. In some embodiments, an alignment comprises 1, 2, 3, 4 or 5 mismatches. In some embodiments, such mismatches are indicative of, and support, a variant 144 in a first variant set. For instance, in the case where a variant 144 is a single nucleotide variant at a given position in the genome, an alignment of a sequence read that contains the variant to the genome is expected to have a mismatch between the sequence read and the genome at the position in the genome associated with the single nucleotide variant. Two or more sequences can be aligned using either strand. In some embodiments a nucleic acid sequence is aligned with the reverse complement of another nucleic acid sequence.

In alternative embodiments, the first plurality of sequence reads 140 is used to identify support 146 for each variant 144 in a first variant set by aligning a sequence read 140 in the first plurality of sequence reads to a lookup table of variants in order to determine whether the sequence read contains all or a portion of a first variant 144 (block 214). Thus, in such instances, rather than using each sequence read 140 to find an alignment anywhere in the entire genome of a subject, each sequence read 140 is aligned to each of the sequences in a lookup table, where each such sequence in the lookup table represents a variant 144 in the first variant set 142. As an example, consider again the case where a variant 144 is a single nucleotide variant associated with a given position in the genome. In this case, the lookup table will include, for the variant, a portion of the sequence of the genome in the vicinity of the associated position of the genome. In some instances, the size of this portion may depend on the type of sequencing method used to generated the sequence reads 140. As a non-limiting example, the fifty bases flanking the 3′ side of the position in the genome associated with the single nucleotide variant and the fifty bases flanking the 5′ side of the position in the genome associated with the single nucleotide variant are used to represent the variant in the lookup table. In some embodiments, as discussed below with respect to block 218, in some instances the variant is some other kind of variant, such as an insertion mutation associated with a particular position in the genome. In such instances, the variant is represented in the lookup table by a portion of the genome that is sufficient to align with a sequence read that contains all or a significant portion of this insertion mutation.

In some embodiments, the first plurality of sequence reads 140 is used to identify support 146 for each variant 144 in the first variant set 142 using a variant calling process such as HaplotypeCaller. See, for example, McKenna et al., 2010, “The Genome Analysis Toolkit: a MapReduce framework for analyzing next-generation DNA sequencing data,” Genome Research 20: 1297-303; and Van der Auwera, 2013, “From FastQ Data to High-Confidence Variant Calls: The Genome Analysis Toolkit Best Practices Pipeline,” Current Protocols In Bioinformatics 43:11.10.1-11.10.33 each of which is hereby incorporated by reference.

In some embodiments, the first plurality of sequence reads 140 is used to identify support 146 for each variant in the first variant set 142 using VarScan. See, for example, Koboldt et al., 2012, “VarScan 2: Somatic mutation and copy number alteration discovery in cancer by exome sequencing,” Genome Research, PMID: 22300766; and Koboldt et al., 2009, “VarScan: variant detection in massively parallel sequencing of individual and pooled samples,” Bioinformatics 25 (17): 2283-5, each of which is hereby incorporated by reference.

In some embodiments, the first plurality of sequence reads 140 is used to identify support 146 for each variant in the first variant set 142 using Strelka. See, for example, Kim, et al., 2017, “Strelka2: Fast and accurate variant calling for clinical sequencing applications,” bioRxiv doi: 10.1101/192872, which is hereby incorporated by reference.

In some embodiments, the first plurality of sequence reads 140 is used to identify support 146 for each variant in the first variant set 142 using SomaticSniper. See, for example, Larson et al., 2012, “SomaticSniper: identification of somatic point mutations in whole genome sequencing data,” Bioinformatics 28(3), pp. 311-317, which is hereby incorporated by reference.

In some embodiments, the first plurality of sequence reads 140 is used to identify support 146 for each variant in the first variant set 142 in accordance with Example 11. In some embodiments, the sequence reads 140 are pre-processed to correct biases or errors using one or more methods such as normalization, correction of GC biases, and/or correction of biases due to PCR over-amplification.

In some embodiments, UMIs and endpoint positions of sequence reads collected in accordance with the present disclosure are used to define bags of likely PCR duplicates, which are collapsed (thereby obtaining a mean collapsed coverage) and stitched to high-accuracy fragment sequences. Accordingly, in such embodiments, “coverage” reported for a plurality of sequence reads is the mean collapsed coverage of such bags. In some embodiments, candidate variants are generated using a De Bruijn assembler, and are scored by a noise model trained on a cohort of non-smoking participants below 35 years of age without a diagnosis of cancer, used to measure technical variation from the sequencing assay. The noise model provides a calibrated quality score estimated on the support for each variant, allowing for filtering of candidate variants to a high-quality subset of variants unlikely to occur by purely technical variation. For instance, in the case of targeted sequencing such as the ART sequencing, the noise models and heuristic algorithms for identifying variants disclosed in U.S. patent application Ser. No. 16/201912 entitled “Models for Targeted Sequencing,” filed Nov. 27, 2018, are used in some embodiments of the present disclosure. In the case of whole genome sequencing, the noise models and heuristic algorithms for identifying variants disclosed in U.S. patent application Ser. No. 16/352,214 entitled “Identifying Copy Number Aberrations,” filed Mar. 13, 2019, are used in some embodiments of the present disclosure. Candidate variants were further filtered against DNA damage artifacts that clustered near the ends of reads and occurred in a subset of samples. Variants that are estimated to have phred score of 60 or higher and were unlikely to be technical artifacts are deemed to be variants in some embodiments. Variants that were estimated to have phred score 40 or higher, 45 or higher, 50 or higher, 55 or higher, 60 or higher, 65 or higher, or 70 or higher and are unlikely to be technical artifacts are deemed to be variants in some embodiments.

Use of epigenetic features such as methylation as variants. In some embodiments, the first plurality of sequence reads 140 is used to identify support 146 for each variant in the first variant set 142 by determining one or more methylation state vectors in accordance with Example 13 and as further disclosed in U.S. patent application Ser. No. 62/642,480, entitled “Methylation Fragment Anomaly Detection,” filed Mar. 13, 2018, which is hereby incorporated by reference. In such embodiments, five-cytosine methylation occurs at CpG contexts. One method for determining methylation status is through bisulfite conversion sequencing (BS-seq). Under BS-seq non-methylated cytosines are converted to uracil bases, which read out as thymidine in sequencing. Accordingly, in some embodiments, an epigenetic pattern such as the methylation state at one or more nucleotide positions is used as a basis for determining a variant allele for which ctDNA fraction is determined. In some embodiments, the methylation can include a methylation index of a CpG site, a methylation density of CpG sites in a region (e.g., that includes 2 or more, 3 or more, 4 or more 5 or more or 6 or more CpG sites), a distribution of CpG sites over a contiguous region, a pattern or level of methylation for each individual CpG site within a region that contains more than one CpG site, and/or non-CpG methylation. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine. In some embodiments, the cell free nucleic acid fragments are treated to convert unmethylated cytosines to uracils. In one embodiment, the method uses a bisulfite treatment of the DNA that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.) or by using the techniques disclosed in Schutsky et al., 2018, “Nondestructive, base-resolution sequencing of 5-hydroxymethylcytosine using a DNA deaminase,” Nature Biotechnology 36, 1083-1090 or Liu et al., 2019, “Bisulfite-free direct detection of 5-methylcytosine and 5-hydroxymethylcytosine at base resolution” Nature Biotechnology 37, pp. 424-429. From the converted cell free nucleic acid fragments, a sequencing library is prepared. Optionally, the sequencing library is enriched for cell free nucleic acid fragments, or genomic regions, that are informative for cell origin using a plurality of hybridization probes. The hybridization probes are short oligonucleotides that hybridize to particularly specified cell free nucleic acid fragments, or targeted regions, and enrich for those fragments or regions for subsequent sequencing and analysis. In some embodiments, hybridization probes are used to perform a targeted, high-depth analysis of a set of specified CpG sites that are informative for cell origin. Once prepared, the sequencing library or a portion thereof is sequenced to obtain a plurality of sequence reads. In alternative embodiments, whole genome bisulfite sequencing is performed as described for the CCGA study in Example 12 (WGBS; 34×).

Use of methylation sequencing data to ascertain variants. In some embodiments, whole-genome bisulfite sequencing (WGBS) or targeted bisulfite sequencing is used to procure the sequence reads 140. For example, in some embodiments, the WGBS, at a coverage rate of 34×, of the CCGA study described in Example 12 is used. In some embodiments, the coverage rate of such (WGBS) is 100× or less, 50× or less or between 30× and 200×. In typical embodiments, sequence read unique molecule indicators (UMIs) and endpoint positions are used to define likely PCR duplicates, which are collapsed into a bags in order to arrive at such coverage statistics. In some embodiments, a single sequence read from each bag is used in the disclosed analysis. In some embodiments, this single sequence read is a consensus sequence read. In some embodiments, this single sequence read is any sequence read in a bag. Thus, in this way, 100× refers to the number of unique fragments that cover each allele position, rather than the number of sequence reads that cover each allele position, since such sequence reads can include PCR duplicates. Such sequence reads, from the collapsed bags, can be used to detect sequencing variations (e.g., single nucleotide variants, insertions, deletions) or copy number variations. In some embodiments where sequence reads are used to identify single nucleotide variants, variants that are either C→T or T→C between non-cancer and cancer are not used because of the conversion of non-methylated cytosines to uracil bases, which read out as thymidine in sequencing; for example, by including a variant noise filter in a noise model for variant calling. In some embodiments, the noise model is modified to including one or more parameters to account for the strand origin of a sequence read (e.g., whether the read is from the forward or reverse strand of the original target molecule). Additional factors can be taken into consideration, including but not limited to trinucleotide context, position in the fragment of the variant and different kinds of other covariates. In some embodiments where sequence reads are used to identify single nucleotide variants, variants that are either C→T or T→C between non-cancer and cancer are in fact used provided that the bisulfite treatment of the DNA converts the unmethylated cytosines to uracils without converting the methylated cytosines. This can be done, for example, when a commercial kit such as the EZ DNA Methylation™—Gold, EZ DNA Methylation™—Direct or an EZ DNA Methylation—Lightning kit (available from Zymo Research Corp (Irvine, Calif.))) or the techniques disclosed in Schutsky et al., 2018, “Nondestructive, base-resolution sequencing of 5-hydroxymethylcytosine using a DNA deaminase,” Nature Biotechnology 36, 1083-1090 or Liu et al., 2019, “Bisulfite-free direct detection of 5-methylcytosine and 5-hydroxymethylcytosine at base resolution” Nature Biotechnology 37, pp. 424-429, is used for the bisulfite conversion. From the converted cell free nucleic acid fragments, a sequencing library is prepared. Optionally, the sequencing library is enriched for cell free nucleic acid fragments, or genomic regions, that are informative for cell origin using a plurality of hybridization probes. The hybridization probes are short oligonucleotides that hybridize to particularly specified cell free nucleic acid fragments, or targeted regions, and enrich for those fragments or regions for subsequent sequencing and analysis. In some embodiments, hybridization probes are used to perform a targeted, high-depth analysis of a set of specified CpG sites that are informative for cell origin. Once prepared, the sequencing library or a portion thereof is sequenced to obtain a plurality of sequence reads. In alternative embodiments, whole genome bisulfite sequencing is performed as described for the CCGA study in Example 12 (WGBS; 34×).

Whole genome plasma assay. In some embodiments, the subject is human and the first plurality of sequence reads 140 taken from the biological sample are part of a whole genome plasma assay.

In some such embodiments, the whole genome plasma assay is conducted using cfDNA extracted from two tubes of plasma (up to a combined volume of 10 ml) of a modified QIAamp Circulating Nucleic Acid kit (Qiagen; Germantown, Md.). Genomic DNA (gDNA) from buffy coat was extracted using Qiagen DNEasy Blood and Tissue kit, is quantified using NanoDrop (Thermo Scientific; Waltham, Mass.). Extracted gDNA is fragmented using Covaris E220 ultrasonicator (Woburn, Mass.), and was size-selected using Agencourt AMPure XP magnetic beads (Beckman Coulter; Beverly, Mass.). Plasma cfDNA (up to 75 ng) and buffy coat gDNA (75 ng) were used for next generation sequencing (NGS) library construction. The adapter included a set of 218 unique molecular identifier (UMI) sequences to reduce assay and sequencing errors. A fraction of amplified libraries (4 μL of 25 μL) are diluted and quantified using AccuClear Ultra High-Sensitivity dsDNA Quantitation kit (Biotium; Fremont, Calif.). The remainder is used in a targeted sequencing protocol (see below). Three or four diluted libraries were normalized, pooled, clustered on a flowcell, and sequenced on an Illumina HiSeq X (30×).

The sequence reads 140 are compared to the entire human genome in order to identify variants. In some embodiments, the first plurality of sequence reads 140 taken from the biological sample have at least 30× coverage for a targeted panel of genes, at least 40× coverage for a targeted panel of genes, at least 50× coverage for a targeted panel of genes, at least 60× coverage for a targeted panel of genes, or at least 70× coverage for a targeted panel of genes. In some such embodiments, the targeted panel of genes is between 450 and 500 fifty genes. In some embodiments, the targeted panel of genes is within the range of 500±5 genes, within the range of 500±10 genes, or within the range 500±25 genes. In some embodiments, the whole genome assay plasma looks for somatic copy number alterations (SCNAs) or fragmented features in the genome.

Targeted plasma assay. In some embodiments, the subject is a human and the first plurality of sequence reads 140 taken from the biological sample are part of a targeted plasma assay.

In some such embodiments, the amplified libraries (see Whole genome plasma assay section above) are used for target enrichment with a panel targeting 507 cancer-related genes as part of the ART assay disclosed in Example 12. Up to 3.5 μg of each library underwent hybridization-based capture. The enriched libraries are quantified using AccuClear Ultra High-Sensitivity dsDNA Quantitation kit. Three or four enriched libraries are normalized, pooled, clustered on a flowcell, and sequenced on Illumina HiSeq X (150-bp paired-end sequencing, 60,000×).

The sequence reads 140 acquired in this manner are compared to a targeted panel of genes of the targeted plasma assay in order to identify variants. In some such embodiments, the targeted panel of genes is between 450 and 500 fifty genes. In some embodiments, the targeted panel of genes is within the range of 500±5 genes, within the range of 500±10 genes, or within the range 500±25 genes. In some embodiments, the first plurality of sequence reads 140 taken from the biological sample have at least 50,000× coverage for this targeted panel of genes, at least 55,000× coverage for this targeted panel of genes, at least 60,000× coverage for this targeted panel of genes, or at least 70,000× coverage for this targeted panel of genes. In some such embodiments, the targeted plasma assay looks for single nucleotide variants in the targeted panel of genes, insertions in the targeted panel of genes, deletions in the targeted panel of genes, somatic copy number alterations (SCNAs) in the targeted panel of genes, aberrant methylation patterns, or re-arrangements affecting the targeted panel of genes.

Targeted white blood cell assay. In some embodiments, the subject is a human and the first plurality of sequence reads 140 taken from the biological sample are part of a targeted white blood cell assay. That is, the biological sample is white blood cells from the subject and the sequence reads 140 are compared to a targeted panel of genes of the targeted white blood cell assay in order to identify variants. In some such embodiments, the targeted panel of genes is between 450 and 500 fifty genes. In some embodiments, the targeted panel of genes is within the range of 500±5 genes, within the range of 500+10 genes, or within the range 500+25 genes. In some embodiments, the first plurality of sequence reads 140 taken from the biological sample have at least 50,000× coverage for this targeted panel of genes, at least 55,000× coverage for this targeted panel of genes, at least 60,000× coverage for this targeted panel of genes, or at least 70,000× coverage for this targeted panel of genes. In some such embodiments, the targeted white blood cell assay looks for single nucleotide variants in the targeted panel of genes, insertions in the targeted panel of genes, deletions in the targeted panel of genes, or somatic copy number alterations (SCNAs) in the targeted panel of genes.

Whole genome white blood cell assay. In some embodiments, the subject is human and the first plurality of sequence reads 140 taken from the biological sample are part of a whole genome white blood cell assay. That is, the biological sample is white blood cells from the subject and the sequence reads 140 are compared to the entire human genome in order to identify variants. In some embodiments, the first plurality of sequence reads 140 taken from the biological sample have at least 30× coverage for a targeted panel of genes, at least 40× coverage for a targeted panel of genes, at least 50× coverage for a targeted panel of genes, at least 60× coverage for a targeted panel of genes, or at least 70× coverage for a targeted panel of genes. In some such embodiments, the targeted panel of genes is between 450 and 500 fifty genes. In some embodiments, the targeted panel of genes is within the range of 500±5 genes, within the range of 500±10 genes, or within the range 500±25 genes. In some embodiments, the whole genome white blood cell assay looks for somatic copy number alterations (SCNAs) or fragmented features in the genome.

Whole genome bisulfite sequencing assay. In some embodiments, the subject is human and the first plurality of sequence reads 140 are obtained through bisulfite sequencing and are evaluated for variants on a genome wide basis. In some embodiments, the whole genome bisulfite sequencing assay looks for variants in methylation patterns in the genome. See, for example, Example 13. See also, U.S. patent application Ser. No. 62/642,480, entitled “Methylation Fragment Anomaly Detection,” filed Mar. 13, 2018, which is hereby incorporated by reference.

In some embodiments, referring to block 216 of FIG. 2A, the first plurality of sequence reads 140 is used to identify support 146 for each variant 144 in a first variant set by aligning each sequence read 140 in the first plurality of sequence reads to each entry in a lookup table, where each entry in the lookup table represents a different portion of a genome (e.g., a reference genome). Such an embodiment is used in some instances to populate the lookup table with hotspots in the genome. Thus, rather than aligning each sequence read by searching for an alignment throughout the entire genome, each sequence read is aligned to only those portions of the genome, for instance genes within the genome, that have been associated with conditions of interest. For instance, consider the case where mutation of a particular gene has been associated with a clinical condition. In this case, in accordance with the embodiment of block 216, the genomic sequence of the gene can be included as an entry in the lookup table and sequence reads 140 can be aligned to this entry in order to identify support for a variant to the gene. In a variation of this case, each known mutation of the gene can be listed as a separate entry in the lookup table and each sequence read 140 in the first plurality of sequence reads can be aligned to each of these separate entries in order to determine if there is a match between the sequence read and one of the mutations of the genes, thereby identifying support for a variant 144 in the variant set.

In still another example, only those variants (and enough of the genomic sequence in the vicinity of such variants) found in an aberrant tissue (e.g., tumor) of a given subject are included in the lookup table. In this way, the process of identifying sequence reads 126 that match one of the variants in the tumor and thereby identify support for the variant is greatly speeded up over embodiments where the sequence reads are aligned to the entirety of a reference genome in order to identify support such variants. For instance, consider the case where sequencing of a given tumor in a subject identifies three variants. In this case, the three variants are provided as separate entries in the lookup table and each of the sequence reads 140 in the first plurality of sequence reads are independently aligned to each of the entries in the lookup table to determine whether they align with one of the variants and thereby support the variant.

In some embodiments, the lookup table consists of a single entry, where the single entry is a variant that has been identified in an aberrant tissue of a subject. In some embodiments, the lookup table consists of two entries, where each entry represents a variant that has been identified in an aberrant tissue of a subject. In some embodiments, the lookup table consists of three entries, where each entry represents a variant that has been identified in an aberrant tissue of a subject. In some embodiments, the lookup table consists of between three and ten entries, where each entry represents a variant that has been identified in an aberrant tissue of a subject.

In some embodiments, the lookup table comprises between two and one thousand entries where each entry represents a different gene in the human genome.

Referring to block 218 of FIG. 2A, in some embodiments a variant 144 in the first variant set 142 is a single nucleotide variant associated with a predetermined genomic location, an insertion mutation associated with predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location. By way of example, in some embodiments a variant 144 is a somatic mutation of a particular gene in the genome and thus is associated with the genomic location of the particular gene in the genome.

In some embodiments, the variant set 142 includes more than one type of variant. For example, in some embodiments, the variant set 142 includes a single nucleotide variant associated with one genomic location and a deletion mutation associated with another genomic location in a genome.

In some embodiments, a variant 144 is any form of somatic mutation.

In some embodiments, each of the variants 144 in the first variant set is also found in the reference set 128. In some such embodiments, there is a one-to-one correspondence between variants 144 in the variant set 142 and variants 130 in the reference set 128. In such embodiments, the variant set 142 includes the identified support 146 for the variants in the biological sample (e.g., blood) of the subject whereas the reference set 128 includes the reference frequency 132 of such variants in the aberrant tissue (e.g., tumor) of the subject.

In some embodiments, the first variant set 142 consists of a single variant 144 for a single genetic variation for a single locus in the genome of the subject (block 220). For instance, consider the case where a particular single nucleotide variant is found in a particular gene in a percentage of the sequence reads from the aberrant tissue (e.g., tumor) of a subject that map onto this particular gene. In this instance, the variant set 142 will also include the particular single nucleotide variant and any support 146 identified for this particular single nucleotide variant in the particular gene that is found in the first plurality of sequence reads obtained from the biological sample (e.g., blood) of the subject.

In some embodiments, the first variant set 142 consists of a first variant 144-1 for a first genetic variation at a first locus in the genome of the subject and a second variant 144-2 for a second genetic variation at a second locus in the genome of the subject (block 222). For instance, consider the case where a first variant is found in a first gene in some appreciable percentage (e.g., more than one percent, more than two percent, more than five percent) or number of the sequence reads of an aberrant tissue (e.g., tumor) of a subject that map onto this first gene and a second variant is found in a second gene in some appreciable percentage or number of the sequence reads of the aberrant tissue that map onto the second gene. In this instance, the variant set 142 will include the first variant and any support 146 identified for the first variant that is found in the first plurality of sequence reads obtained from the biological sample (e.g., blood) of the subject. The variant set 142 will also include the second variant and any support 146 identified for the second variant in the second gene that is found in the first plurality of sequence reads obtained from the biological sample.

In some embodiments, a variant is included in the reference set when at least one sequence read from the aberrant tissue supports the variant. In such embodiments, a sequence read from the aberrant tissue supports a variant when the sequence read (i) maps onto a genomic location associated with the variant and (ii) includes the variant. In some embodiments, a variant is included in the reference set when at least two sequence reads from the aberrant tissue support the variant. In some embodiments, a variant is included in the reference set when at least two sequence reads, at least five sequence reads, at least ten sequence reads, at least one hundred sequence reads, at least 200 sequence reads, or at least 1000 sequence reads from the aberrant tissue support the variant.

In some embodiments, the first variant set 142 consists of a first variant 144-1 for a first genetic variation at a first locus in the genome of the subject, a second variant 144-2 for a second genetic variation at a second locus in the genome of the subject, and a third variant 144-3 for a third genetic variation at a third locus in the genome of the subject (block 224). For instance, consider the case where a first variant is found in a first gene in some appreciable percentage or number of the sequence reads of an aberrant tissue (e.g., tumor) of a subject that include the first gene, a second variant is found in a second gene in some appreciable percentage or number of the sequence reads of the aberrant tissue that include the second gene, and a third variant is found in a third gene in some appreciable percentage or number of the sequence reads of the aberrant tissue that include the third gene. In this instance, the variant set 142 will include the first variant and any support 146 identified for the first variant that is found in the first plurality of sequence reads obtained from the biological sample (e.g., blood) of the subject. The variant set 142 will also include the second variant and any support 146 identified for the second variant in the second gene that is found in the first plurality of sequence reads obtained from the biological sample. The variant set 142 will also include the third variant and any support 146 identified for the third variant in the third gene that is found in the first plurality of sequence reads obtained from the biological sample.

In some embodiments, the first variant set 142 consists of between two and twenty variants, where each variant in the first variant set is for (represents) a different genetic variation at a different locus in the genome of the subject (block 226). In some embodiments, the first variant set 142 consists of between two and twenty variants, where each variant in the first variant set is for (represents) a different genetic variation in the genome of the subject (block 226). In some embodiments, each respective variant in the first variant set is also found in an appreciable percentage (e.g., more than one percent, more than two percent, more than five percent) or number of the sequence reads of an aberrant tissue (e.g., tumor) of a subject that map to the genomic location of the respective variant. In some embodiments, the first variant set 142 consists of between one and ten variants, where each variant in the first variant set is for (represents) a different genetic variation (and optionally at a different locus) in the genome of the subject. In some embodiments, the first variant set 142 consists of between one and one hundred variants, where each variant in the first variant set is for (represents) a different genetic variation (and optionally at a different locus) in the genome of the subject. In some embodiments, the first variant set 142 consists of between two and one hundred variants, where each variant in the first variant set is for (represents) a different genetic variation (and optionally at a different locus) in the genome of the subject. In some embodiments, the first variant set 142 consists of between one and one thousand variants, where each variant in the first variant set is for (represents) a different genetic variation (and optionally at a different locus) in the genome of the subject.

In some embodiments, a first variant and a second variant in the variant set are associated with the same locus in the genome of a subject. For instance, the first and second variant may represent two different aberrant alleles of the same gene.

For each respective variant in the first variant set, obtain a corresponding reference frequency for the respective variant in a first reference set, where each corresponding reference frequency in the first reference set is for a respective variant in a first aberrant solid tissue sample obtained from the subject. Referring to block 228 of FIG. 2B, in the disclosed methods, the observed frequency (e.g., support 146) of each respective variant 144 in the first variant set 142 is compared to a corresponding reference frequency 132 for the respective variant in a first reference set 128. Each corresponding reference frequency 132 in the first reference set 128 is a frequency of a respective variant 130 in a first aberrant tissue sample obtained from the subject.

Referring to block 230 of FIG. 2B, in some embodiments, the first aberrant tissue sample is a tumor sample, or a fraction thereof. In some embodiments, the first aberrant tissue sample an adrenocortical carcinoma, a childhood adrenocortical carcinoma, a tumor of an AIDS-related cancer, kaposi sarcoma, a tumor associated with anal cancer, a tumor associated with an appendix cancer, an astrocytoma, a childhood (brain cancer) tumor, an atypical teratoid/rhabdoid tumor, a central nervous system (brain cancer) tumor, a basal cell carcinoma of the skin, a tumor associated with bile duct cancer, a bladder cancer tumor, a childhood bladder cancer tumor, a bone cancer (e.g., ewing sarcoma and osteosarcoma and malignant fibrous histiocytoma) tissue, a brain tumor, breast cancer tissue, childhood breast cancer tissue, a childhood bronchial tumor, burkitt lymphoma tissue, a carcinoid tumor (gastrointestinal), a childhood carcinoid tumor, a carcinoma of unknown primary, a childhood carcinoma of unknown primary, a childhood cardiac (heart) tumor, a central nervous system (e.g., brain cancer such as childhood atypical teratoid/rhabdoid) tumor, a childhood embryonal tumor, a childhood germ cell tumor, cervical cancer tissue, childhood cervical cancer tissue, cholangiocarcinoma tissue, childhood chordoma tissue, a chronic myeloproliferative neoplasm, a colorectal cancer tumor, a childhood colorectal cancer tumor, childhood craniopharyngioma tissue, a ductal carcinoma in situ (DCIS), a childhood embryonal tumor, endometrial cancer (uterine cancer) tissue, childhood ependymoma tissue, esophageal cancer tissue, childhood esophageal cancer tissue, esthesioneuroblastoma (head and neck cancer) tissue, a childhood extracranial germ cell tumor, an extragonadal germ cell tumor, eye cancer tissue, an intraocular melanoma, a retinoblastoma, fallopian tube cancer tissue, gallbladder cancer tissue, gastric (stomach) cancer tissue, childhood gastric (stomach) cancer tissue, a gastrointestinal carcinoid tumor, a gastrointestinal stromal tumor (GIST), a childhood gastrointestinal stromal tumor, a germ cell tumor (e.g., a childhood central nervous system germ cell tumor, a childhood extracranial germ cell tumor, an extragonadal germ cell tumor, an ovarian germ cell tumor, or testicular cancer tissue), head and neck cancer tissue, a childhood heart tumor, hepatocellular cancer (HCC) tissue, an islet cell tumor (pancreatic neuroendocrine tumors), kidney or renal cell cancer (RCC) tissue, laryngeal cancer tissue, leukemia, liver cancer tissue, lung cancer (non-small cell and small cell) tissue, childhood lung cancer tissue, male breast cancer tissue, a malignant fibrous histiocytoma of bone and osteosarcoma, a melanoma, a childhood melanoma, an intraocular melanoma, a childhood intraocular melanoma, a merkel cell carcinoma, a malignant mesothelioma, a childhood mesothelioma, metastatic cancer tissue, metastatic squamous neck cancer with occult primary tissue, a midline tract carcinoma with NUT gene changes, mouth cancer (head and neck cancer) tissue, multiple endocrine neoplasia syndrome tissue, a multiple myeloma/plasma cell neoplasm, myelodysplastic syndrome tissue, a myelodysplastic/myeloproliferative neoplasm, a chronic myeloproliferative neoplasm, nasal cavity and paranasal sinus cancer tissue, nasopharyngeal cancer (NPC) tissue, neuroblastoma tissue, non-small cell lung cancer tissue, oral cancer tissue, lip and oral cavity cancer and oropharyngeal cancer tissue, osteosarcoma and malignant fibrous histiocytoma of bone tissue, ovarian cancer tissue, childhood ovarian cancer tissue, pancreatic cancer tissue, childhood pancreatic cancer tissue, papillomatosis (childhood laryngeal) tissue, paraganglioma tissue, childhood paraganglioma tissue, paranasal sinus and nasal cavity cancer tissue, parathyroid cancer tissue, penile cancer tissue, pharyngeal cancer tissue, pheochromocytoma tissue, childhood pheochromocytoma tissue, a pituitary tumor, a plasma cell neoplasm/multiple myeloma, a pleuropulmonary blastoma, a primary central nervous system (CNS) lymphoma, primary peritoneal cancer tissue, prostate cancer tissue, rectal cancer tissue, a retinoblastoma, a childhood rhabdomyosarcoma, salivary gland cancer tissue, a sarcoma (e.g., a childhood vascular tumor, osteosarcoma, uterine sarcoma, etc.), Sézary syndrome (lymphoma) tissue, skin cancer tissue, childhood skin cancer tissue, small cell lung cancer tissue, small intestine cancer tissue, a squamous cell carcinoma of the skin, a squamous neck cancer with occult primary, a cutaneous t-cell lymphoma, testicular cancer tissue, childhood testicular cancer tissue, throat cancer (e.g., nasopharyngeal cancer, oropharyngeal cancer, hypopharyngeal cancer) tissue, a thymoma or thymic carcinoma, thyroid cancer tissue, transitional cell cancer of the renal pelvis and ureter tissue, unknown primary carcinoma tissue, ureter or renal pelvis tissue, transitional cell cancer (kidney (renal cell) cancer tissue, urethral cancer tissue, endometrial uterine cancer tissue, uterine sarcoma tissue, vaginal cancer tissue, childhood vaginal cancer tissue, a vascular tumor, vulvar cancer tissue, a Wilms tumor or other childhood kidney tumor.

In some embodiments, the sequence reads from the first aberrant tissue sample are formalin-fixed paraffin-embedded (FFPE) tumor tissue sections that are scraped and sent to the Genome Services Lab at HudsonAlpha Institute for Biotechnology (Huntsville, Alabama), where DNA is extracted from the scrapings and converted into NGS libraries for whole-genome sequencing on an Illumina HiSeq X (30×). For each tissue scraping, one tube of corresponding buffy coat is shipped to HudsonAlpha for extraction, library preparation, and whole-genome sequencing on Illumina HiSeq X (60×). Sequencing data is then analyzed in accordance with the present disclosure.

Referring to blocks 234-240, in some embodiments the frequency (reference frequency 132) of each variant 130 in the first reference set 128 is obtained from a second plurality of sequence reads (plurality of reference sequence reads) 126 taken from the first aberrant tissue sample (block 234). In some embodiments, the frequency of a respective variant 130 is a measure of the proportion of cells in the first aberrant tissue of the subject in which the variant resides. See, for example, Lu et al., 2015 “Allele frequency of somatic mutations in individuals reveals signatures of cancer-related genes,” Acta Biochim Biophys Sin. 47(8), 657-680, which is hereby incorporated by reference, for disclosure on determining frequency of somatic variants in aberrant tissue in accordance with some embodiments.

In some embodiments, the frequency of a respective variant 130 is determined by first identifying the sequence reads that could potentially have the respective variant 130. For instance, if the respective variant is a single nucleotide variant, the sequence reads from the first aberrant tissue that map to the genomic location corresponding to this respective variant are identified. Then, the proportion of these identified sequence reads that include the variant represent the frequency of the respective variant. Thus, if there are 200 sequence reads from the aberrant tissue that map to the genomic location that is associated with the variant, and 50 of these sequence reads include the allele for the variant whereas the remaining 150 sequence reads have a wild type allele rather than the allele for the variant, the frequency for the respective variant 130 is 25 percent. In some such embodiments, steps are taken to make sure that each such sequence read represents a unique nucleic acid fragment in the aberrant tissue. Depending on the sequencing method used, each such unique nucleic acid fragment may be represented by a number of sequence reads. In typical instances, this redundancy in sequence reads to unique nucleic acid fragments in the aberrant solid tissue sample is resolved using multiplex sequencing techniques such as barcoding so that the number of sequence reads for a given allele represents the number of unique nucleic acid fragments in the aberrant solid tissue sample that map onto the different portion of the genome of the species represented by the respective allele, rather than the actual raw total number of sequence reads in the plurality of sequence reads mapping to the respective allele. See Kircher et al., 2012, Nucleic Acids Research 40, No. 1 e3, which is hereby incorporated by reference, for example disclosure on barcoding.

In some embodiments, more than 1000, 2000, 3000, 4000, 5000, 10,000, 20,000, 100,000 or one million reference sequence reads 126 are taken from the aberrant tissue. In some embodiments, the reference sequence reads 126 taken from the aberrant tissue provide a coverage rate of 1× or greater, 2× or greater, 5× or greater, 10× or greater, or 50× or greater for at least two percent, at least five percent, at least ten percent, at least twenty percent, at least thirty percent, at least forty percent, at least fifty percent, at least sixty percent, at least seventy percent, at least eighty percent, at least ninety percent, at least ninety-eight percent, or at least ninety-nine percent of the genome of the subject. In some embodiments, the reference sequence reads 126 taken from the aberrant tissue provide a coverage rate of 1× or greater, 2× or greater, 5× or greater, 10× or greater, or 50× or greater for at least three genes, at least five genes, at least ten genes, at least twenty genes, at least thirty genes, at least forty genes, at least fifty genes, at least sixty genes, at least seventy genes, at least eighty genes, at least ninety genes, at least 200 genes, at least 300 genes, at least 400 genes, at least 500 genes or at least 1000 genes of the genome of the subject.

In some embodiments, the plurality of reference sequence reads 126 taken from the first aberrant tissue are analyzed against (aligned against) a panel of variant candidates. For instance, in some embodiments, the panel of variant candidates includes sequences for variant candidates of at least three genes, at least five genes, at least ten genes, at least twenty genes, at least thirty genes, at least forty genes, at least fifty genes, at least sixty genes, at least seventy genes, at least eighty genes, at least ninety genes, at least 200 genes, at least 300 genes, at least 400 genes, at least 500 genes or at least 1000 genes of the subject. To perform such an analysis, alignment of a particular reference sequence read 126 to the sequence of a variant candidate in the panel of variant candidates involves matching the sequence of the reference sequence read 126 to that of the sequence of the variant candidate to see if there is complete or partial identity between the sequences. Such alignments (analysis) can be done manually or by a computer algorithm, examples including the Efficient Local Alignment of Nucleotide Data (ELAND) computer program distributed as part of the Illumina Genomics Analysis pipeline. In some embodiments, a reference sequence read 126 and the sequence of the variant candidate in the panel of variant candidates are deemed to match when 100% of the reference sequence read 126 matches a corresponding portion of the sequence of the variant candidate. In some embodiments, a reference sequence read 126 and the sequence of the variant candidate in the panel of variant candidates are deemed to match when 100% of the sequence of the variant candidate 126 matches a corresponding portion of the sequence of the reference sequence read 126. In some embodiments, an alignment is less than a 100% sequence match (e.g., non-perfect match, partial match, partial alignment). In some embodiments, an alignment comprises a mismatch. In some embodiments, an alignment comprises 1, 2, 3, 4 or 5 mismatches. Two or more sequences can be aligned using either strand. In some embodiments, a nucleic acid sequence is aligned with the reverse complement of another nucleic acid sequence.

Referring to blocks 244-246 of FIG. 2C, in some embodiments, the plurality of reference sequence reads 126 taken from the first aberrant tissue sample represents the whole genome data for the respective cell. In some such embodiments, an average coverage rate of the plurality of reference sequence reads 126 taken from the first aberrant tissue sample is at least 1×, 2×, 3×, 4×, 5×, 6×, 7×, 8×, 9×, 10×, at least 20×, at least 30×, or at least 40× across the genome of the subject. In some embodiments the average coverage rate of the second plurality of sequence reads across the first reference set 128 is at least 10×, at least 100×, or at least 2000×.

Referring to block 248 of FIG. 2C, in some embodiments a respective sequence read 126 in the second plurality of sequence reads is deemed to support a first variant 130 in the reference set 128 when the respective sequence read (i) maps to a portion of the genome associated with the first variant and (ii) the respective sequence read 126 contains all or a portion of the first variant 130. A respective sequence read 126 in the second plurality of sequence reads is deemed to not support a first variant 130 in the reference set 128 when the respective sequence read 126 (i) maps to a portion of the genome associated with the first variant 130 (genomic location corresponding to the first variant) and (ii) does not contain the first variant 130. For example, consider the case where the variant is a single nucleotide variant associated with a predetermined genomic location. In this instance, a sequence read 126 supports the variant when the first variant maps to the predetermined genomic location and contains this single nucleotide variant. In practice, to determine whether the first variant contains this single nucleotide variant, the sequence read also contains the 5′ and 3′ sequences that flank this single nucleotide variant in the genome of the species of the subject in order to map the sequence read to the genome to determine if it maps to the genomic location corresponding to the variant. Next, consider the case where the variant is the insertion of 38 bases into a particular gene. A sequence read will support this variant when the sequence read contains the 38 base insertion (as well as 5′ and 3′ regions that flank this insertion in the particular gene). In some instances, it is still possible for the sequence read to support this variant when it contains less than the entirety of the variant. For instance, the sequence read may terminate about 25 bases into the 38 base insertion. Nevertheless, the region of the sequence read flanking this insertion may match the gene and the first 25 bases of the insertion and thus sequence read can be deemed to support the variant. Further, a number of sequence reads 126 in the second plurality of sequence reads that support a first variant 130 in the reference set 128 versus a number of sequence reads 126 in the second plurality of sequence reads that do not support the first variant 130 determine the observed frequency (support 132) of the first variant 130. For example, consider again the case where a variant is associated with a particular genomic location in the genome, that the second plurality of reference sequence reads 126 from the first aberrant sample consist of 1000 sequence reads but that only 100 of these 1000 sequence reads cover (map to, are associated with) the genomic location associated with the variant. The 100 sequence reads that cover the genomic location associated with the variant are analyzed to see whether they support or do not support the variant. Those sequence reads in the 100 sequence reads that contain all or a portion of the variant are deemed to support the variant, and those sequence reads in the 100 sequence reads that do not contain the variant are deemed to not support the variant. The other 900 sequence reads do not qualify for supporting or not supporting the variant because they do not cover the genomic region associated with the variant in question. Further, consider the case where 3 of the 100 sequence reads contain all or a portion of the variant and are deemed to support the variant, and the remaining 97 sequence reads in the 100 sequence reads do not contain the variant and thus do not support the variant. In accordance with this embodiment, in this example the observed frequency (support 146) for the first variant is 3/100 or three percent.

Referring to block 256 of FIG. 2D, the method continues by evaluating the observed frequency of each respective variant in the first variant set 142 against the observed frequency of the respective variant in the first reference set 128 in the first aberrant solid tissue thereby determining a first tumor fraction in cell-free nucleic acid of the liquid biological sample of the subject.

In some embodiments, this first tumor fraction is used to classify a subject by deeming the subject to have a first condition when the observed frequency (support 14) of each variant 144 in the first variant set 142 satisfies a first threshold, where the first threshold is determined by a frequency of each variant 130 in the first reference set 128 in the first aberrant tissue sample. For instance, referring to block 258 of FIG. 2D, in some embodiments, the evaluating of block 256 comprises computing a single estimated ctDNA fraction in the cfDNA of the subject from the observed frequency (support 146) of each variant 144 in the first variant set 142 in the first plurality of sequence reads. Further, in such embodiments the first threshold is a single expected ctDNA fraction in the cfDNA of the subject that is determined from the frequency (reference frequency 132) of each variant 130 in the reference set 128 for the first aberrant tissue sample. For example, consider the case where a single variant is compared in the evaluating step of block 256. Thus, the support 146 for this variant in the variant set 142 from the biological sample (e.g., blood) is compared to the reference frequency 132 of the same variant in the reference set 128 for the aberrant tissue. The assumption is made that the sole source of the single variant in the cell-free nucleic acid arises from the aberrant tissue. Thus, with this assumption, the single estimated ctDNA fraction is computed as the ratio of the support 146 for the variant in the variant set 142 to the reference frequency 132 for the same variant in the reference set. For instance, if the support 146 for the variant is 3 out of 100 sequence reads in the variant set 142 and the reference frequency 132 of the same variant is 0.10 in the reference set 128, the single estimated ctDNA fraction is ( 3/100)/(0.10) or 0.3.

Next, consider the case in which two variants, a first variant and a second variant, are compared in the evaluating step of block 256. The support 146 for the first variant in the variant set 142 from the biological sample (e.g., blood) is compared to the reference frequency 132 of the same variant in the reference set 128 for the aberrant tissue. Likewise, the support 146 for the second variant in the variant set 142 from the biological sample is compared to the reference frequency 132 of the same variant in the reference set 128. The assumption is made that the sole source of the first and second variant in the cell-free nucleic acid arises from the aberrant tissue. Thus, with this assumption, a ratio for the first variant is calculated as the support 146 for the first variant in the variant set 142 to the reference frequency 132 for the first variant in the reference set. For instance, if the support 146 for the first variant is 3 out of 100 sequence reads in the variant set 142 and the reference frequency 132 of the first variant is 0.10 in the reference set 128, the ratio for the first variant is ( 3/100)/(0.10) or 0.3. Further, a ratio for the second variant is calculated as the support 146 for the second variant in the variant set 142 to the reference frequency 132 for the second variant in the reference set. For instance, if the support 146 for the second variant is 5 out of 85 sequence reads in the variant set 142 and the reference frequency 132 of the first variant is 0.12 in the reference set 128, the ratio for the second variant is (5/85)/(0.12) or 0.49.

In some embodiments, more than one variant is compared in the evaluating step of block 256 and a ratio between the observed support for each variant in the biological sample and the frequency of the same variant in the variant set is computed for each such variant. For example, in some embodiments, more than two variants are compared in the evaluating step of block 256. In such embodiments, the examples above are extended in the sense that a ratio between the observed support for each variant in the biological sample and the frequency of the same variant in the reference set is computed for each such variant. Indeed, in some embodiments, between two and 200 variants are compared in the comparing step of block 228. In some embodiments, more than 25, 50, 100, 200, 300, 400, 500, 1000, 2000, or 5000 variants are compared in the evaluating step of block 256.

Thus, a number of somatic variants k are observed from the first aberrant tissue sample, where k is a positive integer (e.g., 2, 3, more than 20, more than 100, more than 200, etc.). This can be expressed as a k-length vector f₁=(f₁₁, f₁₂, . . . , f₁ₖ) of variant frequencies (number of sequence reads 126 that support the variant au over the total number sequence reads 126 dₗᵢ mapping to the genomic location corresponding to the variant) for each variant in the reference set, where each component f₁ᵢ of f₁ takes a value between zero to one. This forms the reference set 128.

Further, sequence reads overlapping the k variants represented by the vector f1 are scanned from the biological sample comprising cell-free nucleic acid molecules from the subject. For each respective variant location i in the k variant locations, the total number of sequence reads 140 (d₂ᵢ) mapping to the genomic location corresponding to the variant location i (e.g., covering variant location i) and the number of these sequence reads 140 matching the variant (a₂ᵢ) is determined. The measurements d₂ᵢ and a₂ᵢ are non-negative integer values, from which a quotient f₂ᵢ is taken of a₂ᵢ by d₂ᵢ. The respective quotients f₂ᵢ for the variants across the reference set using the sequence reads 140 measured from the biological sample comprising cell-free nucleic acid modules from the subject can be expressed as the k-length vector f₂=(f₂₁, f₂₂, . . . , f₂ₖ) of variant frequencies (number of sequence reads 140 mapping to the genomic location represented by a given variant that match the given variant over the total number sequence reads 140 mapping to the genomic location represented by the given variant) for each variant in the reference set.

The objective is to determine a single estimated ctDNA fraction of the subject from the observed frequency (support 146) of each variant 144 in the first variant set 142 in the first plurality of sequence reads in accordance with block 256. In other words, the goal is to determine the single estimated ctDNA fraction, using the fraction of mutant reads contributed from the first aberrant tissue sample (e.g., tumor) to the biological sample comprising cell free nucleic acid (e.g., blood). The vectors f₁ and f₂ summarize the measured sequence read counts from the respective tissues (first aberrant tissue and biological sample containing cell free nucleic acid) from which the underlying rate is to be inferred. In some embodiments, variants that are clearly not associated with cancer are excluded from the analysis. In other words, they are excluded from the k variants considered.

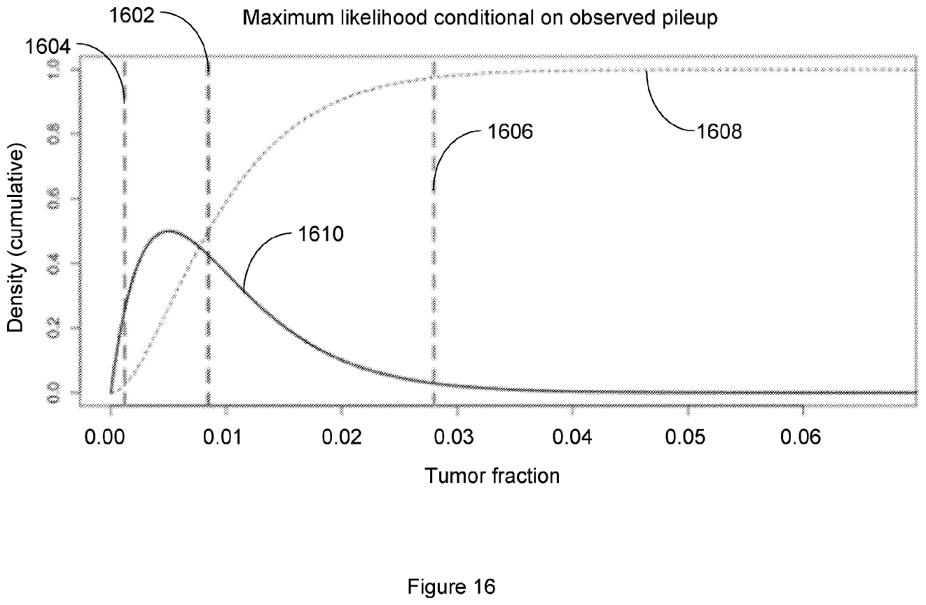

In some embodiments, it is assumed that the sequence reads 126 from the aberrant tissue sample are generated according a Poisson Process. For each variant i in k, there is observed a₂ᵢ actual supporting sequence read counts, and f₁ᵢ times d₂ᵢ expected supporting read counts. For example, for variant 1, consider the case where a₂₁ is 100 and d₂₁ is 1000 meaning that, of the 1000 sequence reads 140 measured from the biological sample containing cell-free nucleic acid that overlap the genomic location corresponding variant 1, 100 of the sequence reads 140 support the variant. Further suppose that, from the first aberrant tissue, it was determined that the frequency of this variant in the first aberrant tissue (f₁₁) is 0.25. It is expected, therefore, that there be f₁₁ (0.25) times d₂₁ (1000) or 250 read counts. Accordingly, in some embodiments a cumulative distribution function (binomial cumulative probability function) is estimated of the data conditional on t (the rate mutant sequence reads are contributed from the first aberrant tissue sample to the biological sample containing the cell free nucleic acid), D(t), to estimate single estimated ctDNA fractions corresponding to the 5^{th}, 50^{th} (median), and 95^{th} percentiles or any other desired percentiles. What is observed in the cell free DNA biological sample is a₂ᵢ supporting reads for a respective variant i in the k variants considered. Further, a calculation of how many sequence reads supporting the respective variant i in the k variants would be expected from the biological sample containing the cell free nucleic acid can be calculated as the variant frequency of the first aberrant tissue f₁ᵢ for the respective variant i in the first aberrant tissue sample multiplied by d₂ᵢ (the number of sequence reads mapping to the genomic position covering variant i observed in the biological sample containing the cell free nucleic acid) assuming a one hundred percent shed rate (meaning that the only source of contribution to the biological sample containing cell free nucleic acid (e.g., blood sample) is from the aberrant tissue). So, from this, t, which can be considered the fraction that converts (i) the expected number of reads supporting variant i (based on the analysis of the first aberrant tissue fraction f₁ᵢ) to (ii) the actual observed number of reads supporting variant i in the tissue containing cell free DNA (a₂ᵢ), can be calculated and introduced into a Poisson model and this can be used to estimate a cumulative distribution function (a probability distribution) that provides an estimate for each trial value of t (where t is sampled from anywhere between zero percent and 110 percent in some embodiments). Thus, referring to FIG. 16, the likelihood of the respective trial value of t for a given allele i is calculated using the cumulative distribution function across a range of values for t.

In some embodiments, the cumulative distribution function, for a single variant, has a value that ranges between 0 (zero probability) and 1 (one hundred percent probability) and has the form:

$$P ( x ; p , n ) = {\sum }_{i = 0}^{x} \frac{n !}{i ! ( n - i ) !} {( p )}^{i} {( i - p )}^{( n - i )}$$

where, x=a₂ᵢ, the number of sequence reads from the biological sample matching the genomic location corresponding to the variant i and that support the variant allele at that location, p=t*f₁ᵢ, where t is the single estimated ctDNA fraction, and f₁ᵢ is the ratio of (a) the number of sequence reads from the first aberrant tissue matching the genomic location corresponding to the variant i and that support the variant allele at that location, and n=d₂ᵢ, the total number of sequence reads from the biological sample mapping to the genomic location corresponding to the variant location i.

From this, and referring to FIG. 16, the median value for t (the most likely value for t) based on the distribution of likelihoods for t across the range of values of 0 percent to 110 percent for t (1602), the 5^{th} percentile value for t (lowest value for t, lower bound for t) based on the distribution of likelihoods for t across the range of values of 0 to 110 percent for t (1604), and the 95^{th} percentile (highest value for t, upper bound for t) value for t based on the distribution of likelihoods fort across the range of values of 0 to 110 percent fort (1606), can be calculated. In FIG. 16, the solid line 1610 represents the cumulative density function whereas the line 1608 represents the cumulative distribution function. The cumulative distribution function is used to compute the percentile values for t in some embodiments. The 95^{th} percentile value means that an observed fraction of sequence reads supporting a variant allele over the total number of sequence reads overlapping the allele position exceeding the 95^{th} percentile value for t is extremely rare and 95 percent of the time a value for t less than the 95^{th} percentile value for t (about 28 percent in FIG. 16) is expected.

Other bounds, such as the 2^{nd} percentile and 98^{th} percentile, can be used.

The above discussion relates to how t is calculated from a single variant. However, as discussed herein, in more common embodiments, multiple variants are sampled, and thus each variant produces an independent likelihood (probability for t) across the range of values (e.g., 0 to 100 percent) considered for t. Thus, the cumulative distribution function provides a first probability for t at a given trial value of t based on the observed and expected values for variant 1, a second probability for t at the given trial value of t based on the observed and expected values for variant 2, and so forth. To arrive at the cumulative likelihood for t at the given trial value of t, each of the component probabilities (the first probability for t at the given trial value of t based on the observed and expected values for variant 1, the second probability for t at the given trial value of t based on the observed and expected values for variant 2, and so forth) are combined and used to compute the cumulative distribution function. In other words, the cumulative distribution function 1608 of FIG. 16 can be drawn using the data from any number of variants based on the assumption that they are independent observations of the same underlying single estimated ctDNA fraction. In some embodiments, the probabilities provided by each respective variant in the set of k variants for a given trial value of t are combined by adding them together when the probabilities are expressed in logarithmic space to arrive at the computed probability of the trial value for t. For instance:

$$log P ( {x}_{k} ; {p}_{k} , {n}_{k} ) = {\sum }_{k} log ( {\sum }_{i = 0}^{x} \frac{{n}_{k} !}{i ! ( {n}_{k} - i ) !} {( {p}_{k} )}^{i} {( i - {p}_{k} )}^{( {n}_{k} - i )} )$$

where k refers to the k^{th} allele and the summation is over all k variants. Alternatively, in some embodiments, the probabilities provided by each respective variant in the set of k variants for a given trial value of t are combined by multiplying them together when the probabilities are expressed in natural scale to arrive at the computed probability of the trial value for t.

In some embodiments, the Poisson model of the likelihood of t across the trial range of t is computed individually for each variant k thereby computing a plurality of Poisson models, one for each variant. Then the plurality of Poisson models is combined (e.g., summed in log space or multiplied if on the natural scale) for each trial value of t sampled, in order to obtain the likelihood of a trial value of t for each trial value of t sampled. As such, each point in line 1608 is aggregated across the k variants, where k is a positive integer (e.g., 2 or more, 20 or more, 1000 or more). In this way, the most parsimonious explanation of tumor fraction is provided.

In some embodiments, the single estimated ctDNA fraction is taken as the median value for t taken from the distribution of likelihoods for t across the range of values of t sampled using the cumulative density function.

Importantly, this framework enables confidence intervals to be estimated on single estimated ctDNA fractions in instances in which zero supporting reads 140 are observed in the biological sample over the k variants.

As such, the cell free DNA tumor fraction is estimated conditional on the read information for the set of variants between the (i) biological sample containing the cell free nucleic acid and (ii) the first aberrant tissue sample. In this embodiment, therefore, only those variants that are represented in both the reference set of variants 128 and the variant set for the biological sample 142 are used to compute the single estimated ctDNA fraction of the subject.

In alternative embodiments, a negative binomial distribution assumption is assumed rather than a Poisson distribution in order to compute the cumulative distribution function 1608 of FIG. 16.

In some embodiments, observed sequence reads are corrected for background copy number. For instance, sequence reads that support variants that arise from chromosomes or portions of chromosomes that are duplicated in the subject are corrected for this duplication. This can be done either by normalizing before running this inference, or allowing for more than one value of ctDNA fraction. Allowing for more than one ctDNA fraction also enables assessment of heterogeneity within/across tumors. As such, in some embodiments, the assumption that each variant represents an independent observation of the single estimated ctDNA fraction is corrected for background copy number.

As another example referring to FIG. 3, discussed above, in some embodiments, the single expected ctDNA fraction in the cfDNA is between 0.5×10⁻⁴ and 1.5×10⁻⁴, and the first condition is a melanoma. In some embodiments, the single expected ctDNA fraction in the cfDNA is between 0.5×10⁻³ and 1×10⁻², and the first condition is a renal cancer, uterine cancer, thyroid cancer, prostate cancer, breast cancer, bladder cancer, gastric cancer, cervical cancer or a combination thereof. In some embodiments, the single expected ctDNA fraction in the cfDNA is between 1×10⁻² and 0.8, and the first condition is lung cancer, esophageal cancer, a head/neck cancer, colorectal cancer, anorectal cancer, ovarian cancer, a hepatobiliary cancer, a pancreatic cancer, or a lymphoma.

In some embodiments, a subject is classified by deeming the subject to have a first condition when the observed frequency (support 14) of each variant 144 in the first variant set 142 satisfies a first threshold. In some embodiments, the first threshold is determined based on a quantification of the reference frequency for the variants in the variant set. In some embodiments, for instance, the observed frequency (support 14) of each variant 144 in the first variant set 142 is normalized by the reference frequency for the corresponding variants in the variant set as discussed above with reference to block 258 in order to realize a circulating tumor nucleic acid fraction for the subject. For instance, in some embodiments, the observed frequency (support 14) of each variant 144 in the first variant set 142 is divided by the reference frequency for the corresponding variants in the variant set as discussed above with reference to block 258 in order to realize the circulating tumor nucleic acid fraction for the subject. In this way, the first threshold is determined by a frequency of each variant 130 in the first reference set 128 in the first aberrant tissue sample.

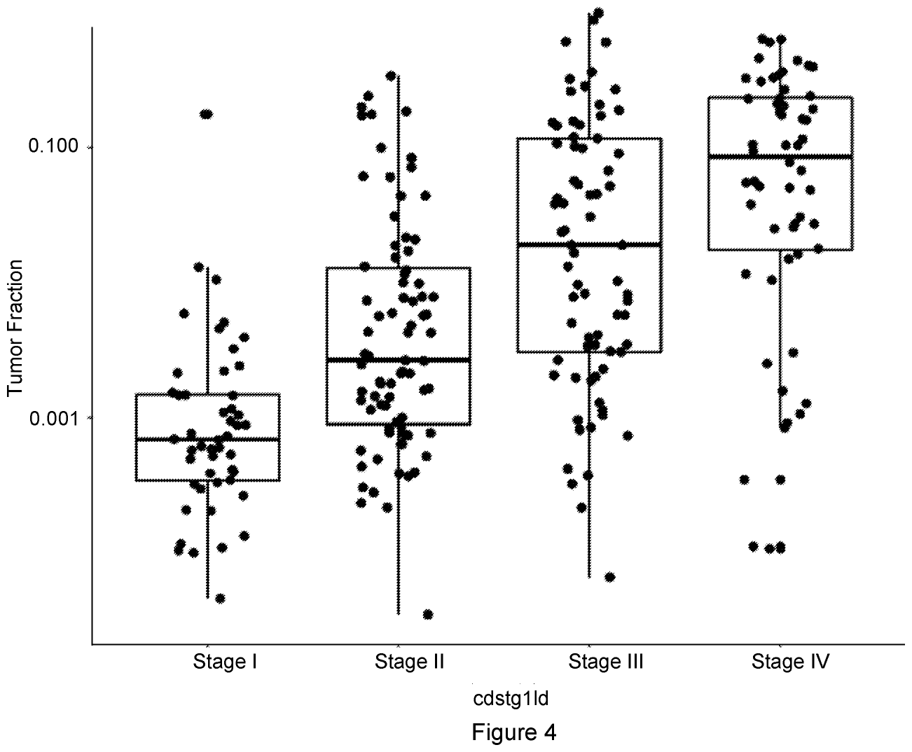

In some embodiments, a cohort of subjects with a similar condition is used to refine the first threshold value associated with a condition. For example, consider the case where the first condition is stage of cancer, irrespective of the type of cancer. FIG. 4 illustrates the shedding rates (ctDNA fraction) across a cohort of subjects. Each point in FIG. 4 represents the ctDNA fraction of a different subject in a cohort of subjects broken out into one of four cancer stages (I, II, III, and IV). For each respective subject, the ctDNA fraction (tumor fraction) is plotted as the ratio of the support for the set of variants in the variant set 142 collected from a biological sample of the subject (e.g., in accordance with blocks 202 and 210 of FIG. 2) and the reference frequencies 132 for these same variants in the reference set 128 for the respective subject obtained from a tumor from the same subject (e.g., in accordance with the disclosure outlined for block 228 of FIG. 2). FIG. 4 illustrates that there is a range of ctDNA fraction values for each cancer subject but that the median ctDNA fraction value generally increases with increased cancer stage. FIG. 4 thus provides motivation for determining the first threshold based on a quantification of the reference frequency for the variants in the variant set. That is, FIG. 4 illustrates the potential for using observed frequencies of variants in the aberrant tissue of a given subject, and optionally information regarding expected ctDNA fraction for subjects having a particular phase or type of cancer, to determine a first threshold for the given cancer subject that can be evaluated against the observed frequency of the variants in a variant set for a biological sample of the given subject in order to classify the subject as having or not having the condition (e.g., a clinical stage of a given cancer). Thus, referring to FIG. 4, a first threshold of 0.05 can be used to analyze whether a subject has stage I of a given cancer. In such a case an aberrant tissue, such as a tumor, is obtained from a subject and used to determine a reference frequency for each respective variant in a first reference set (e.g., in accordance block 228 of FIG. 2). In fact, in some embodiments, the frequency of various possible variants is used to identify the variants for the reference set. Further, cell free nucleic acid is obtained from a biological sample, other than the aberrant tissue, of the same subject (e.g., in accordance with block 202) and the variant frequency of the same variants that are in the reference set are determined from sequence reads of the cell free nucleic acids in the biological sample (e.g., in accordance with block 210). The variant frequency (support 146) of these variants in the biological sample are normalized by the reference frequency of the same variants in the aberrant tissue (e.g., by taking a ratio, etc.) to form the observed ctDNA fraction of the biological sample (e.g., in accordance with the disclosure of block 258 of FIG. 2). Here, the first threshold is determined by a frequency of each variant 130 in the first reference set 128 in the first aberrant tissue sample because these frequencies form the basis of the denominator of the ratio as discussed above in conjunction with block 258 of FIG. 2. That is, in this example, the frequency of each variant 130 in the first reference set 128 in the first aberrant tissue sample determines the first threshold because they are used as a basis for calculating the ctDNA fraction of the subject. Using the cohort of FIG. 4 as a guide, the determination of whether the ctDNA fraction for a given biological sample satisfies the threshold condition of 0.05 provides the basis for determining whether or not the subject has stage I cancer in this example. For instance, from FIG. 4, when the comparison of the observed frequencies (support 146) of each variant 144 in the variant set to the reference frequencies of the same variants in the reference set 128 indicates that the ctDNA fraction is more than 0.05, the subject is deemed to have a more advanced stage of cancer because very few stage I subjects in the cohort of FIG. 4 have a ctDNA fraction that is more than 0.05. On the other hand, observation of a ctDNA fraction that is less than 0.001 is consistent with a finding that the subject has stage I of a given cancer because relatively few stage II, III, or IV subjects in the cohort of FIG. 4 have a ctDNA fraction that is less than 0.001. This is just one example, and FIG. 3, discussed in greater detail in Example 1 below, shows that more precise threshold values can be defined when the type of cancer that a subject has is already known.

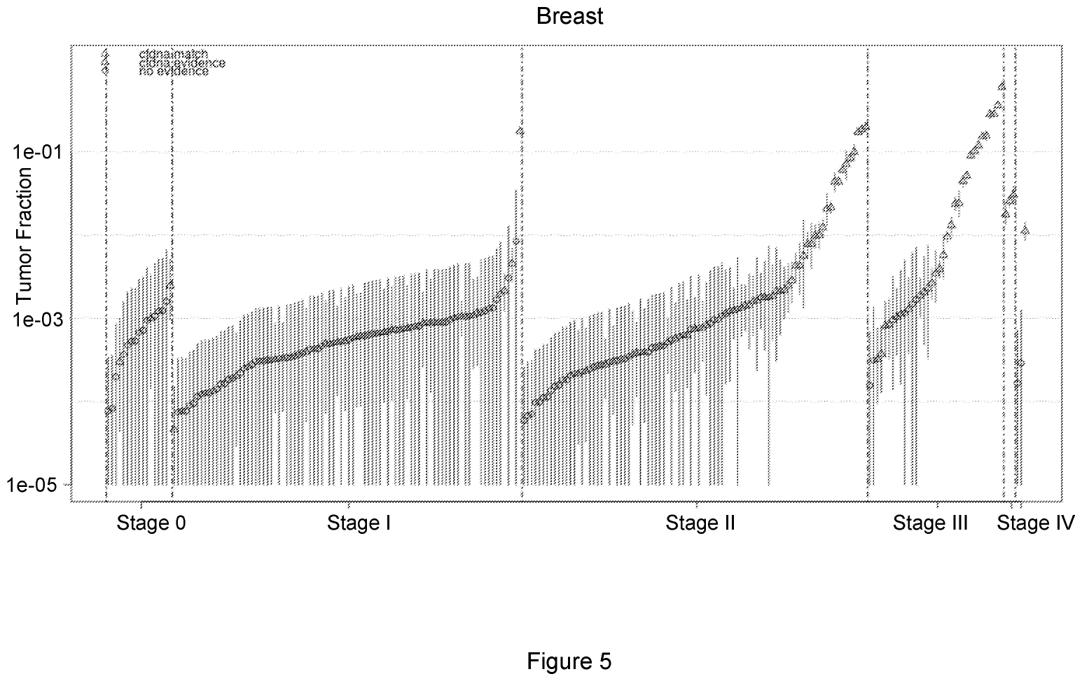

Block 260 provides a specific embodiment in which the evaluating of block 256 comprises computing a single estimated circulating tumor DNA (ctDNA) fraction in the cell free DNA (cfDNA) of the subject from the observed frequency (support 146) of each variant 144 in the first variant set 142, where the observed frequency of each first variant 144 in the first variant set 142 satisfies a threshold when the single estimated circulating tumor DNA (ctDNA) fraction exceeds 1×10⁻³, and the first condition is stage II, stage III, or stage IV breast cancer. This threshold limit is supported by FIG. 5, discussed in Example 2 below. In FIG. 5, each point is the ctDNA fraction of an individual subject that has breast cancer. The method used in some embodiments to compute the cfDNA fraction for each subject comprises obtaining a first plurality of sequence reads 140 in electronic form from a biological sample of each subject in a cohort, where the biological sample comprises cell-free nucleic acid molecules. The first plurality of sequence reads 140 are used to identify support for each variant 144 in a variant set 142 for the biological sample thereby determining an observed frequency (support 146) of each variant 144 in the variant set 142. In some embodiments, the observed frequency (support 146) of each respective variant 144 in the variant set 142 is compared to a corresponding reference frequency 132 for the respective variant in a reference set 128. Each such corresponding reference frequency 132 in the reference set 128 is a frequency of a respective variant in a first aberrant tissue sample obtained from the subject. In this way, the ctDNA fraction of each subject is determined in some embodiments. In addition to plotting the ctDNA fraction of each subject, FIG. 5 breaks the subjects out by stage of breast cancer. FIG. 5 indicates a very large dynamic range for tumor fraction that is observed within each tumor stage. FIG. 5 indicates that if the circulating tumor DNA (ctDNA) fraction exceeds 1×10⁻³, it is possible that the subject has stage II, III, or IV breast cancer since very few stage 0 or stage I breast cancer subjects in FIG. 5 have a ctDNA fraction exceeding 1×10^{-3.} Of course, additional tests may be needed to determine the exact classification of a breast cancer subject since FIG. 5 also shows that a substantial number of stage III subjects have ctDNA fractions below 1×10⁻³. As such the disclosed methods support instances where the subject has stage II, stage III, or stage IV breast cancer and the evaluating of block 256 determines that the first tumor fraction of the cell-free nucleic acid is less than 1×10⁻³.

Referring to block 262, in some embodiments the disclosed methods are used to evaluate a tumor fraction in a subject that has a cancer from a common primary site of origin. For instance referring to block 264, in some embodiments, the disclosed methods are used to evaluate a tumor fraction in a subject that has breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

Referring to block 268 of FIG. 2E, in some embodiments the disclosed methods are used to evaluate a tumor fraction in a subject that has a predetermined stage of a breast cancer, lung cancer, prostate cancer, colorectal cancer, renal cancer, uterine cancer, pancreatic cancer, esophagus cancer, lymphoma, head/neck cancer, ovarian cancer, hepatobiliary cancer, melanoma, cervical cancer, multiple myeloma, leukemia, thyroid cancer, bladder cancer, or gastric cancer.

Referring to block 270 of FIG. 2E, in some embodiments, the disclosed methods are used to evaluate a tumor fraction in a subject that has a predetermined subtype of a cancer. In some such embodiments, referring to block 272 of FIG. 2E, the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

Referring to blocks 274 through 286 of FIGS. 2E and 2F, the disclosed methods are not limited to the analysis of a single aberrant tissue or to the analysis of a single aberrant tissue at a single time point. In some embodiments, the disclosure of block 202 through 272 is extended to multiple tumor samples and multiple tumor fractions in one patient corresponding to inter/intra tumor heterogeneity. In other words, the disclosed methods can be used to calculate an additional ctDNA fraction of a second biological sample with respect to a second aberrant tissue. For instance, in some such embodiments, the first aberrant tissue sample discussed above in conjunction with blocks 202 through 272 of FIG. 2 is of a first cancer type and the second aberrant tissue sample is of a second cancer type (block 278). In other embodiments, the first aberrant tissue sample discussed above in conjunction with blocks 202 through 272 of FIG. 2 is from a tumor at a first time point, and the second aberrant tissue sample is from the same tumor at a second time point. In still other embodiments, the aberrant tissue in the subject is heterogeneous and the first aberrant tissue sample is a first section of this aberrant tissue and the second aberrant tissue sample is a second section of this same aberrant tissue collected at the same time as the first section.

In more detail, referring to block 274, the first plurality of sequence reads 140 is used to identify support for each variant 144 in a second variant set 142 thereby determining an observed frequency of each variant 144 in the second variant set. For each respective variant 144 in the second variant set 142, a corresponding reference frequency 132 for the respective variant is obtained in a second reference set 128, where each corresponding reference frequency in the second reference set is for a respective variant in a second aberrant solid tissue sample obtained from the subject. In such embodiments, the evaluating of block 256 further comprises using the observed frequency of each respective variant in the second variant set against the observed frequency of the respective variant in the second reference set, thereby determining a second tumor fraction in cell-free nucleic acid of the liquid biological sample of the subject. In this way, the ctDNA fraction of a biological sample can be first calculated with respect to the first aberrant tissue (e.g., to determine whether the subject has a first condition, to monitor progression of the first aberrant tissue over time, to monitor tumor heterogeneity, etc.) and a different ctDNA fraction of the biological sample can be calculated with respect to the second aberrant tissue (e.g, to determine whether the subject has a second condition, to monitor progression of the second aberrant tissue over time, to monitor tumor heterogeneity, etc.).

Referring to block 276 of FIG. 2F, in some embodiments, a respective sequence read 140 in the first plurality of sequence reads is deemed to support a variant 144 in the second variant set 142 when the respective sequence read 140 (i) maps onto to genomic position corresponding to the variant and (ii) contains all or a portion of the variant 144. A respective sequence read 140 in the first plurality of sequence reads is deemed to not support a variant 144 in the second variant set 142 when the respective sequence read 140 (i) maps onto to genomic position corresponding to the variant and (ii) does not contain the variant 144.

Referring to block 278 of FIG. 2F, in some embodiments, the first aberrant tissue sample consists of a first tumor fraction and the second aberrant tissue sample consists of a second tumor fraction of a common (same) tumor from the subject.

Referring to block 280 of FIG. 2F, in some embodiments, the first aberrant tissue sample is of a first cancer type and the second aberrant tissue sample is of a second cancer type. The first cancer type can be the same as the second cancer type (block 282). Alternatively, the first cancer type can be different than the second cancer type (block 284). In some embodiments, the first cancer type and the second cancer type are each selected from the group consisting of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, and gastric cancer (block 286).

Exemplary Method Embodiment—Evaluating an aggressiveness of a known condition in a subject based on variation in ctDNA fraction in cfDNA over time.

Another aspect of the present disclosure provides a method of evaluating a state of a condition in a subject. The method comprises, at a computer system 100 having one or more processors 102 and memory 111/112 storing one or more programs for execution by the one or more processors, obtaining in electronic form, for each respective time point in a plurality of time points across an epoch, from a respective biological sample of the subject taken at the respective time point, a corresponding dataset 138 comprising a corresponding first plurality of sequence reads 140 of the respective biological sample, thereby obtaining a plurality of datasets of the subject (e.g., as set forth in block 202). Each respective biological sample comprises cell-free nucleic acid molecules. In some embodiments, the cell-free nucleic acid molecules from a particular biological sample are obtained as discussed above in conjunction with any of blocks 202 through 208 of FIG. 2. In some embodiments, the sequence reads for the cell-free nucleic acid molecules of a particular biological sample are obtained as discussed above in conjunction with any of blocks 202 through 208 of FIG. 2.

The method further comprises determining, for each respective dataset (e.g., data construction 138) in the plurality of respective datasets, support for each variant 144 in a variant set 142 (e.g., as disclosed in blocks 210 through 226 of FIG. 2). A respective sequence read 140 in the first plurality of sequence reads of the respective dataset is deemed to support a variant 144 in the variant set 142 when the respective sequence read 140 (i) maps to a genomic location corresponding to the variant and (ii) contains all or a portion of the variant 144. A respective sequence read 140 in the first plurality of sequence reads of the respective dataset is deemed to not support a variant in the variant set when the respective sequence read (i) maps to a genomic location corresponding to the variant and (ii) does not contain all or a portion of the variant. In this way, an observed frequency of each variant 144 in the variant set 142 is determined using the sequence reads 140 in the first plurality of sequence reads of the respective dataset 138 that do support and do not support each variant 144 in the variant set 142 at each time point in the plurality of time points.

In some embodiments, the sequence reads 140 are used to find support for variants 144 in the variant set 142 by using the sequence reads 140 to call variations using the B score classifier. The B score classifier is described in U.S. Patent Publication No. 62/642,461, entitled “Method and System for Selecting, Managing, and Analyzing Data of High Dimensionality,” filed Mar. 13, 2018, which is hereby incorporated by reference, and which is described in further detail in Example 3.

In some embodiments, the sequence reads 140 are used to find support for variants 144 in the variant set 142 by using the sequence reads 140 to call variations using the M score classifier. The M score classifier is described in U.S. Patent Application No. 62/642,480, entitled “Methylation Fragment Anomaly Detection,” filed Mar. 13, 2018, which is hereby incorporated by reference.

In some embodiments, the sequence reads 140 are used to find support for variants 144 in the variant set 142 by using the sequence reads 140 to call variations using the techniques disclosed in any of blocks 210 through 216 described above in conjunction with FIG. 2.

The method further comprises evaluating the observed frequency (e.g., support 146) of each variant 144 in the variant set 142 at each time point in the plurality of time points against the observed frequency of the respective variant in the first aberrant solid tissue (e.g., as determined in the first instance of block 210) to determine the state or progression of a disease condition in the subject during the epoch in the form of an increase or decrease of the first tumor fraction over the epoch.

In some embodiments, the epoch is calibrated for an ability to measure changes in ctDNA on the order of hours (e.g., to measure surgery success in removing aberrant tissue from a subject), weeks/months (e.g., to monitor success of therapy for a subject), or years (e.g., to monitor for disease remission in a subject). Thus, in some embodiments, the epoch is a period of months and each time point in the plurality of time points is a different time point in the period of months. In some such embodiments, the period of months is less than four months. In some embodiments, the epoch is a period of years and each time point in the plurality of time points is a different time point in the period of years. In some such embodiments, the period of years is between two and ten years. In some embodiments, the epoch is a period of hours and each time point in the plurality of time points is a different time point in the period of hours. In some such embodiments, the period of hours is between one hour and six hours.

In some embodiments, the evaluating the observed frequency of each variant 144 in the variant set 142 at each time point in the plurality of time points against the observed frequency of the respective variant in the first aberrant solid tissue comprises computing a respective single estimated circulating tumor DNA (ctDNA) fraction in the cell free DNA (cfDNA) of the subject from the observed frequency of each variant 144 in the variant set 142 at each time point in the set of time points in the manner set forth in conjunction with block 256 above. In some such embodiments, the method further comprises changing a diagnosis of the subject when the respective single estimated ctDNA fraction in the cfDNA of the subject is observed to change by a threshold amount across the epoch. For instance, in some embodiments the ctDNA fraction at each time point in the epoch is a number between 0 and 1 and, when the ctDNA fraction changes by a predetermined amount during the epoch, the diagnosis of the subject is changed. In one example, when the ctDNA fraction increases more than two percent, more than three percent, more than four percent, more than five percent, more than ten percent or more than twenty percent during the epoch, the diagnosis of the subject is downgraded, indicating that the subject has a more aggressive form of the disease condition and/or a later stage of the disease condition than initially diagnosed. In another example, when the ctDNA fraction decreases more than two percent, more than three percent, more than four percent, more than five percent, more than ten percent or more than twenty percent during the epoch, the diagnosis of the subject is upgraded, indicating that the subject has a less aggressive form of the disease condition and/or an earlier stage of the disease condition than initially diagnosed.

In some embodiments, the method further comprises changing a prognosis of the subject when the respective single estimated ctDNA fraction in the cfDNA of the subject is observed to change by a threshold amount across the epoch. For instance, in some embodiments the ctDNA fraction at each time point in the epoch is a number between 0 and 1 and, when the ctDNA fraction changes by a predetermined amount during the epoch the prognosis of the subject is changed. In one example, when the ctDNA fraction increases more than two percent, more than three percent, more than four percent, more than five percent, more than ten percent or more than twenty percent during the epoch, the prognosis of the subject is downgraded, indicating that the likelihood of recovery of the subject from the disease condition decreases. In another example, when the ctDNA fraction decreases more than two percent, more than three percent, more than four percent, more than five percent, more than ten percent or more than twenty percent during the epoch, the prognosis of the subject is upgraded, indicating that the likelihood of recovery of the subject from the disease condition improves.

In some embodiments, the method further comprises changing a treatment of the subject when the respective single estimated ctDNA fraction in the cfDNA of the subject is observed to change by a threshold amount across the epoch. For instance, in one example, when the ctDNA fraction increases more than two percent, more than three percent, more than four percent, more than five percent, more than ten percent or more than twenty percent during the epoch, the treatment regimen of the subject is changed to a more aggressive treatment. In another example, when the ctDNA fraction decreases more than two percent, more than three percent, more than four percent, more than five percent, more than ten percent or more than twenty percent during the epoch, the treatment regimen of the subject is changed to a less aggressive treatment.

In some embodiments, the condition is a disease, such as cancer. For instance, in some embodiments the disease is a cancer, and the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof.

In some embodiments, the condition is a stage of a breast cancer, lung cancer, prostate cancer, colorectal cancer, renal cancer, uterine cancer, pancreatic cancer, esophagus cancer, lymphoma, head/neck cancer, ovarian cancer, hepatobiliary cancer, melanoma, cervical cancer, multiple myeloma, leukemia, thyroid cancer, bladder cancer, or gastric cancer.

In some embodiments, the disease condition is a predetermined subtype of a cancer, where the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

In some embodiments, each respective variant in the variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

In some embodiments, the aberrant tissue is a tumor. In some embodiments, the first aberrant tissue sample is one of the aberrant tissues described above with reference to block 230 of FIG. 2.

In some embodiments, the variant set 142 consists of a single variant 144 that is a single genetic variation at a single locus in the genome of the subject. In some embodiments, the variant set 142 consists of a first variant that is a first genetic variation at a first locus in the genome of the subject and a second variant that is a second genetic variation at a second locus in the genome of the subject.

In some embodiments, the variant set 142 consists of a first variant 144 that is a first genetic variation at a first locus in the genome of the subject, a second variant 144 that is a second genetic variation at a second locus in the genome of the subject, and a third variant 144 that is a third genetic variation at a third locus in the genome of the subject.

In some embodiments, the variant set 142 consists of between two and twenty variants 144, where each variant 144 in the variant set is a different genetic variation (and optionally at a different locus) in the genome of the subject. In some embodiments, the variant set 142 comprises 30 variants 144, 50 variants 144, 75 variants 144, 100 variants 144, 125 variants 144, 250 variants 144, 500 variants 144, 750 variants 144, 1000 variants 144, 2500 variants 144, or 5000 variants 144, where each variant 144 in the variant set is a different genetic variation (and optionally at a different locus) in the genome of the subject.

In some embodiments, the determining, for each respective dataset in the plurality of respective datasets, support for each variant 144 in a variant set 142 comprises aligning a sequence read 140 in the first plurality of sequence reads of a respective dataset to a region in a reference genome in order to determine whether the sequence read contains all or a portion of a variant in the variant set. See, for example, block 212 of FIG. 2A and the disclosure for the same presented above.

In some embodiments, the determining, for each respective dataset in the plurality of respective datasets, support for each variant 144 in a variant set 142 comprises aligning a sequence read 140 in the first plurality of sequence reads of a respective dataset to a lookup table of variants in order to determine whether the sequence read contains all or a portion of a variant in the variant dataset. See, for example, block 214 of FIG. 2A and the disclosure for the same presented above.

In some embodiments, the determining, for each respective dataset in the plurality of respective datasets, support for each variant 144 in a variant set 142 comprises aligning a sequence read 140 in the first plurality of sequence reads of a respective dataset to each entry in a lookup table, where each entry in the lookup table represents a different portion of a reference genome. See, for example, block 216 of FIG. 2A and the disclosure for the same presented above.

In some embodiments, the subject is a human subject. In some embodiments, the subject is a mammalian. In some embodiments the subject is any of the species disclosed above in conjunction with block 204 of FIG. 2.

In some embodiments, the respective biological sample comprises blood, whole blood, plasma, serum, urine, cerebrospinal fluid, fecal, saliva, sweat, tears, pleural fluid, pericardial fluid, and/or peritoneal fluid of the subject. That is, the biological sample is a mixture of blood, whole blood, plasma, serum, urine, cerebrospinal fluid, fecal, saliva, sweat, tears, pleural fluid, pericardial fluid, and/or peritoneal fluid of the subject and one or more other components of the subject.

In some embodiments, the respective biological sample consists of blood, whole blood, plasma, serum, urine, cerebrospinal fluid, fecal, saliva, sweat, tears, pleural fluid, pericardial fluid, and/or peritoneal fluid of the subject. That is, the biological sample is blood, whole blood, plasma, serum, urine, cerebrospinal fluid, fecal, saliva, sweat, tears, pleural fluid, pericardial fluid, and/or peritoneal fluid of the subject and no other components of the subject.

Exemplary Method Embodiment—Using tumor fraction to gate usage of the results of a classifier

Another aspect of the present disclosure provides a method of classifying a subject. The method comprises, at a computer system 100 having one or more processors 102, and memory 111/112 storing one or more programs for execution by the one or more processors (e.g., condition monitoring module 120), obtaining in electronic form a dataset (e.g., data construct 138) comprising a first plurality of sequence reads 140 from a biological sample of the subject. Here, the biological sample comprises cell-free nucleic acid molecules. In some embodiments, the first plurality of sequence reads is obtained in any of the ways disclosed in conjunction with blocks 202 through 208. Moreover, in such embodiments, the first plurality of sequence reads 140 is used to identify support 146 for each variant 144 in a first variant set 142 thereby determining an observed frequency of each variant in the first variant set in the manner disclosed above with reference to any of blocks 210 through 226 disclosed above in conjunction with FIG. 2. Moreover, for each respective variant 144 in the first variant set 132, there is obtained a corresponding reference frequency 132 for the respective variant in a first reference set 128, where each corresponding reference frequency in the first reference set is for a respective variant in a first aberrant solid tissue sample obtained from the subject, in the manner disclosed above with reference to blocks 228 through 248 of FIG. 2. The method further discloses evaluating the observed frequency of each respective variant in the first variant set 142 against the observed frequency of the respective variant in the first reference set 128 in the first aberrant solid tissue thereby determining a first tumor fraction in cell-free nucleic acid of the liquid biological sample of the subject in any manner disclosed above with reference to blocks 256 through 272 of FIG. 2.

The method further comprises applying the first plurality of sequence reads (or dimension reduced data from the sequence reads, such as principal components) to a classifier thereby obtaining a classifier result. The classifier result indicates whether the subject has a first cancer condition. Further, prior to execution of the instant method, the classifier is trained on data other than observed tumor fraction data in the cell free DNA (cfDNA) of subjects. In some embodiments, the trained classifier result is used as a basis for diagnosis or prognosis of the subject for the first cancer condition when the first tumor fraction is between 0.003 and 1.0 and the trained classifier result indicates that the subject has the first cancer condition. As used herein, the term “trained classifier” refers to a model (e.g., a machine learning algorithm, such as logistic regression, neural network, regression, support vector machine, clustering algorithm, decision tree etc.) with fixed (locked) parameters (weights) and thresholds, ready to be applied to previously unseen samples.

In some embodiments, the estimated tumor fraction in the cfDNA of the subject is determined using the techniques disclosed above in reference to FIG. 2 and the Examples below.

In some embodiments, the first cancer condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof).

In some embodiments, the first cancer condition is a subtype of a cancer. In some such embodiments, the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

In some embodiments, the estimated tumor fraction is between 0.003 and 1.0 and the first cancer condition is a tissue of origin of a cancer.

In some embodiments, the computing the estimated tumor fraction in the cfDNA comprises using the dataset to identify support for each variant 144 in a variant set 142, where a respective sequence read 140 in the first plurality of sequence reads is deemed to support a variant 144 in the variant set 142 when the respective sequence read 140 (i) maps onto the portion of the genome corresponding to the variant and (ii) contains all or a portion of the variant 144, and a respective sequence read 140 in the first plurality of sequence reads is deemed to not support a variant 144 in the variant set 142 when the respective sequence read 140 (i) maps onto the portion of the genome corresponding to the variant and (ii) does not contain the respective variant 144. In this way, an observed frequency of each variant 144 in the variant set 142 is determined from among the sequence reads 140 in the first plurality of sequence reads that do support and do not support each variant in the variant set.

In some embodiments, the sequence reads 140 are used to find support for variants 144 in the variant set 142 by using the sequence reads 140 to call variations using the B score classifier. The B score classifier is described in U.S. Patent Publication No. 62/642,461, entitled “Method and System for Selecting, Managing, and Analyzing Data of High Dimensionality,” filed 62/642,461, which is hereby incorporated by reference, and which is described in further detail in Example 3.

In some embodiments, the sequence reads 140 are used to find support for variants 144 in the variant set 142 by using the sequence reads 140 to call variations using the M score classifier. The M score classifier is described in U.S. Patent Application No. 62/642,480, entitled “Methylation Fragment Anomaly Detection,” filed Mar. 13, 2018, which is hereby incorporated by reference.

In some embodiments, the sequence reads 140 are used to find support for variants 144 in the variant set 142 by using the sequence reads 140 to call variations using the techniques disclosed in any of blocks 210 through 216 described above in conjunction with FIG. 2.

Further, in such embodiments, a single estimated tumor fraction in the cfDNA of the subject is computed from the observed frequency of each variant in the variant set. See, for example, the disclosure of block 258 of FIG. 2 for disclosure on computing the single estimated tumor fraction in the cfDNA.

In some such embodiments, a variant in the variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

In some embodiments, the aberrant tissue sample is all or a portion of a tumor. In some embodiments, the aberrant tissue sample is any of the aberrant tissues described above in conjunction with block 230.

In some embodiments, the variant set 142 consists of a single variant 144 that is a single genetic variation at a single locus in the genome of the subject.

In some embodiments, the variant set 142 consists of a first variant 144 that is a first genetic variation at a first locus in the genome of the subject and a second variant 144 that is a second genetic variation at a second locus in the genome of the subject.

In some embodiments, the variant set 142 consists of a first variant 144 that is a first genetic variation at a first locus in the genome of the subject, a second variant 144 that is a second genetic variation at a second locus in the genome of the subject, and a third variant 144 that is a third genetic variation at a third locus in the genome of the subject.

In some embodiments, the variant set 142 consists of between two and twenty variants, where each variant 144 in the variant set 142 is a different genetic variation (and optionally at a different locus) in the genome of the subject. In some embodiments, the variant set comprises 40 variants, 50 variants, 75 variants, 100 variants, 200 variants, 500 variants, 1000 variants, 2000 variants, or 5000 variants, and each variant in the variant set is for a different genetic variation (and optionally at a different locus) in the genome of the subject.

In some embodiments, the single estimated tumor fraction in the cfDNA is between 0.5×10⁻⁴ and 1.5×10⁻⁴, and the first cancer condition is a melanoma. In some embodiments, the single estimated tumor fraction in the cfDNA is between 0.5×10⁻³ and 1×10⁻², and the first cancer condition is a renal cancer, uterine cancer, thyroid cancer, prostate cancer, breast cancer, bladder cancer, gastric cancer, cervical cancer or a combination thereof. In some embodiments, the single estimated tumor fraction in the cfDNA is between 1×10⁻² and 0.8, and the first cancer condition is lung cancer, esophageal cancer, a head/neck cancer, colorectal cancer, anorectal cancer, ovarian cancer, a hepatobiliary cancer, a pancreatic cancer, a lymphoma, or a combination thereof.

In some embodiments the using the first plurality of sequence reads to identify support for each variant in a variant set comprises aligning a respective sequence read 140 in the first plurality of sequence reads to a region in a reference genome in order to determine whether the respective sequence read 140 contains all or a portion of a variant in the variant set. See, for example, block 212 of FIG. 2A and the disclosure for the same presented above.

In some embodiments, the using the first plurality of sequence reads to identify support for each variant 144 in a variant set 142 comprises aligning a respective sequence read 140 in the first plurality of sequence reads to a lookup table of variants in order to determine whether the sequence read contains all or a portion of a variant in the variant set. See, for example, block 214 of FIG. 2A and the disclosure for the same presented above.

In some embodiments, the using the first plurality of sequence reads to identify support for each variant 144 in a variant set 142 comprises aligning a sequence read 140 in the first plurality of sequence reads to each entry in a lookup table, where each entry in the lookup table represents a different portion of a genome. See, for example, block 216 of FIG. 2A and the disclosure for the same presented above.

In some embodiments, the subject is a human subject. In some embodiments, the subject is mammalian. In some embodiments the subject is any of the species disclosed above in conjunction with block 204 of FIG. 2.

In some embodiments, the biological sample comprises blood, whole blood, plasma, serum, urine, cerebrospinal fluid, fecal, saliva, sweat, tears, pleural fluid, pericardial fluid, or peritoneal fluid of the subject. That is, the biological sample is a mixture of blood, whole blood, plasma, serum, urine, cerebrospinal fluid, fecal, saliva, sweat, tears, pleural fluid, pericardial fluid, and/or peritoneal fluid of the subject and one or more other components of the subject.

In some embodiments, the biological sample consists of blood, whole blood, plasma, serum, urine, cerebrospinal fluid, fecal, saliva, sweat, tears, pleural fluid, pericardial fluid, or peritoneal fluid of the subject. That is, the biological sample is blood, whole blood, plasma, serum, urine, cerebrospinal fluid, fecal, saliva, sweat, tears, pleural fluid, pericardial fluid, and/or peritoneal fluid of the subject and no other components of the subject.

In some embodiments the classifier makes use of the B score classifier described in U.S. Patent Publication No. 62/642,461, entitled “Method and System for Selecting, Managing, and Analyzing Data of High Dimensionality,” filed 62/642,461, which is hereby incorporated by reference.

In some embodiments, the classifier makes use of the M score classifier described in U.S. Patent Application No. 62/642,480, entitled “Methylation Fragment Anomaly Detection,” filed Mar. 13, 2018, which is hereby incorporated by reference.

In some embodiments, the classifier is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, the classifier is a support vector machine (SVM). SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, N.Y.; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, N.Y.; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In some embodiments, the classifier is a decision tree. Decision trees are described generally by Duda, 2001, Pattern Classification, John Wiley & Sons, Inc., N.Y., pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, N.Y., Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments, the classifier is an unsupervised clustering model. In some embodiments, the classifier is a supervised clustering model. Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering where no preconceived notion of what clusters should form when the training set is clustered are imposed.

In some embodiments, the classifier is a regression model, such as the of the multi-category logit models described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, N.Y.

Alternative method for determining tumor fraction that does not require tumor matching. The methods disclosed above in conjunction with FIG. 2 require the use of a reference set 128 from an aberrant tissue of the subject such as a tumor tissue. Another aspect of the present disclosure provides a method of determining tumor fraction in cell-free nucleic acid of a liquid biological sample of a subject without a requirement for matching allele frequencies to a corresponding tumor sample. This reference free method comprises, at a computer system having one or more processors, and memory storing one or more programs for execution by the one or more processors, obtaining a plurality of sequence reads in electronic form from the liquid biological sample of the subject, where the liquid biological sample comprises cell-free nucleic acid molecules. In some embodiments, any of the methods for obtaining such sequence reads disclosed above in conjunction with blocks 202 through 208 of FIG. 2 are used.

The method further comprises using the plurality of sequence reads to identify support for each variant in a variant set thereby determining an observed frequency of each variant in the first variant set. In some embodiments, any of the methods for using a plurality of sequence reads to identify support for each variant in a variant set, thereby determining an observed frequency of each variant in the variant set, disclosed above in conjunction with blocks 210 through 226 are used.

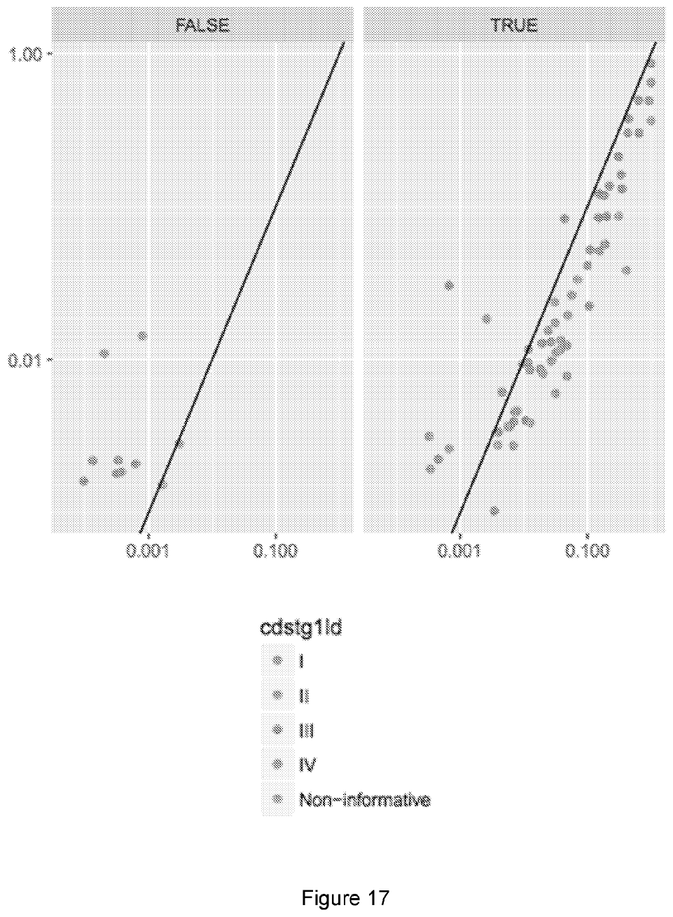

The method further comprises deeming the observed frequency of the variant having the N^{th} highest allele frequency in the variant set to be the tumor fraction in cell-free nucleic acid of the liquid biological sample of the subject, where N is a positive integer other than one (e.g., 1, 2, 3, 4, 5, etc.). FIG. 17 provides a comparison of tumor fraction estimated from tumor variant coverage in cfDNA versus reference free tumor fraction estimates from cfDNA alone. FIG. 17 thus compares the reference free TF estimation from de novo called small variants in cfDNA of the present aspect of the disclosure, with N set to 2 (y-axis) versus TF estimated from assessing tumor mutation coverage in cfDNA using the paired approach described above in conjunction with FIG. 2 (x-axis). To estimate reference free TF, somatic variants were called de novo from ART assay sequencing reads of the CCGA cohort described in Example 12. Variants were filtered after noise modelling, joint modelling with white blood cells (WBC), and edge variant artifact modelling disclosed in U.S. patent application Ser. No. 16/201,912, entitled “Models for Targeted Sequencing,” filed Nov. 27, 2018, which is hereby incorporated by reference. Furthermore, variants underwent variant attribution. See, for example, U.S. patent application Ser. No. 16/201,912, entitled “Models for Targeted Sequencing,” filed Nov. 27, 2018, which is hereby incorporated by reference. Of variants identified as somatic and not attributed to WBC origin, tumor fraction was estimated as the second top ranking variant allele frequency (af_max2). In FIG. 17, results are faceted on whether tumor evidence (at least one tumor mutation read in cfDNA, TRUE) versus no tumor evidence in cfDNA (FALSE). FIG. 17 shows that agreement in the reference free approach of the instant aspect of the present disclosure and the paired approach of FIG. 2 in estimates for samples with positive read evidence down to around a tumor fraction of 1/1000.

In some embodiments, a variant in the variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

In some embodiments, a respective sequence read in the plurality of sequence reads is deemed to support a first variant in the variant set when the respective sequence read contains all or a portion of the first variant, and a respective sequence read in the plurality of sequence reads is deemed to not support the first variant in the variant set when the respective sequence read does not contain the first variant, and a number of sequence reads in the plurality of sequence reads that support the first variant versus a number of sequence reads in the plurality of sequence reads that do not support the first variant determine the observed frequency of the first variant, which estimates the variant frequency of the first variant within the liquid biological sample.

In some embodiments, the subject has a cancer from a single primary site of origin. In some embodiments, the subject has a cancer originating from two or more different organs. In some embodiments, the subject has breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the variant set comprises five or more variants, and each respective variant in the variant set is at a different locus in the genome of the subject. In some embodiments, the variant set consists of between three and twenty variants, and each variant in the variant set is for a different genetic variation in the genome of the subject.

In some embodiments, the variant set consists of between 2 and 200 variants, and each variant in the variant set is for a different genetic variation in the genome of the subject. In some embodiments, the variant set comprises 1000 variants, and each variant in the variant set is for a different genetic variation in the genome of the subject.

In some embodiments, the using the plurality of sequence reads to identify support for each variant in a variant set comprises aligning a sequence read in the plurality of sequence reads to a region in a reference genome in order to determine whether the sequence read contains all or a portion of a first variant.

In some embodiments, the using the plurality of sequence reads to identify support for each variant in a variant set comprises aligning a sequence read in the plurality of sequence reads to a lookup table of variants in order to determine whether the sequence read contains all or a portion of a first variant.

In some embodiments, the using the plurality of sequence reads to identify support for each variant in a variant set comprises aligning a sequence read in the plurality of sequence reads to each entry in a lookup table, wherein each entry in the lookup table represents a different portion of a genome.

In some embodiments, the liquid biological sample comprises or consists of blood, whole blood, plasma, serum, urine, cerebrospinal fluid, fecal, saliva, sweat, tears, pleural fluid, pericardial fluid, or peritoneal fluid of the subject.

In some embodiments, the method further comprises repeating the obtaining a plurality of sequence reads at each respective time point in a plurality of time points across an epoch, from a respective biological sample of the subject taken at each respective time point, where the respective biological sample comprises cell-free nucleic acid molecules, thereby obtaining a corresponding plurality of sequence reads for the subject at each respective time point and determining, for each respective time point in the plurality of time points, support for the variant in the variant set that had the N^{th} highest allele frequency in the original deeming step, thereby determining the state or progression of a disease condition in the subject during the epoch in the form of an increase or decrease of the allele frequency of the variant over the epoch.

In some embodiments, the epoch is a period of months (e.g., between 1 month and 4 months) and each time point in the plurality of time points is a different time point in the period of months. In some embodiments, the epoch is a period of years (e.g., between two and ten years) and each time point in the plurality of time points is a different time point in the period of years. In some embodiments, the epoch is a period of hours (e.g., between one hour and six hours) and each time point in the plurality of time points is a different time point in the period of hours.

In some embodiments, the method further comprises changing a diagnosis of the subject when the allele frequency of the variant is observed to change by a threshold amount (e.g., by ten percent, by twenty percent, by thirty percent relative to a reference amount such as at the time of first measurement) across the epoch.

In some embodiments, the method further comprises changing a prognosis of the subject when the allele frequency of the variant is observed to change by a threshold amount (e.g., by ten percent, by twenty percent, by thirty percent relative to a reference amount such as at the time of first measurement) across the epoch.

In some embodiments, the method further comprises changing a treatment of the subject when the allele frequency of the variant is observed to change by a threshold amount (e.g., by ten percent, by twenty percent, by thirty percent relative to a reference amount such as at the time of first measurement) across the epoch.

In some embodiments, the disease condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof). In some embodiments, the disease condition is a stage of cancer (e.g., a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer). In some embodiments, the disease condition is a predetermined subtype of a cancer.

In some embodiments, the method further comprises applying the plurality of sequence reads to a trained classifier thereby obtaining a classifier result, where the trained classifier result indicates whether the subject has a first cancer condition, and using the trained classifier result as a basis for diagnosis of the subject for the first cancer condition when the tumor fraction is between 0.003 and 1.0 and the trained classifier result indicates that the subject has the first cancer condition. In some such embodiments, the first cancer condition is a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof). In some such embodiments, the first cancer condition is a subtype of a cancer (e.g., a subtype of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer). In some such embodiments, the first tumor fraction is between 0.003 and 1.0 and the first cancer condition is a tissue of origin of a cancer. In some embodiments, the trained classifier is a neural network, a support vector machine, a decision tree, an unsupervised clustering model, a supervised clustering model, or a regression model.

### EXAMPLE 1

Increase in Median ctDNA Fraction by Cancer by Stage

Referring to FIG. 4, subjects are grouped by cancer stages I, II, III, and IV, regardless of the type of cancer that they have. In FIG. 4, the x-axis indicates which cancer stage each subject has and while the y-axis indicates the observed ctDNA fraction for each subject. The method used to compute the cfDNA fraction for each subject comprises obtaining a first plurality of sequence reads 140 in electronic form from a biological sample of each subject in a cohort, where the biological sample comprises cell-free nucleic acid molecules. The first plurality of sequence reads 140 are used to identify support for each variant 144 in a variant set 142 for the biological sample thereby determining an observed frequency (support 146) of each variant 144 in the variant set 142. The observed frequency (support 146) of each respective variant 144 in the variant set 142 is compared to a corresponding reference frequency 132 for the respective variant in a reference set 128. Each such corresponding reference frequency 132 in the reference set 128 is a frequency of a respective variant in a first aberrant tissue sample obtained from the subject. Subjects that do not have positive reads, meaning that subjects that do not have sequence reads 140 that support the variants observed in the matched reference set of such subjects, are not included in FIG. 4.

In the case where the variant set consists of a single variant, the comparison of the observed frequency of each respective variant 144 in the variant set 142 to a corresponding reference frequency 132 for the respective variant in a reference set 128 comprise taking a ratio of the frequency of the variant in the variant set (obtained from sequence reads of cfDNA of the biological sample) to the frequency of the same variant (obtained from sequence reads of DNA in the aberrant tissue) in the reference set.

In the case where the variant set consists of two or more variants, the comparison of the observed frequency of each respective variant 144 in the variant set 142 to a corresponding reference frequency 132 for the respective variant in a reference set 128 comprises taking a ratio of the frequency of each respective variant in the variant set (obtained from sequence reads of cfDNA of the biological sample) to the frequency of the same variant as the respective variant (obtained from sequence reads of DNA in the aberrant tissue) in the reference set. That is, there is a one-to-one correspondence between variants in the reference set and variants in the variant set.

FIG. 4 thus provides an analysis of how ctDNA fraction varies by cancer stage regardless of cancer type, among subjects that have cell free sequence reads that support their underlying cancer. FIG. 4 thus shows that, as the disease is more severe as determined by clinically staging (stages 1 through 4), more evidence of tumor fraction (larger ctDNA fraction) is found in the cfDNA. While FIG. 4 shows that while this is the general case across the CCGA cohort (see Example 12 for details of the CCGA cohort), there are violations (outliers) to this trend. Such outliers in FIG. 4 are suggestive and best explained by clinical misclassification. FIG. 4 thus shows a fundamental component of the underlying disease, which is the generally expected tumor fraction rates in the cfDNA. FIG. 4 also shows that stage 4 has some individuals that have very low shedding rates indicating that there are different sub-states within stage 4.

FIG. 4 illustrates that shedding rates (ctDNA fraction) can be used as a basis for establishing meaningful and informative thresholds, from observed frequencies of the variants in the reference set. That is, for example, given observed frequencies of variants in the aberrant tissue of a given subject, and optionally information regarding expected ctDNA fraction for subjects having a particular phase of cancer, a threshold for the given cancer subject can be determined and evaluated against the observed frequency of the variants in a variant set for the given subject in order to classify the subject as having or not having the condition (e.g., a clinical stage of a given cancer). For example, referring to FIG. 4, a threshold of 0.05 may be used to analyze whether a subject has stage I of a given cancer. In this example, an aberrant tissue, such as a tumor, is obtained from a patient and used to determine a reference frequency for each respective variant in a first reference set. In fact, in some embodiments, the frequency of various possible variants is used to define the variants of the reference set. Next, cell free nucleic acid is obtained from a biological sample, other than the aberrant tissue, of the same subject and the variant frequency of the same variants that are in the reference set are determined from sequence reads of the cell free nucleic acids in the biological sample, thereby forming the observed ctDNA frequency of each respective variant in the first variant set. A comparison of the ctDNA frequency to the reference frequencies to determine if the threshold condition of 0.05 is satisfied then provides a basis for determining whether or not the subject has stage I cancer or not. For instance, if the comparison indicates that the ctDNA fraction is more than 0.05, this indicates that the subject has a more advanced stage of cancer. On the other hand, observation of a ctDNA fraction, formed from the observed frequency of each respective variant in the first variant set that is less than 0.001, is consistent with a finding that the subject has stage I of a given cancer.

### EXAMPLE 2

Ability to Detect ctDNA as a Function of Stage of Breast Cancer

In FIG. 5, each point is the ctDNA fraction of an individual subject that has breast cancer in the CCGA cohort described in Example 12 below in which WGS sequencing was used. The method used to compute the cfDNA fraction for each subject comprises obtaining a first plurality of sequence reads 140 in electronic form from a biological sample of each subject in a cohort, where the biological sample comprises cell-free nucleic acid molecules. The first plurality of sequence reads 140 are used to identify support for each variant 144 in a variant set 142 for the biological sample thereby determining an observed frequency (support 146) of each variant 144 in the variant set 142. The observed frequency (support 146) of each respective variant 144 in the variant set 142 is compared to a corresponding reference frequency 132 for the respective variant in a reference set 128. Each such corresponding reference frequency 132 in the reference set 128 is a frequency of a respective variant in a first aberrant tissue sample obtained from the subject.

In addition to plotting the ctDNA fraction of each subject, FIG. 5 breaks the subjects out by stage of breast cancer and annotates each subject by one of three different classes. The first class (red triangles) is the case where the sequence reads 140 of a biological sample of the subject provide sufficient basis to independently call at least one variant 144 that matches one of the variants in the reference set. Thus, in such embodiments, the aberrant tissue sample (e.g., tumor) did not make use of the cell free DNA variants and vice versa and yet the targeted assay based upon the sequence reads from the biological sample (e.g., blood) independently identifies the variant without relying on sequencing data from the tumor. The second class (blue triangles) represent read evidence based analysis for a tumor variant where cfDNA is observed to have sequence reads that support at least one variant called by direct tumor sequencing of the tumor. The third class, (black circles) indicates that there is no evidence that the cfDNA sequence reads have variants that match the variants directly observed in the aberrant tissue (breast cancer tumors).

FIG. 5 indicates a very large dynamic range for tumor fraction that is observed within each tumor stage. FIG. 5 further indicates that when the tumor fraction is one percent or above, the assay detects the breast cancer with an appreciable confidence interval. Between 1.0 percent and 0.1 percent the performance of the assay decreases. For the black points, the confidence intervals go all the way to zero, meaning that for such individuals one can be confident that these individual samples do not exceed the tumor fraction. Thus, analyzing stage II in FIG. 5, one can see that a substantial population of the subjects with stage II breast cancer have a tumor fraction that is below the limits of the assay detection. In other words, that there are numerous subjects with stage II breast cancer that have low shedding rates, indicating that the identification of ctDNA in the cfDNA for such subjects falls below the limits of detection.

### EXAMPLE 3

Ability to Detect Cancer as a Function of cfDNA Fraction

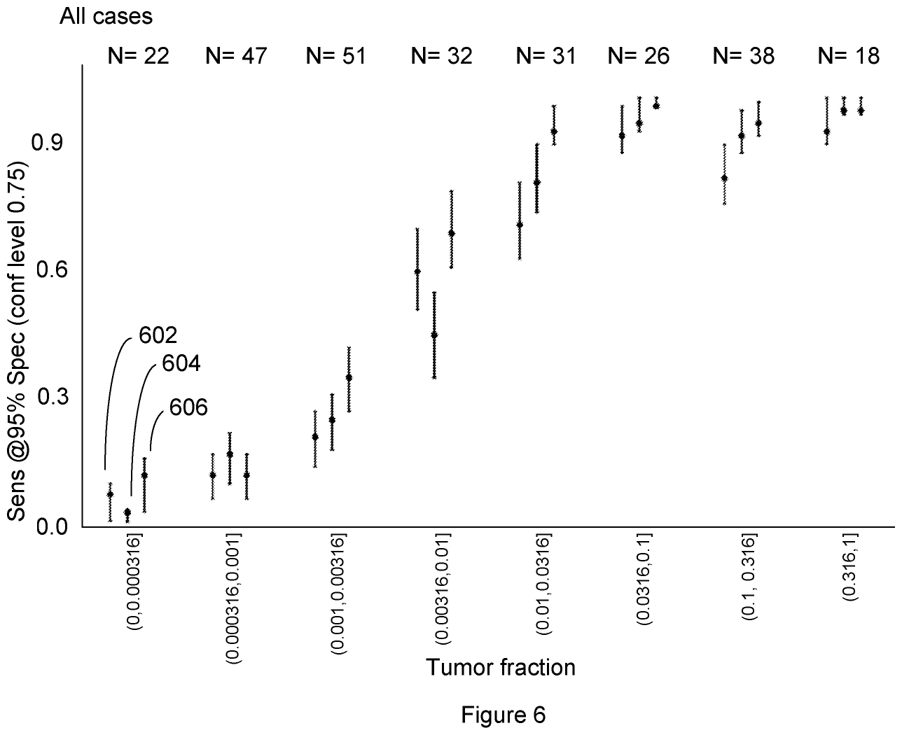

FIG. 6 provides an estimate of how many individuals were classified as having cancer, using one of three different classifiers (Y-axis) as a function of the cfDNA fraction (X-axis), in the CCGA cohort described in Example 12 below in which WGS sequencing was used. That is, subjects are grouped into one of eight bins on the X-axis based on cfDNA fraction and then the mean and range of the sensitivity for each such bin of subjects at 95% specificity is plotted on the Y-axis for each of three different classifiers. For each cfDNA bin in FIG. 6, the three different classifiers are, from left to right (and using the bin (0,0.000316] to illustrate), “A score” 602, “B score” 604, and “M score” 606.

The A score classifier, described herein is a classifier of tumor mutational burden based on targeted sequencing analysis of nonsynonymous mutations. For example, a classification score (e.g., “A score”) can be computed using logistic regression on tumor mutational burden data, where an estimate of tumor mutational burden for each individual is obtained from the targeted cfDNA assay. In some embodiments, a tumor mutational burden can be estimated as the total number of variants per individual that are: called as candidate variants in the cfDNA, passed noise-modeling and joint-calling, and/or found as nonsynonymous in any gene annotation overlapping the variants. The tumor mutational burden numbers of a training set can be fed into a penalized logistic regression classifier to determine cutoffs at which 95% specificity is achieved using cross-validation. An example of the cross-validated performance is shown in FIG. 6. Additional details on A score can be found, for example, in R. Chaudhary et al., 2017, “Estimating tumor mutation burden using next-generation sequencing assay,” Journal of Clinical Oncology, 35(5), suppl.e14529, pre-print online publication, which is hereby incorporated by reference herein in its entirety.

The B score classifier is described in U.S. Patent Publication No. 62/642,461, entitled “Method and System for Selecting, Managing, and Analyzing Data of High Dimensionality,” filed 62/642,461, which is hereby incorporated by reference. In accordance with the B score method, a first set of sequence reads of nucleic acid samples from healthy subjects in a reference group of healthy subjects are analyzed for regions of low variability. Accordingly, each sequence read in the first set of sequence reads of nucleic acid samples from each healthy subject are aligned to a region in the reference genome. From this, a training set of sequence reads from sequence reads of nucleic acid samples from subjects in a training group are selected. Each sequence read in the training set aligns to a region in the regions of low variability in the reference genome identified from the reference set. The training set includes sequence reads of nucleic acid samples from healthy subjects as well as sequence reads of nucleic acid samples from diseased subjects who are known to have the cancer. The nucleic acid samples from the training group are of a type that is the same as or similar to that of the nucleic acid samples from the reference group of healthy subjects. From this it is determined, using quantities derived from sequence reads of the training set, one or more parameters that reflect differences between sequence reads of nucleic acid samples from the healthy subjects and sequence reads of nucleic acid samples from the diseased subjects within the training group. Then, a test set of sequence reads associated with nucleic acid samples comprising cfNA fragments from a test subject whose status with respect to the cancer is unknown is received, and the likelihood of the test subject having the cancer is determined based on the one or more parameters.

The M score classifier is described in U.S. Patent Application No. 62/642,480, entitled “Methylation Fragment Anomaly Detection,” filed Mar. 13, 2018, which is hereby incorporated by reference.

FIG. 6 indicates that above a cfDNA fraction of three percent, all three classifiers detect the individuals that have the cancer. For lower cfDNA fractions, the M score classifier has statistically significant improvement in sensitivity relative to the B score classifier in the interval of (0.00316,0.01]. Thus, for the intermediate shedding rates, the M score classifier appears to be superior. For lower shedding rates, cfDNA fractions less than 00.316, none of the classifier appear to be suitable. FIG. 6 thus motivates how to refine the cancer detection classifier moving forward. On the X-axis, the comma between two values means range, round bracket means exclusive of, and square bracket means “inclusive of” For cfDNA fractions of three percent or greater the classifiers each have a sensitivity rate of 95 percent or greater with a false positive rate of five percent.

### EXAMPLE 4

Ability to Call Breast Cancer as a Function of cfDNA Fraction, Sequencing Protocol, and Breast Cancer Subtype

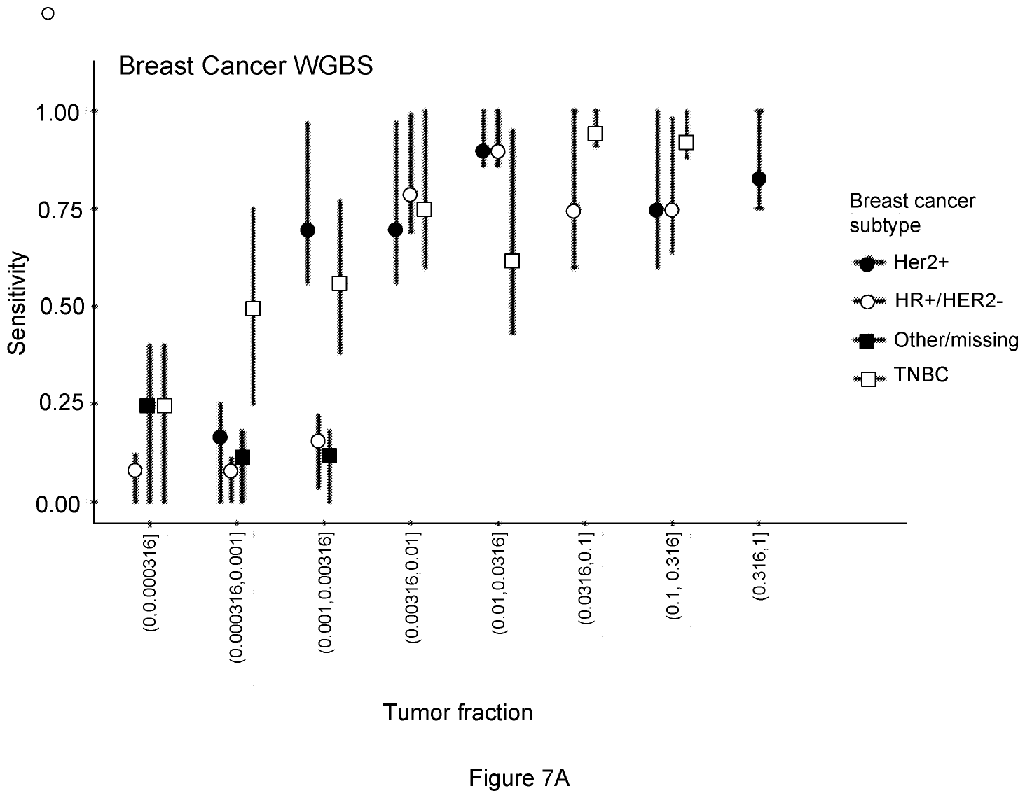

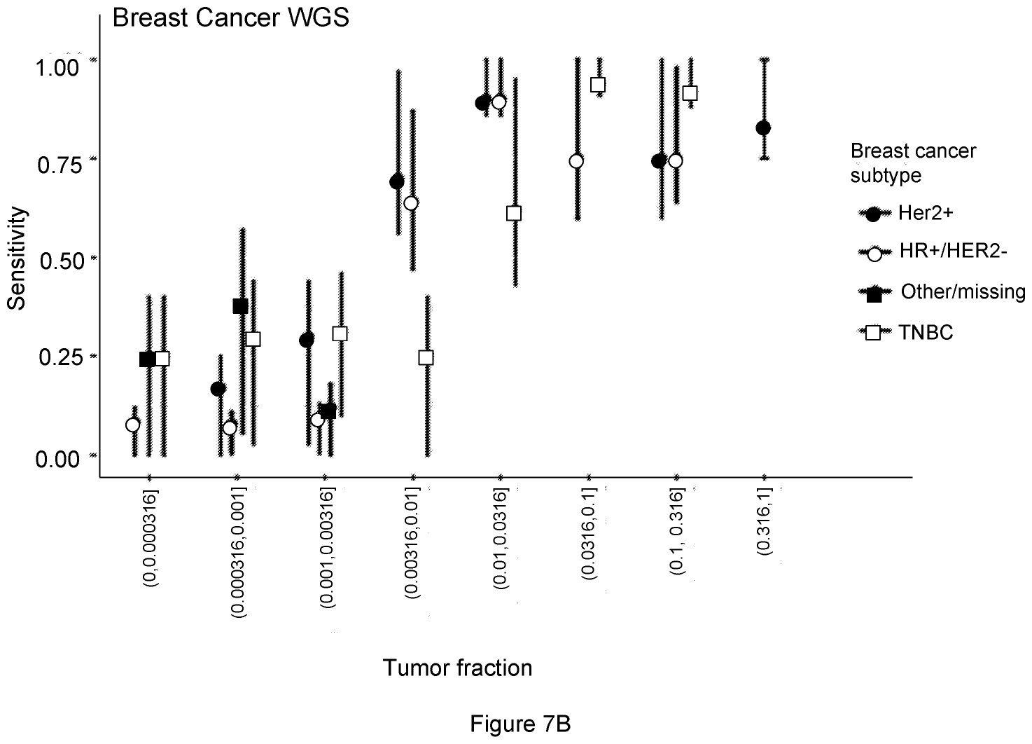

FIGS. 7A and 7B detail the sensitivity of a breast cancer calling classifier using whole-genome bisulfite sequencing (WGBS) (FIG. 7A) and whole genome sequencing (WGS) (FIG. 7B) to perform variant calling, and thus calling of subjects as having or not having breast cancer, as a function of cfDNA fraction for four different subtypes of breast cancer, HER2+ (solid circles), HR+/HER2− (hollow circles), other/missing (solid squares) and TNBC (hollow squares) using the CCGA cohort described in Example 12 below. FIG. 7 demonstrates that, given a breast cancer subtype (e.g., HER2+ versus Hormone Receptor+ (HR+), there are differences in classifier sensitivity for different types of variant calling methodologies. FIG. 7 further indicates that the signal availability for the more aggressive cancer for HER2+ is much better than the less aggressive forms of breast cancer. See, for example, the sensitivity of the (0.001,0.00316] interval in FIG. 7A. In FIG. 7, sensitivity is a cancer versus non-cancer assignment. For FIG. 7, the calling of subjects as “having cancer” and “not having breast cancer” based on WGBS and WGS data, respectively, do not make use of any ctDNA shedding information. FIG. 7 demonstrates that cancer detection classifier work better for those cancers that have higher ctDNA fractions.

### EXAMPLE 5

Precision of a Whole-Genome Bisulfite Sequencing Multi-class Cancer Type Classifier as a Function of cfDNA Fraction

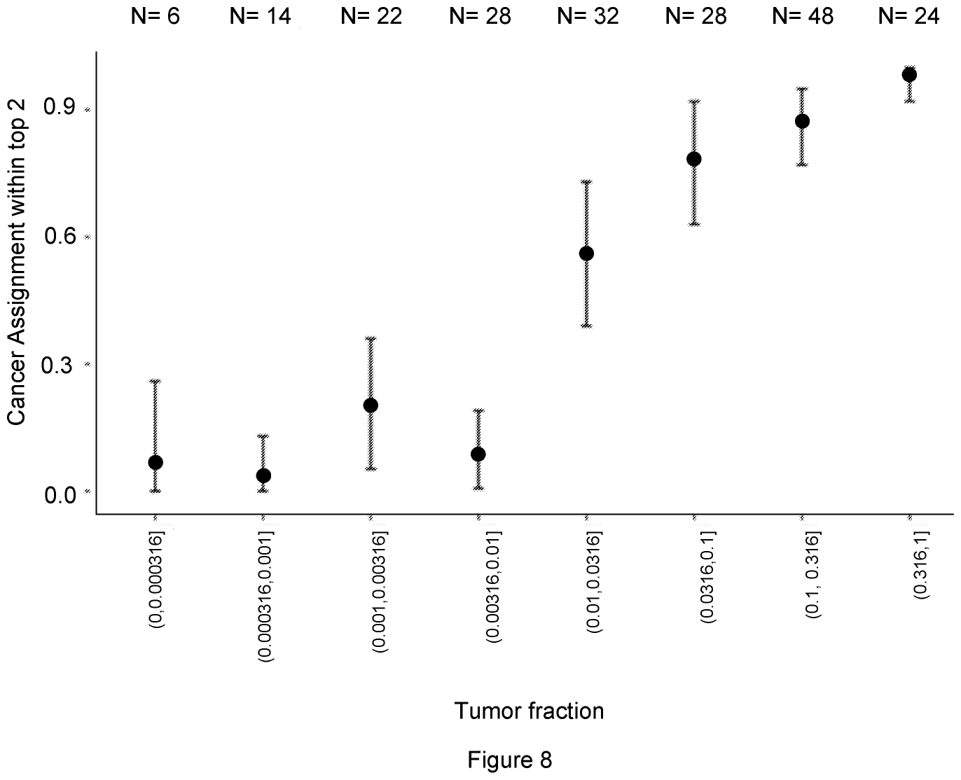

FIG. 8 details the precision of a multi-class classifier for the CCGA cohort of subjects (Example 12 below) that have been sequenced using whole genome bisulfite sequencing (WGBS) spanning the spectrum of different cancers identified in FIG. 3 as a function of ctDNA fraction. For details regarding WGBS, see, for example, Example 13. See also, U.S. patent application Ser. No. 62/642,480, entitled “Methylation Fragment Anomaly Detection,” filed Mar. 13, 2018, which is hereby incorporated by reference. As illustrated in FIG. 8, the cohort is binned into eight different cfDNA fraction bins and the precision, defined as the ability to place the correct cancer for a given subject into the top two cancer class probabilities, of the WGBS classifier for each such bin, and the number of subjects in the cohort in each such bin is provided. FIG. 8 suggests that a threshold ctDNA fraction level is needed in order to achieve the correct assignment using the WGBS multi-class cancer type classifier.

### EXAMPLE 6

Percentage of Subjects that Exhibit a Minimum ctDNA Fraction as a Function of Clinical Stage

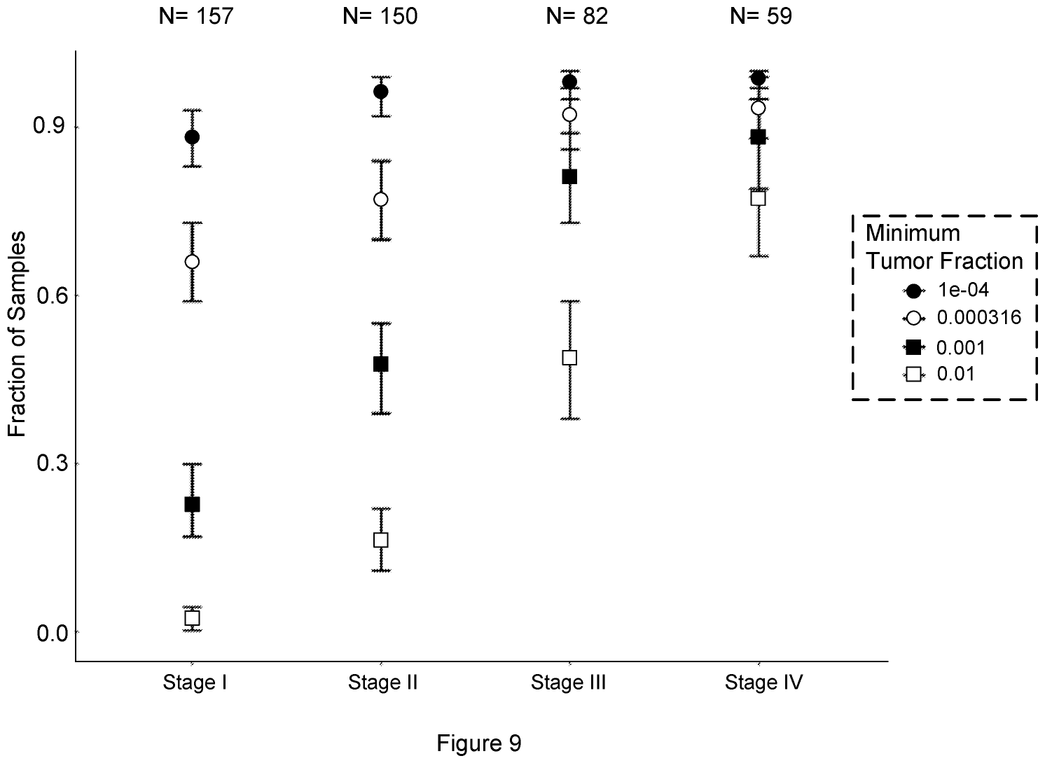

FIG. 9 illustrates the number of samples in the CCGA cohort that exhibit a minimum ctDNA fraction across all cancers represented by the cohort. As was the case in Example 1, the method used to compute the cfDNA fraction for each subject disclosed in FIG. 9 comprises obtaining a first plurality of sequence reads 140 in electronic form from a biological sample of each subject in the cohort, where the biological sample comprises cell-free nucleic acid molecules. The first plurality of sequence reads 140 are used to identify support for each variant 144 in a variant set 142 for the biological sample thereby determining an observed frequency (support 146) of each variant 144 in the variant set 142. The observed frequency (support 146) of each respective variant 144 in the variant set 142 is compared to a corresponding reference frequency 132 for the respective variant in a reference set 128 in order to determine the ctDNA fraction in each subject. Each such corresponding reference frequency 132 in the reference set 128 is a frequency of a respective variant in a first aberrant tissue sample obtained from the subject.

FIG. 9 discloses that the percentage of subjects in the cohort that exhibit a ctDNA fraction of 0.01 climbs from just above 0.00 for all stage I cancers represented by the cohort (N=157 subjects in the cohort with Stage I cancers) to about 0.75 for all stage II cancers represented by the cohort (N=59 subjects in the cohort with Stage IV cancers). FIG. 9 illustrates the available information in the ctDNA tumor fraction of cancer patients that can be used in order to classify the condition of subjects in accordance with the present disclosure, including the methods described in FIG. 2. Examples 1 through 6 collectively show that the methods of the present disclosure are able to classify subjects, evaluate the performance of classifiers based ctDNA fraction, and evaluate the quality of signal given a fixed ctDNA fraction across different cancer types. Advantageously, examples 1 through 6 collectively show that the disclosed systems and methods are able to detect more aggressive forms of cancers, which is highly desirable.

### EXAMPLE 7

Computer Models that Combine ctDNA Fraction with Information Derived from Digital Pathology of Tumors

Examples 1 through 6 indicate that the ctDNA fraction determined in accordance with the methods of the present disclosure may be combined with information obtained from digital pathology to feed a model that predicts the aggressiveness of a given cancer. As such, the present disclosure demonstrates the utility of models that takes into account ctDNA fraction and that further includes digital pathology in order to determine the aggressiveness of a given cancer condition of a particular subject. In such embodiments, the cfDNA fraction for a subject is determined by obtaining a first plurality of sequence reads 140 in electronic form from a biological sample of the subject, where the biological sample comprises cell-free nucleic acid molecules (e.g., from the blood of the subject). The first plurality of sequence reads 140 are used to identify support for each variant 144 in a variant set 142 for the biological sample thereby determining an observed frequency (support 146) of each variant 144 in the variant set 142 in accordance with the teachings of the present disclosure. The observed frequency (support 146) of each respective variant 144 in the variant set 142 is compared to a corresponding reference frequency 132 for the respective variant in a reference set 128 in order to determine the ctDNA fraction of the subject. Such reference frequencies 132 are obtained from sequence reads taken from a tumor or a tumor fraction of the subject. Moreover, one or more sections of the tumor or tumor fraction are analyzed using computer vision techniques to estimate density, how many immune cells are infiltrating, estimate necrosis, and/or estimate rate of proliferation, or other parameters associated with aggressiveness of a cancer. This information is then combined with the ctDNA as input to a classifier that evaluates the aggressiveness of the cancer of the subject and/or any other state associated with the cancer.

### EXAMPLE 8

Positive Association of ctDNA Fraction with Tumor Size

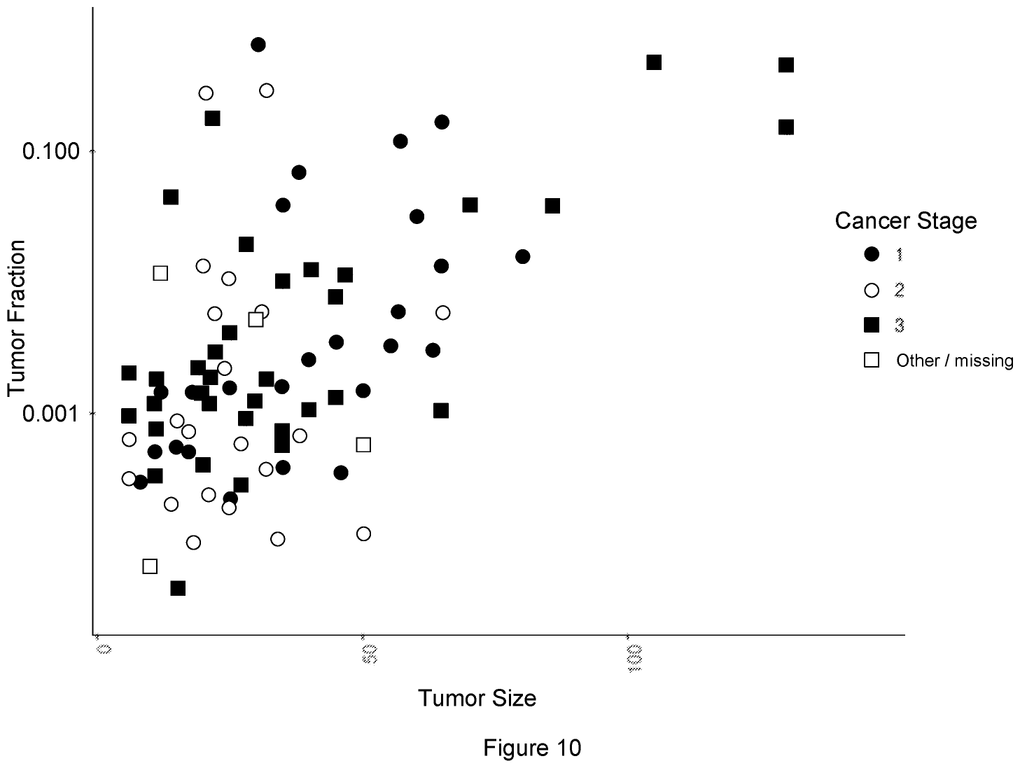

FIG. 10 illustrates the positive association of tumor size with ctDNA fraction, across all stages of cancer using the CCGA cohort described in Example 12. Since tumor size is positively associated with cancer aggressiveness in many instances, Example 8 provides additional support for the use of cfDNA fraction to classify subjects in accordance with the present disclosure, including the methods disclosed in conjunction with FIG. 2, the additional embodiments disclosed below, and the claims of the present disclosure.

### EXAMPLE 9

Association of ctDNA Fraction to Ki67 Marker for Proliferation

Ki-67 is a nuclear protein associated with cellular proliferation. See, Gerdes et al., 1983, “Production of a mouse monoclonal antibody reactive with human nuclear antigen associated with cell proliferation,” Int. J. Cancer 31(1), 13-20, which is hereby incorporated by reference. One method for analyzing the Ki-67 antigen in a subject is the immunohistochemical evaluation. It has been shown that the Ki-67 nuclear antigen is expressed in certain phases of the cell cycle namely S, G1, G2, and M phases, but is nonexisting in G0. See, for example, Gerdes et al., 1984, “Cell cycle analysis of a cell proliferation-associated human nuclear antigen defined by the monoclonal antibody Ki-67,” J Immunol. 133(4), 1710-1715; and Scholzen and Gerdes, 2000, “The Ki-67 protein: from the known and the unknown,” J Cell Physiol. 182(3), 311-322, each of which is hereby incorporated by reference. In samples from normal breast tissue, it has been found that Ki-67 is also expressed at low levels (<3% of cells) in ER-negative cells, but not in ER-positive cells. See, for example, Urruticoechea et al., 2005, “Proliferation marker Ki-67 in early breast cancer, J Clin Oncol. 23: 7212-7220, which is hereby incorporated by reference. By means of immunostaining with the monoclonal antibody Ki-67, it is possible to assess the growth fraction of neoplastic cell populations.

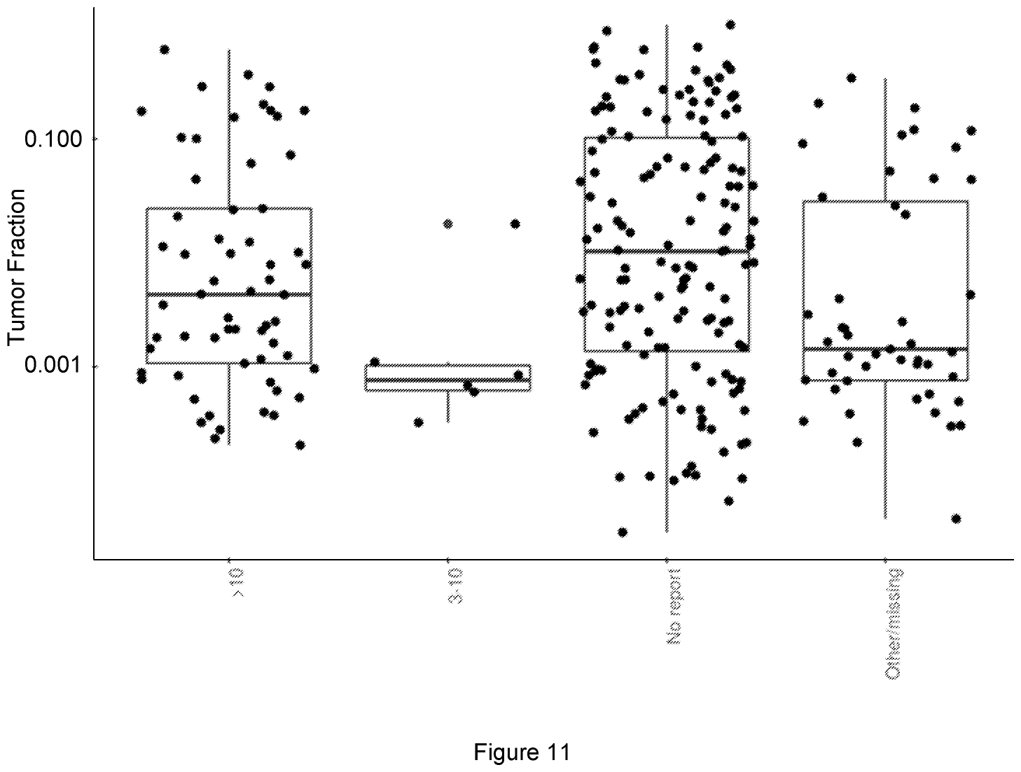

For this example, immunohistochemical staining is conducted and the proportion of the malignant cells staining positive for the nuclear antigen Ki-67 is evaluated in a quantitative and visual way using light microscopes. Ki-67 values are acquired as the percentage of positively marking malignant cells using the anti-human Ki-67 monoclonal antibody. In FIG. 11, the Ki-67 percentage score is defined as the percentage of positively stained tumor cells among the total number of malignant cells assessed. See, Inwald, 2013, “Ki-67 is a prognostic parameter in breast cancer patients: results of a large population-based cohort of the cancer registry,” Breast Cancer Res. Treat. 139(2): 539-552, which is hereby incorporated by reference.

In FIG. 11, the cfDNA fraction for each given subject in the CCGA cohort described in Example 12 below exhibiting a solid invasive cancer is determined by obtaining a first plurality of sequence reads 140 in electronic form from a biological sample of the subject, where the biological sample comprises cell-free nucleic acid molecules (e.g., from the blood of the subject). The first plurality of sequence reads 140 are used to identify support for each variant 144 in a variant set 142 for the biological sample thereby determining an observed frequency (support 146) of each variant 144 in the variant set 142. The observed frequency (support 146) of each respective variant 144 in the variant set 142 is compared to a corresponding reference frequency 132 for the respective variant in a reference set 128 in order to determine the ctDNA fraction of the subject. Such reference frequencies 132 are obtained from sequence reads taken from the tumor or a tumor fraction of the subject from which the Ki-67 values were obtained.

In FIG. 11, in the far left column, samples that have a Ki-67 score greater than 10, shows that there are a number of samples in the tail that have shedding rate greater than 0.1000. This suggests that Ki-67, in conjunction with ctDNA fraction may provide a basis for diagnosing a condition of a subject, such as a more aggressive form of breast cancer.

### EXAMPLE 10

Obtaining a Plurality of Sequence reads

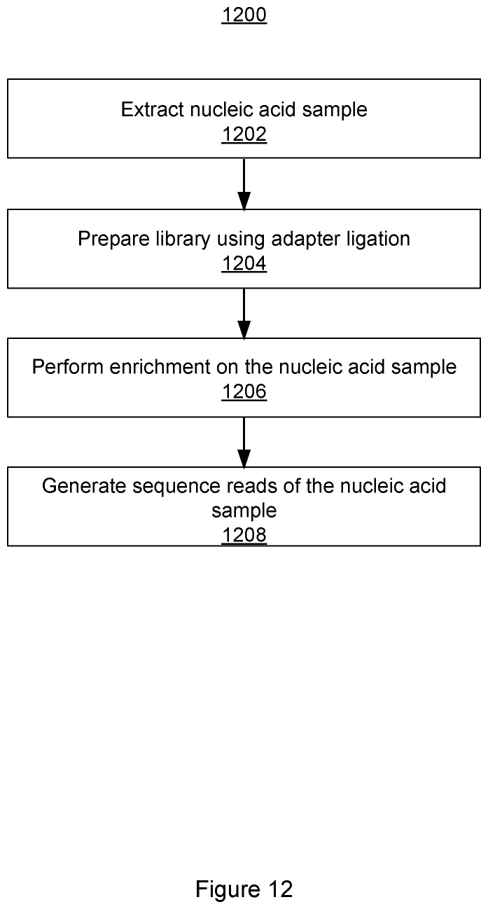

FIG. 12 is a flowchart of a method 1200 for preparing a nucleic acid sample for sequencing according to one embodiment. The method 1200 includes, but is not limited to, the following steps. For example, any step of the method 1200 may comprise a quantitation sub-step for quality control or other laboratory assay procedures known to one skilled in the art.

In block 1202, a nucleic acid sample (DNA or RNA) is extracted from a subject. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In block 1204, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment. This provides a way to identify sequence reads that came from the same original fragment in downstream analysis.

In block 1206, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes may cover overlapping portions of a target region.

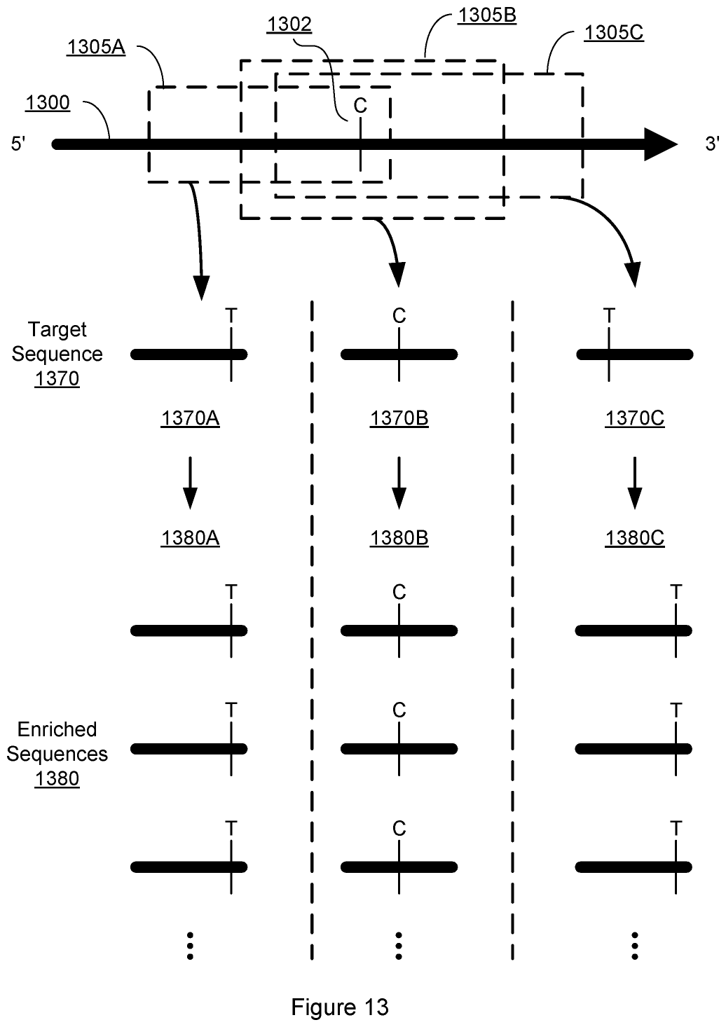

FIG. 13 is a graphical representation of the process for obtaining sequence reads according to one embodiment. FIG. 13 depicts one example of a nucleic acid segment 1300 from the sample. Here, the nucleic acid segment 1300 can be a single-stranded nucleic acid segment, such as a single stranded. In some embodiments, the nucleic acid segment 1300 is a double-stranded cfDNA segment. The illustrated example depicts three regions 1305A, 1305B, and 1305C of the nucleic acid segment 160 that can be targeted by different probes. Specifically, each of the three regions 165A, 165B, and 165C includes an overlapping position on the nucleic acid segment 160. An example overlapping position is depicted in FIG. 13 as the cytosine (“C”) nucleotide base 1302. The cytosine nucleotide base 1302 is located near a first edge of region 1305A, at the center of region 1305B, and near a second edge of region 1305C.

In some embodiments, one or more (or all) of the probes are designed based on a gene panel to analyze particular mutations or target regions of the genome (e.g., of the human or another organism) that are suspected to correspond to certain cancers or other types of diseases. By using a targeted gene panel rather than sequencing all expressed genes of a genome, also known as “whole exome sequencing,” the method 1200 may be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 1300 using one or more probes results in an understanding of a target sequence 1370. As shown in FIG. 13, the target sequence 1370 is the nucleotide base sequence of the region 1305 that is targeted by a hybridization probe. The target sequence 1370 can also be referred to as a hybridized nucleic acid fragment. For example, target sequence 1370A corresponds to region 1305A targeted by a first hybridization probe, target sequence 1370B corresponds to region 1305B targeted by a second hybridization probe, and target sequence 1370C corresponds to region 1305C targeted by a third hybridization probe. Given that the cytosine nucleotide base 1302 is located at different locations within each region 1305A-C targeted by a hybridization probe, each target sequence 1370 includes a nucleotide base that corresponds to the cytosine nucleotide base 1302 at a particular location on the target sequence 1370.

After a hybridization step, the hybridized nucleic acid fragments are captured and may also be amplified using PCR. For example, the target sequences 1370 can be enriched to obtain enriched sequences 1380 that can be subsequently sequenced. In some embodiments, each enriched sequence 1380 is replicated from a target sequence 1370. Enriched sequences 1380A and 1380C that are amplified from target sequences 1370A and 1370C, respectively, also include the thymine nucleotide base located near the edge of each sequence read 180A or 180C. As used hereafter, the mutated nucleotide base (e.g., thymine nucleotide base) in the enriched sequence 1380 that is mutated in relation to the reference allele (e.g., cytosine nucleotide base 1302) is considered as the alternative allele. Additionally, each enriched sequence 1380B amplified from target sequence 1370B includes the cytosine nucleotide base located near or at the center of each enriched sequence 1380B.

In block 1208, sequence reads are generated from the enriched DNA sequences, e.g., enriched sequences 180 shown in FIG. 13. Sequencing data may be acquired from the enriched DNA sequences by known means in the art. For example, the method 1200 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads are aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. In one example, the first read R₁ is sequenced from a first end of a nucleic acid fragment whereas the second read R₂ is sequenced from the second end of the nucleic acid fragment. Therefore, nucleotide base pairs of the first read R₁ and second read R₂ are aligned consistently (e.g., in opposite orientations) with nucleotide bases of the reference genome in the example. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as variant calling described above in conjunction with FIG. 2 as well as in Example 11.

### EXAMPLE 11

Identifying Variants

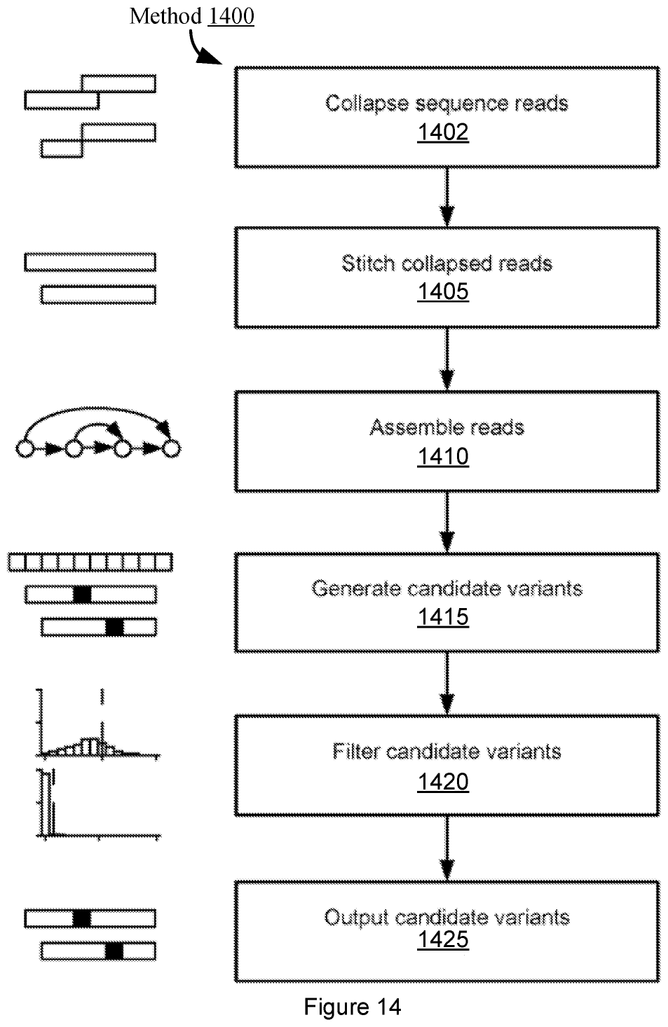

FIG. 14 is flowchart of a method 1400 for determining variants of sequence reads according to one embodiment. In some embodiments, variant calling (e.g., for SNVs and/or indels) based on input sequencing data is performed as discussed above in conjunction with FIG. 2 and Example 10. At step 1402, aligned sequence reads of the input sequencing data are collapsed. In one embodiment, collapsing sequence reads includes using UMIs, and optionally alignment position information from sequencing data of an output file (e.g., from the method described in Example 10) to collapse multiple sequence reads into a consensus sequence for determining the most likely sequence of a nucleic acid fragment or a portion thereof. In some embodiments, the unique sequence tag is from about 4 to 20 nucleic acids in length. Since the UMIs are replicated with the ligated nucleic acid fragments through enrichment and PCR, a determination can be made that certain sequence reads originated from the same molecule in a nucleic acid sample. In some embodiments, sequence reads that have the same or similar alignment position information (e.g., beginning and end positions within a threshold offset) and include a common UMI are collapsed, a collapsed read is generated (also referred to herein as a consensus read) to represent the nucleic acid fragment. A consensus read is designated as “duplex” if the corresponding pair of collapsed reads have a common UMI, indicating that both positive and negative strands of the originating nucleic acid molecule is captured; otherwise, the collapsed read is designated “non-duplex.” In some embodiments, other types of error correction are performed on sequence reads as an alternate to, or in addition to, collapsing sequence reads.

At step 1405, the collapsed reads are stitched based on the corresponding alignment position information. In some embodiments, alignment position information between a first read and a second read is compared to determine whether nucleotide base pairs of the first and second reads overlap in the reference genome. In one use case, responsive to determining that an overlap (e.g., of a given number of nucleotide bases) between the first and second reads is greater than a threshold length (e.g., threshold number of nucleotide bases), the first and second reads are designated as “stitched”; otherwise, the collapsed reads are designated “unstitched.” In some embodiments, a first and second read are stitched if the overlap is greater than the threshold length and if the overlap is not a sliding overlap. For example, a sliding overlap may include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 1410, reads are assembled into paths. In some embodiments, this involves assembling reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). Collapsed reads are aligned to a directed graph such that any of the collapsed reads may be represented in order by a subset of the edges and corresponding vertices.

In some embodiments, sets of parameters describing directed graphs and processes directed graphs are determined. The set of parameters may include a count of successfully aligned k-mers from collapsed reads to a k-mer represented by a node or edge in the directed graph. The directed graphs and corresponding sets of parameters are stored in some embodiments for later retrieval to update graphs or generate new graphs. For instance, a compressed version of a directed graph (e.g., or modify an existing graph) based on the set of parameters may be generated. In one use case, in order to filter out data of a directed graph having lower levels of importance, nodes or edges having a count less than a threshold value are removed (e.g., “trimmed” or “pruned”), while nodes or edges having counts greater than or equal to the threshold value are maintained.

At step 1415, the variant caller 240 generates candidate variants from the assembled paths. In one embodiment, candidate variants are generated by comparing a directed graph (which may have been compressed by pruning edges or nodes in step 1410) to a reference sequence of a target region of a genome. Edges of the directed graph may be aligned to the reference sequence, and the genomic positions of mismatched edges and mismatched nucleotide bases adjacent to the edges recorded as the locations of candidate variants. In some embodiments, the genomic positions of mismatched edges and mismatched nucleotide bases to the left and right of edges are recorded as the locations of called variants. Additionally, candidate variants may be generated based on the sequencing depth of a target region. In particular, there may be more confidence in identifying variants in target regions that have greater sequencing depth, for example, because a greater number of sequence reads help to resolve (e.g., using redundancies) mismatches or other base pair variations between sequences.

In one embodiment, candidate variants are generated using a model to determine expected noise rates for sequence reads from a subject. The model may be a Bayesian hierarchical model, though in some embodiments, one or more different types of models are used. Moreover, a Bayesian hierarchical model may be one of many possible model architectures that may be used to generate candidate variants and which are related to each other in that they all model position-specific noise information in order to improve the sensitivity/specificity of variant calling. More specifically, the model may be trained using samples from healthy individuals to model the expected noise rates per position of sequence reads.

Further, multiple different models may be used for application post-training. In one example, a first model is trained to model SNV noise rates and a second model is trained to model indel noise rates. Further, parameters of the model may be used to determine a likelihood of one or more true positives in a sequence read. A quality score (e.g., on a logarithmic scale) based on the likelihood can be determined. In one example, the quality score is a Phred quality score Q=−10 log₁₀ P, where P is the likelihood of an incorrect candidate variant call (e.g., a false positive). Other models such as a joint model may use output of one or more Bayesian hierarchical models to determine expected noise of nucleotide mutations in sequence reads of different samples.

At step 1420, the candidate variants are filtered using one or more types of models or filters. In one example, the candidate variants are scored using a joint model, edge variant prediction model, or corresponding likelihoods of true positives or quality scores. In addition, edge variants and/or non-synonymous mutations may be filtered using an edge filter and/or nonsynonymous filter, respectively.

At step 1425, the filtered candidate variants are outputted. In some embodiments, some or all of the determined candidate variants are outputted along with corresponding one scores from the filtering steps.

### EXAMPLE 12

Cell-Free Genome Atlas Study (CCGA) Cohort

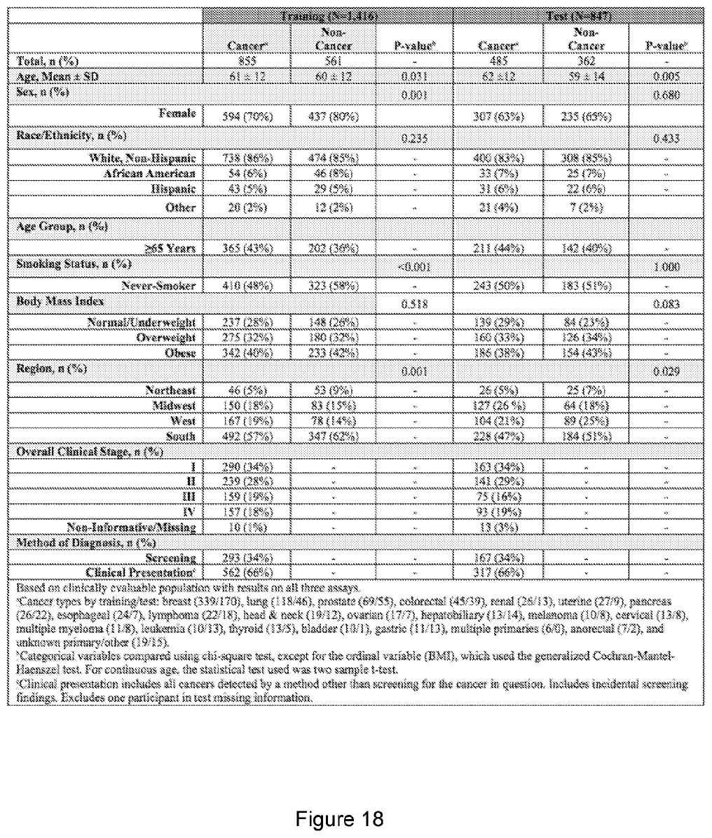

Subjects from the CCGA [NCT02889978] were used in the Examples of the present disclosure. CCGA is a prospective, multi-center, case-control, observational study with longitudinal follow-up. The study enrolled 9,977 of 15,000 demographically-balanced participants at 141 sites. Blood was collected from subjects with newly diagnosed therapy-naive cancer (C, case) and participants without a diagnosis of cancer (noncancer [NC], control) as defined at enrollment. This preplanned substudy included 1628 cases and 1172 controls, across twenty tumor types and all clinical stages. Samples were divided into training (1,785) and test (1,015) sets prior to analysis. Samples were selected to ensure a prespecified distribution of cancer types and non-cancers across sites in each cohort, and cancer and non-cancer samples were frequency age-matched by gender. FIG. 18 provides demographics of participants in the final analysis.

Cell-free DNA was isolated from plasma, while genomic DNA (gDNA) was isolated from white blood cells (WBCs) and tumor tissue using standard methodologies. Three distinct high-intensity sequencing approaches were employed in cfDNA analysis: (i) cfDNA whole-genome bisulfite sequencing (WGBS; 30× depth) in which normalized scores were generated using abnormally methylated fragments, (ii) paired cfDNA and WBC whole-genome sequencing (WGS; 30× depth) in which a novel machine learning algorithm generated cancer-related signal scores and joint analysis identified shared events, and (iii) paired cfDNA and WBC targeted sequencing (507-gene panel; 60,000× depth, referred to herein as the “ART” assay) in which a joint caller removed WBC-derived somatic variants and residual technical noise. WBC gDNA was subjected to targeted sequencing to identify clonal hematopoiesis (CH). Tumor tissue gDNA was subjected to WGS to identify somatic variants, which were used to calculate cfDNA tumor fraction.

In the targeted assay, non-tumor WBC-matched cfDNA somatic variants (SNVs/indels) accounted for 76% of all variants in NC and 65% in C. Consistent with somatic mosaicism (e.g., clonal hematopoiesis), WBC-matched variants increased with age; several were non-canonical loss-of-function mutations not previously reported. After WBC variant removal, canonical driver somatic variants were highly specific to C (e.g., in EGFR and PIK3CA, 0 NC had variants vs 11 and 30, respectively, of C). Similarly, of 8 NC with somatic copy number alterations (SCNAs) detected with WGS, four were derived from WBCs. WGBS data of the CCGA reveals informative hyper- and hypo-fragment level CpGs (1:2 ratio); a subset of which was used to calculate methylation scores. A consistent “cancer-like” signal was observed in <1% of NC participants across all assays (representing potential undiagnosed cancers). An increasing trend was observed in NC vs stages I-III vs stage IV (nonsyn. SNVs/indels per Mb [Mean±SD] NC: 1.01±0.86, stages I-III: 2.43±3.98; stage IV: 6.45±6.79; WGS score NC: 0.00±0.08, I-III: 0.27±0.98; IV: 1.95±2.33; methylation score NC: 0±0.50; I-III: 1.02±1.77; IV: 3.94±1.70). These data demonstrate the feasibility of achieving >99% specificity for invasive cancer, and support the promise of cfDNA assay for early cancer detection.

More information on the CCGA assay is disclosed in Klein et al., “Development of a comprehensive cell-free DNA (cfDNA) assay for early detection of multiple tumor types: The Circulating Cell-free Genome Atlas (CCGA) study,” 2018 ASCO Annual Meeting, Jun. 1-5, 2018, Abstract 12021 #134, Chicago, Ill., which is hereby incorporated by reference.

Determination of methylation alleles using WGBS. For each sample, the WGBS fragment set was reduced to a subset of unusual fragments of extreme methylation status (UFXM). Fragments occurring at high frequency in individuals without cancer, or that have unstable methylation, are unlikely to produce highly discriminatory features for classification of cancer status. A statistical model of typical fragments using an independent reference set of 108 non-smoking participants without cancer (age: 58±14 years, 79 [73%] women) from the CCGA study was used. These samples were used to train a Markov chain model (order 3) estimating the likelihood of a given sequence of CpG methylation statuses within a fragment. This model was demonstrated to be calibrated within the normal fragment range (p-value≥0.001) and was used to reject fragments with a p-value from the Markov model p≥0.001 as insufficiently unusual.

A further data reduction step selected only fragments with at least 5 CpGs covered, and average methylation per fragment either >0.9 (hyper methylated) or <0.1 (hypo-methylated). This procedure resulted in a median (range) of 2,800 (1,500-12,000) UFXM fragments for participants without cancer in training, and a median (range) of 3,000 (1,200-220,000) UFXM fragments for participants with cancer in training. As this data reduction procedure only used reference set data, this stage was only required to be applied to each sample once.

At selected loci within the genome, an approximate log-ratio score for informativeness for cancer status was constructed separately for both hyper- and hypo-methylated UFXM. First, for each sample at the locus a binary feature was generated: 0 if no UFXM fragment overlapped that locus within that sample, 1 if there existed a UFXM fragment overlapping the locus. The number of positive values (1s) in samples were then counted from participants with (C_{c}) and without cancer (C_{nc}). The log-ratio score was then constructed as: log(C_{c}+1)-log(C_{nc}+1), adding a regularization term to the counts, and discarding the normalization term relating to the total number of samples within each group (N_{c} and N_{nc}) as it is constant (log[N_{nc}+2]-log[N_{c}+2]). Scores were constructed at the locations of all CpG sites within the genome, resulting in approximately 25M loci with assigned scores: one score for UFXM hyper-methylated fragments and one score for UFXM hypo-methylated fragments.

Given a locus-specific log-ratio score, UFXM fragments in a sample were scored by taking the maximum of all log-ratio scores for loci within the fragment and matching the methylation category of either hyper- or hypo-methylated. This resulted in one score per UFXM fragment within a sample.

These fragment-level scores within a sample were reduced to a small set of features per sample by taking the scores of a subset of extreme-ranked fragments within each sample, separately for both hyper- and hypo-methylated fragments. In this way, information for the most informative fragments in each sample was captured using a small set of useful features. In a low cfDNA tumor fraction sample, only a minority of fragments were expected to be unusually informative.

In each category of fragments, the rank 1,2,4 . . . 64 (2ⁱ, i in 0:6) largest scores were selected for fragments within each category of hyper- and hypo-methylated UFXM, resulting in 14 features (7 and 7). To adjust for sample sequencing depth, the ranking procedure was treated as a function mapping ranks to scores, and interpolated between the observed scores was performed to obtain scores corresponding to adjusted ranks. The ranks were adjusted in linear proportion to relative sample depth: if the relative sample depth was x, interpolated scores were taken at x multiplied by the original ranks (e.g., for x=1.1, we take scores computed at ranks floor(1.1), floor(2.2), floor(x·2)). Every sample was then assigned a set of 14 adjusted extreme-rank scores to be used in further classification.

Given the feature vector, a kernel logistic regression classifier was used to capture potential non-linearities in predicting cancer/non-cancer status from the features. Specifically, a regularized kernel logistic regression classifier (KLR) was trained using the isotropic radial basis function (power exponential 2) as the kernel with scale parameter gamma, and L2 regularization parameter lambda (adjusted by dividing by m², where m is the number of samples so lambda scales naturally with the amount of training data). Gamma and lambda were optimized for holdout log-loss using internal cross-validation within specified training data, and were optimized using grid-search over the range 1-0.01 (gamma), 1000-10 (lambda) in 7 multiplicative steps, starting at the maximum value and halving the parameter each step. The median optimal parameters over internal cross-validation folds were 0.125 for gamma and 125 for lambda.

To evaluate performance of this extreme-rank-score classifier procedure on the CCGA substudy data set, cross-validation was applied to the training set, dividing the samples into 10 folds. Each fold was held out and the extreme rank score (ERS) classifier was trained on the remaining 9/10 of the data (using internal cross-validation within those folds to optimize gamma and lambda). The log-ratio scores used in featurization only accessed data from training folds. Output scores from each held-out fold were pooled and used to construct a ROC curve for performance.

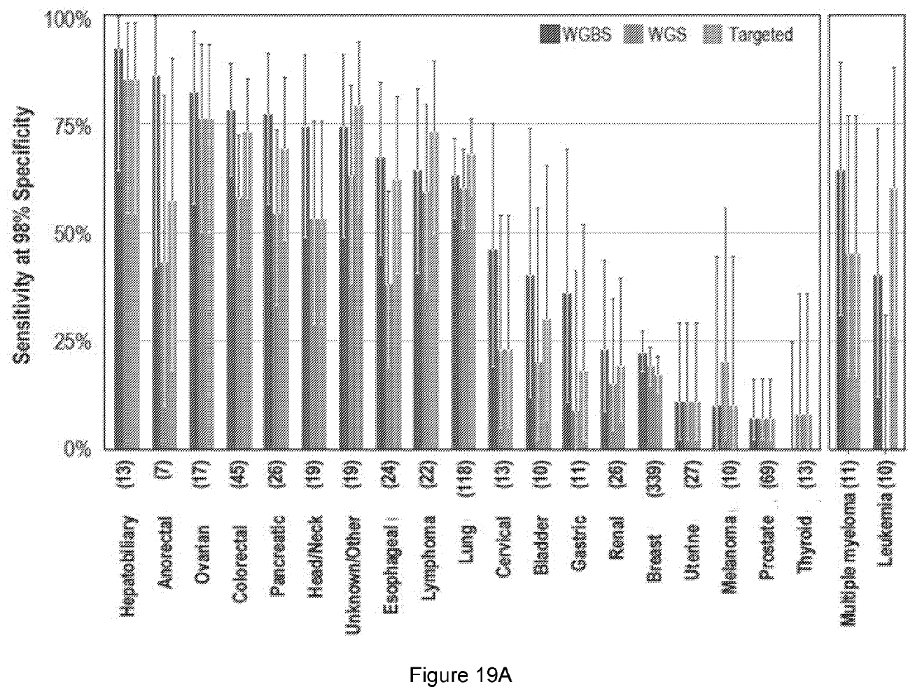

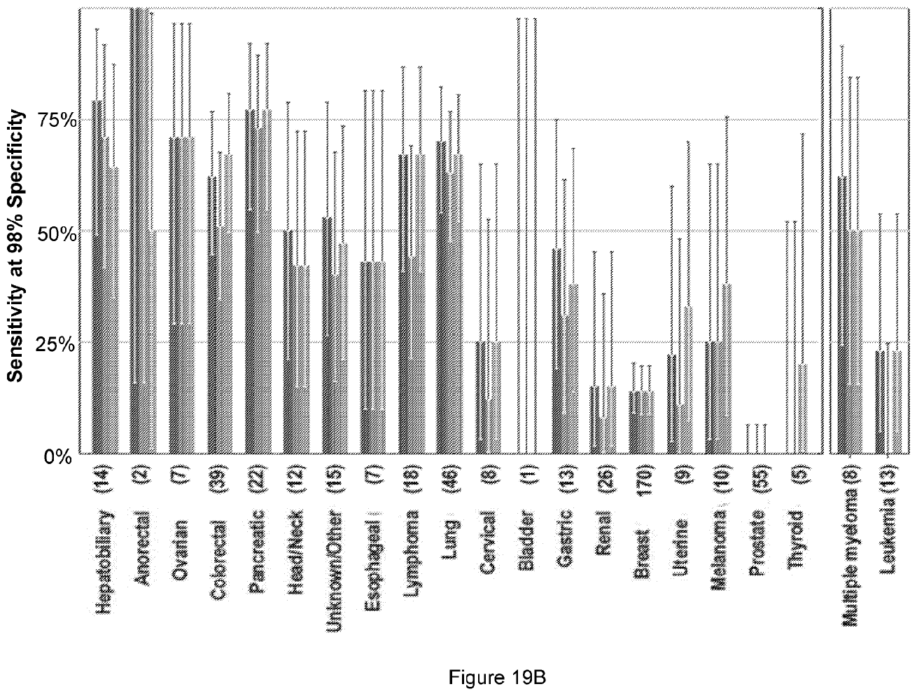

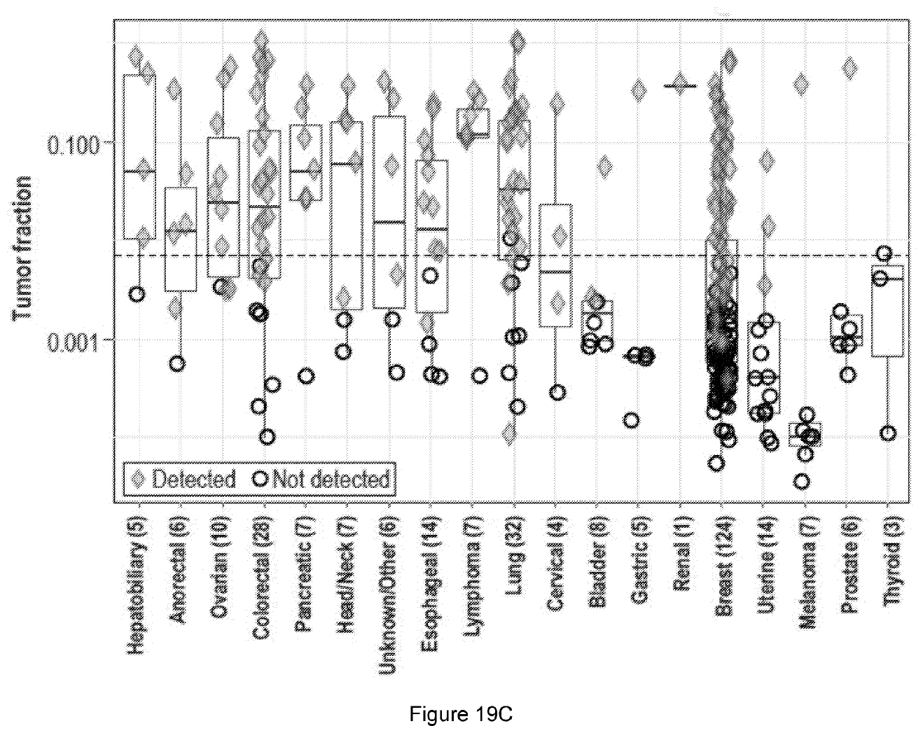

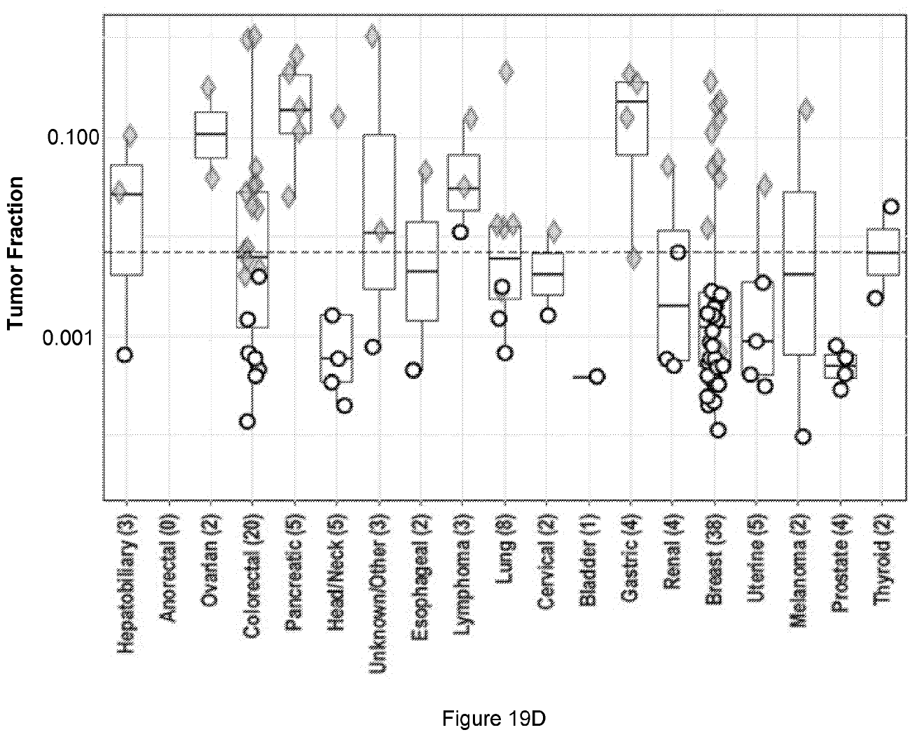

Sensitivity Estimates. FIGS. 19A and 19B provide information on sensitivity of models, trained using the training set summarized in FIG. 18, against both the training set (FIG. 19A, N=1,416) and the test set (FIG. 19B, N=847) summarized in FIG. 18, broken out by tumor of origin. FIGS. 19C and 19D provide information on tumor fraction in the training set (FIG. 19C) and the test set (FIG. 19D) broken out by tumor of origin. In more detail, for FIGS. 19A and 19B, sensitivity at 98% specificity (y-axis) for each tumor type (x-axis) in the training and test sets is given when analyzed by WGBS (left hand bars, blue), WGS accounting for CH (middle bars, orange), and the targeted assay accounting for CH (right hand bars, gray) in training (FIG. 19A) and test (FIG. 19B) sets. Error bars represent 95% confidence intervals. Number of samples per cancer type are indicated in parentheses. Multiple myeloma and leukemia from post hoc analyses are represented separately. FIGS. 19C and 19D provide box plots of cfDNA tumor fraction (y-axis) for a subset of participants with tumor-normal tissue sequencing available and at least one mutant cfDNA read (as indicated in parentheses), per tumor type (x-axis) in the training (FIG. 19C) and test (FIG. 19D) sets. Median as well as first and third quartiles are depicted. Individual participant cfDNA tumor fraction estimates are represented by diamonds (detected by WGBS at 98% specificity) and open circles (not detected by WGBS at 98% specificity). The horizontal dotted lines indicate the limit of detection (>50% probability of detection). FIG. 19D establishes that the disclosed methods can be used to detect a tumor fraction in cell free nucleic acid of a subject even when the tumor fraction f is 0.100 or less and, in many instance, when the tumor fraction f is 0.050 or less, 0.050 or less, 0.040 or less, 0.030 or less or even 0.020 or less in the subject.

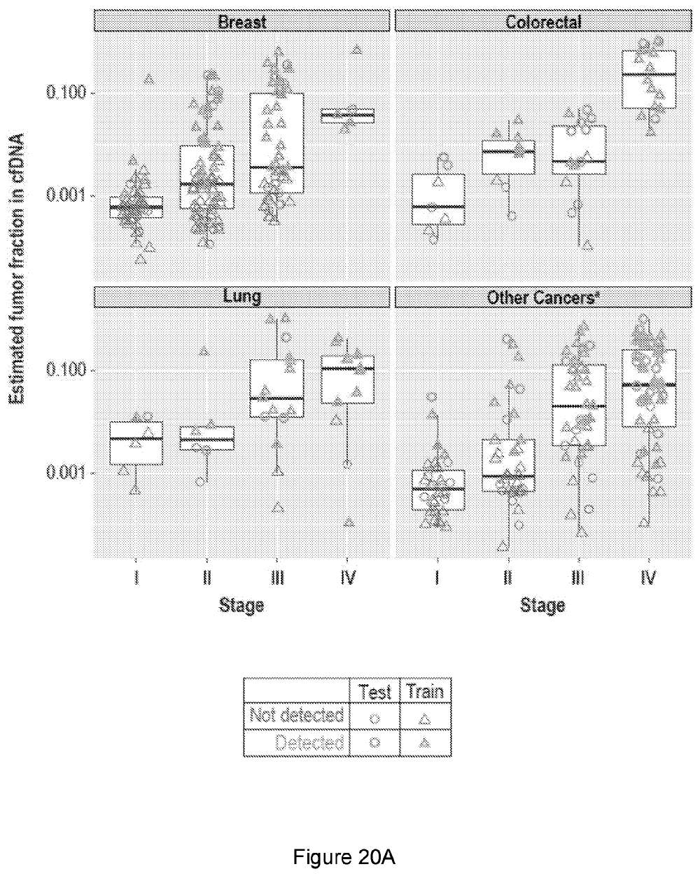

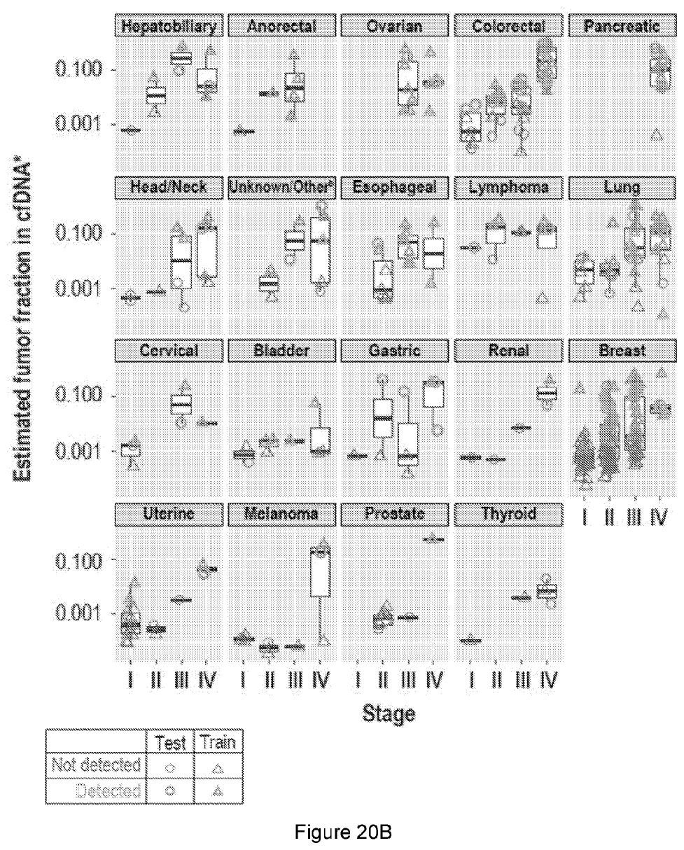

FIGS. 20A and 20B illustrate cfDNA tumor fraction as calculated by comparing cfDNA WGS with tumor WGS results by stage for breast cancer, colorectal cancer, lung cancer, and other cancers in aggregate (FIG. 20A), and by each cancer type (FIG. 20B). Samples with at least one mutant read in cfDNA are represented. Individual participant tumor fractions are indicated by triangles (training set) and circles (testing set), with symbol color indicating WGBS detection at 98% specificity (detected: blue; not detected: orange). FIG. 20A includes all non-breast, lung, and colorectal cancer samples. FIG. 20B includes two neuroendocrine, two mesothelioma, two gastrointestinal stromal tumor, one anal, and four adenocarcinomas (not otherwise specified) of unknown primary origin.

### EXAMPLE 13

Generation of Methylation State Vector

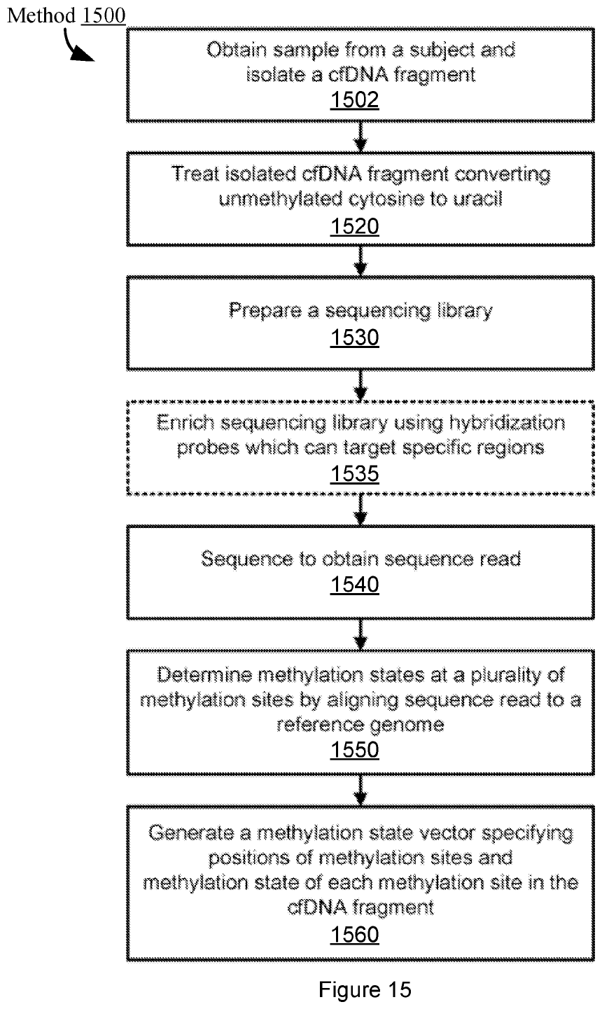

FIG. 15 is a flowchart describing a process 1500 of sequencing a fragment of cfDNA to obtain a methylation state vector, according to an embodiment in accordance with the present disclosure.

Referring to step 1502, the cfDNA fragments are obtained from the biological sample (e.g., as discussed above in conjunction with FIG. 2). Referring to step 1520, the cfDNA fragments are treated to convert unmethylated cytosines to uracils. In one embodiment, the DNA is subjected to a bisulfite treatment that converts the unmethylated cytosines of the fragment of cfDNA to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion in some embodiments. In other embodiments, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cfDNA fragments, a sequencing library is prepared (step 1530). Optionally, the sequencing library is enriched 1535 for cfDNA fragments, or genomic regions, that are informative for cancer status using a plurality of hybridization probes. The hybridization probes are short oligonucleotides capable of hybridizing to particularly specified cfDNA fragments, or targeted regions, and enriching for those fragments or regions for subsequent sequencing and analysis. Hybridization probes may be used to perform a targeted, high-depth analysis of a set of specified CpG sites of interest to the researcher. Once prepared, the sequencing library or a portion thereof can be sequenced to obtain a plurality of sequence reads (1540). The sequence reads may be in a computer-readable, digital format for processing and interpretation by computer software.

From the sequence reads, a location and methylation state for each of CpG site is determined based on alignment of the sequence reads to a reference genome (1550). A methylation state vector for each fragment specifying a location of the fragment in the reference genome (e.g., as specified by the position of the first CpG site in each fragment, or another similar metric), a number of CpG sites in the fragment, and the methylation state of each CpG site in the fragment (1560).

### Additional Embodiments

Using tumor fraction to evaluate performance of a classifier. Another aspect of the present disclosure provides for a method of evaluating a performance of a classifier. The method comprises obtaining in electronic form a respective dataset comprising a first plurality of sequence reads from a respective biological sample of a corresponding subject, for each subject in a plurality of subjects thereby obtaining a plurality of datasets. The respective biological sample of each corresponding subject comprises cell-free nucleic acid molecules from the corresponding subject. Each respective dataset in the plurality of datasets is applied to a classifier thereby obtaining a corresponding classifier result for the respective dataset. The classifier result indicates whether the corresponding subject in the plurality of subjects has a first cancer condition. Furthermore, prior to application of the above described datasets, the classifier is trained on data other than the plurality of datasets.

In some embodiments, an estimated tumor fraction in the cell free DNA of each subject in the plurality of subjects is estimated using the dataset corresponding to the subject. Then, a performance of the classifier is computed as a function of estimated tumor fraction across the plurality of subjects by comparing the classifier result for each respective subject to a clinical observation of the respective subject derived independent of the classifier versus the estimated tumor fraction of the respective subject.

Conditioning on tumor sequencing of an entire cohort, rather than just a single subject. Another aspect of the present disclosure provides a method of classifying a subject. The method comprises, at a computer system having one or more processors, and memory storing one or more programs for execution by the one or more processors, obtaining a first plurality of sequence reads in electronic form from a biological sample of the subject, where the biological sample comprises cell-free nucleic acid molecules. The first plurality of sequence reads of the cell-free nucleic acid molecules is used to identify support for each variant in a first variant set. A respective sequence read in the first plurality of sequence reads is deemed to support a variant in the first variant set when the respective sequence read contains all or a portion of the variant. A respective sequence read in the first plurality of sequence reads is deemed to not support a variant in the first variant set when the respective sequence read does not contain all or a portion of the variant. In this way, an observed frequency of each variant in the first variant set is determined from among the sequence reads in the first plurality of sequence reads that do support and do not support each variant in the first variant set. The observed frequency of each variant in the first variant set is compared to a corresponding reference frequency in a first reference set. Each corresponding reference frequency in the first reference set is a frequency of the corresponding variant across a first plurality of aberrant tissue samples of a common (same) first class. The subject is then classified. This classifying comprises deeming the subject to have a first condition associated with the first plurality of aberrant tissue samples when the observed frequency of each variant in the first variant set satisfies a first threshold. The first threshold is determined by each reference frequency in the first reference set.

In some embodiments, the first condition is a cancer from a common primary site of origin. In some embodiments, the first condition is a cancer from two or more common primary sites of origin. In some embodiments, the first condition is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, the first condition is a predetermined stage of a breast cancer, lung cancer, prostate cancer, colorectal cancer, renal cancer, uterine cancer, pancreatic cancer, cancer of the esophagus, lymphoma, head/neck cancer, ovarian cancer, hepatobiliary cancer, melanoma, cervical cancer, multiple myeloma, leukemia, thyroid cancer, bladder cancer, or gastric cancer.

In some embodiments, the first condition is a predetermined subtype of a cancer (e.g., breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer).

In some embodiments, a variant in the first variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

In some embodiments, the first plurality of aberrant tissue samples are tumor samples. In some embodiments the first variant set consists of a single variant that is a single genetic variation at a single locus in the genome of the subject. In some embodiments the first variant set consists of a first variant that is a first genetic variation at a first locus in the genome of the subject and a second variant that is a second genetic variation at a second locus in the genome of the subject.

In some embodiments the first variant set consists of: a first variant that is a first genetic variation at a first locus in the genome of the subject, a second variant that is a second genetic variation at a second locus in the genome of the subject, and a third variant that is a third genetic variation at a third locus in the genome of the subject.

In some embodiments the first variant set consists of between two and twenty variants, where each variant in the first variant set is a different genetic variation (and optionally at a different locus) in the genome of the subject. In some embodiments the first variant set consists of between 2 and 200 variants, where each variant in the first variant set is a different genetic variation (and optionally at a different locus) in the genome of the subject.

In some embodiments the first variant set comprises 200 variants, comprises 300 variants, comprises 400 variants, comprises 500 variants, comprises 750 variants, comprises 1000 variants, comprises 2000 variants, or comprises 5000 variants where each variant in the first variant set is a different genetic variation (and optionally at a different locus) in the genome of the subject.

In some embodiments the comparing comprises computing a single estimated ctDNA fraction in the cfDNA of the human subject from the observed frequency of each variant in the first variant set. In such embodiments, the first threshold is a single expected ctDNA fraction in the cfDNA of the human subject that is determined from the value of each reference frequency in the first reference set. In some such embodiments, the single expected ctDNA fraction in the cfDNA is between 0.5×10⁻⁴ and 1.5×10⁻⁴, and the first condition is a melanoma. In some such embodiments, the single expected ctDNA fraction in the cfDNA is between 0.5×10⁻³ and 1×10⁻², and the first condition is a renal cancer, uterine cancer, thyroid cancer, prostate cancer, breast cancer, bladder cancer, gastric cancer, cervical cancer, or a combination thereof. In some such embodiments, the single expected ctDNA fraction in the cfDNA is between 1×10⁻² and 0.8, and the first condition is lung cancer, esophageal cancer, a head/neck cancer, colorectal cancer, anorectal cancer, ovarian cancer, a hepatobiliary cancer, a pancreatic cancer, a lymphoma, or a combination thereof.

In some embodiments the using comprises aligning a respective sequence read in the first plurality of sequence reads to a region in a reference genome in order to determine whether the respective sequence read contains all or a portion of a variant. In some embodiments the using comprises aligning a respective sequence read in the first plurality of sequence reads to a lookup table of variants in order to determine whether the sequence read contains all or a portion of a variant. In some embodiments the using comprises aligning a sequence read in the first plurality of sequence reads to each entry in a lookup table, where each entry in the lookup table represents a different portion of a genome.

In some embodiments, the comparing comprises computing a single estimated circulating tumor DNA (ctDNA) fraction in the cell free DNA (cfDNA) of the human subject from the observed frequency of each variant in the first variant set. In such embodiments, the observed frequency of each variant in the first variant set satisfies the first threshold when the single estimated circulating tumor DNA (ctDNA) fraction exceeds 1×10′, and the first condition is stage II, stage III, or stage IV breast cancer.

In some embodiments, the method further comprises using the first plurality of sequence reads to identify support for each variant in a second variant set, where a respective sequence read in the first plurality of sequence reads is deemed to support a variant in the second variant set when the respective sequence read contains all or a portion of the second variant, and a respective sequence read in the first plurality of sequence reads is deemed to not support a variant in the second variant set when the respective sequence read does not contain the respective second variant. In this way, an observed frequency of each variant in the second variant set is determined from among the sequence reads in the first plurality of sequence reads that do support and do not support a variant in the second variant set. This observed frequency of each variant in the second variant set is compared to a corresponding second reference frequency in a second reference set. In such embodiments, each corresponding second reference frequency in the second reference set is a frequency of the corresponding variant across a second plurality of aberrant tissue samples of a common (same) second class. Further, in such embodiments, the classifying the human subject further comprises deeming the human subject to have a second condition associated with the second plurality of aberrant tissue samples when the observed frequency of each variant in the second variant set satisfies a second threshold, where the second threshold is determined by each reference frequency in the second reference set.

In some embodiments the subject is a human subject. In some embodiments the biological sample comprises blood, whole blood, plasma, serum, urine, cerebrospinal fluid, fecal, saliva, sweat, tears, pleural fluid, pericardial fluid, or peritoneal fluid of the subject.

Another aspect of the present disclosure provides a computing system, comprising one or more processors, and memory storing one or more programs to be executed by the one or more processors. The one or more programs comprise instructions for classifying a subject by a method. The method comprises (A) obtaining a first plurality of sequence reads in electronic form from a biological sample of the subject, where the biological sample comprises cell-free nucleic acid molecules. The method further comprises (B) using the first plurality of sequence reads of the cell-free nucleic acid molecules to identify support for each variant in a first variant set. Here, a respective sequence read in the first plurality of sequence reads is deemed to support a variant in the first variant set when the respective sequence read contains all or a portion of the variant, and a respective sequence read in the first plurality of sequence reads is deemed to not support a variant in the first variant set when the respective sequence read does not contain the variant. In this way, an observed frequency of each variant in the first variant set is determined from among the sequence reads in the first plurality of sequence reads that do support and do not support each variant in the first variant set. The observed frequency of each variant in the first variant set is compared to a corresponding reference frequency in a first reference set. In such embodiments, each corresponding reference frequency in the first reference set is a frequency of the corresponding variant across a first plurality of aberrant tissue samples of a common (same) first class. The subject is then classified. This classifying comprises deeming the subject to have a first condition associated with the first plurality of aberrant tissue samples when the observed frequency of each variant in the first variant set satisfies a first threshold, where the first threshold is determined by each reference frequency in the first reference set.

Another aspect of the present disclosure provides a non-transitory computer readable storage medium storing one or more programs for classifying a subject. The one or more programs are configured for execution by a computer. The one or more programs comprise instructions for obtaining a first plurality of sequence reads in electronic form from a biological sample of the subject, where the biological sample comprises cell-free nucleic acid molecules. The one or more programs further comprise instructions for using the first plurality of sequence reads of the cell-free nucleic acid molecules to identify support for each variant in a first variant set. In such embodiments, a respective sequence read in the first plurality of sequence reads is deemed to support a variant in the first variant set when the respective sequence read contains all or a portion of the variant, and a respective sequence read in the first plurality of sequence reads is deemed to not support a variant in the first variant set when the respective sequence read does not contain the variant. In this way, an observed frequency of each variant in the first variant set is determined from among the sequence reads in the first plurality of sequence reads that do support and do not support each variant in the first variant set. The observed frequency of each variant in the first variant set is compared to a corresponding reference frequency in a first reference set. In such embodiments, each corresponding reference frequency in the first reference set is a frequency of the corresponding variant across a first plurality of aberrant tissue samples of a common (same) first class. The one or more programs further comprise instructions for classifying the subject. The classifying comprises deeming the subject to have a first condition associated with the first plurality of aberrant tissue samples when the observed frequency of each variant in the first variant set satisfies a first threshold. Here, the first threshold is determined by each reference frequency in the first reference set.

Estimation of cfDNA tumor fraction from read information from an individual without analyzing the aberrant tissue directly.

In alternative embodiments, cfDNA tumor fraction is estimated without using sequence reads 126 from an aberrant tissue. In some such approaches, tumor derived features (e.g. small variants) are identified using sequence reads 140 from the biological sample containing the cell free nucleic acid. Then conditional upon the observed frequency of one of these variants, the underlying tumor fraction is estimated.

In some such embodiments, in order to ensure that a given mutation is a suitable surrogate for single estimated ctDNA fraction in the cfDNA of the subject, the selected variant is a variant that has other than the highest frequency on the presumed basis that this variant has a high probability of not originating from the aberrant tissue. To illustrate, consider the case where the cell free nucleic acid of a biological sample is sequenced and a first variant 130-1 with a first frequency 132-1 and a second variant 130-2 with a second reference frequency 132-2 are found, where the first reference frequency 132-1 is greater than the second reference frequency 132-2. In this instance, only the second variant 132-2 is presumed to be a suitable surrogate of the condition associated with the unmeasured aberrant tissue of the given subject.

In some such embodiments, in order to ensure that a given mutation is a suitable surrogate for single estimated ctDNA fraction in the cfDNA of the subject, variants that are known to not be associated with the condition under study (e.g., variants that are often associated with white blood cells) are excluded from consideration.

In some embodiments a respective variant 144 is used for estimating tumor fraction on the basis that the respective variant 144 has the second highest frequency of all the variants observed in the biological sample containing the cell free nucleic acid (e.g., blood sample). For instance, if the frequency of this variant (number of observed sequence reads covering the position of the variant in the genome that support the variant divided the total number of observed sequence reads covering the position of the variant in the genome) is ten percent, then the single estimated ctDNA fraction in the cfDNA of the subject is ten percent.

In some embodiments a respective variant 144 in the first variant set 142 is used for estimating the tumor fraction on the basis that it has the third highest frequency of all the variants observed in the biological sample containing the cell free nucleic acid (e.g., blood sample). For instance, if the frequency of this variant (number of observed sequence reads covering the position of the variant in the genome that support the variant divided the total number of observed sequence reads covering the position of the variant in the genome) is ten percent, then the single estimated ctDNA fraction in the cfDNA of the subject is ten percent.

In some such embodiments, the embodiment that does not make use of the aberrant tissue sample and rather just uses the biological sample containing the cell free nucleic acid is useful for computing single estimated tumor fractions down to about one percent.

In some embodiments the second highest ranking variant by frequency is used as a proxy of true tumor fraction (single estimated ctDNA fraction in the cfDNA of the subject). For instance, if the frequency of this variant (number of observed sequence reads covering the position of the variant in the genome that support the variant divided the total number of observed sequence reads covering the position of the variant in the genome) is ten percent, then the single estimated ctDNA fraction in the cfDNA of the subject is ten percent.

In some embodiments the third highest ranking variant by frequency is used as a proxy of true tumor fraction (single estimated ctDNA fraction in the cfDNA of the subject). For instance, if the frequency of this variant (number of observed sequence reads covering the position of the variant in the genome that support the variant divided the total number of observed sequence reads covering the position of the variant in the genome) is ten percent, then the single estimated ctDNA fraction in the cfDNA of the subject is ten percent.

In some embodiments, the single estimated ctDNA fraction in the cfDNA of the subject from the biological sample containing cell free nucleic acid serves as a reference basis for biological samples taken from the same subject at later time points in order to determine a change in the tumor fraction in the subject over time.

### CONCLUSION

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed:

1. A method of determining tumor fraction in cell-free nucleic acid of a liquid biological sample of a subject, the method comprising:
at a computer system having one or more processors, and memory storing one or more programs for execution by the one or more processors:
(A) obtaining a first plurality of sequence reads in electronic form from the liquid biological sample of the subject, wherein the liquid biological sample comprises cell-free nucleic acid molecules;
(B) using the first plurality of sequence reads to identify support for each variant in a first variant set thereby determining an observed frequency of each variant in the first variant set;
(C) for each respective variant in the first variant set, obtaining a corresponding reference frequency for the respective variant in a first reference set, wherein each corresponding reference frequency in the first reference set is for a respective variant in a first aberrant solid tissue sample obtained from the subject; and
(D) evaluating the observed frequency of each respective variant in the first variant set against the observed frequency of the respective variant in the first reference set in the first aberrant solid tissue thereby determining a first tumor fraction in cell-free nucleic acid of the liquid biological sample of the subject.

2. The method of claim 1, wherein a variant in the first variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

3. The method of claim 1, wherein
a respective sequence read in the first plurality of sequence reads is deemed to support a first variant in the first variant set when the respective sequence read contains all or a portion of the first variant, and
a respective sequence read in the first plurality of sequence reads is deemed to not support the first variant in the first variant set when the respective sequence read does not contain the first variant, and
a number of sequence reads in the first plurality of sequence reads that support the first variant versus a number of sequence reads in the first plurality of sequence reads that do not support the first variant determine the observed frequency of the first variant, which estimates the variant frequency of the first variant within the liquid biological sample.

4. The method of claim 1, wherein the subject is human.

5. The method of claim 1, wherein the subject has a cancer from a single primary site of origin.

6. The method of claim 1, wherein the subject has a cancer originating from two or more different organs.

7. The method of claim 1, wherein the subject has breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

8. The method of claim 1, wherein the subject has a predetermined stage of breast cancer, lung cancer, prostate cancer, colorectal cancer, renal cancer, uterine cancer, pancreatic cancer, cancer of the esophagus, head/neck cancer, ovarian cancer, hepatobiliary cancer, cervical cancer, thyroid cancer, bladder cancer, or gastric cancer.

9. The method of claim 1, wherein the first aberrant solid tissue sample is a tumor sample.

10. The method of claim 1, wherein the first variant set consists of a single variant for a single genetic variation at a single locus in the genome of the subject.

11. The method of claim 1, wherein the first variant set consists of a first variant for a first genetic variation at a first locus in the genome of the subject and a second variant for a second genetic variation at a second locus in the genome of the subject.

12. The method of claim 1, wherein the first variant set consists of:
a first variant for a first genetic variation at a first locus in the genome of the subject,
a second variant for a second genetic variation at a second locus in the genome of the subject, and
a third variant for a third genetic variation at a third locus in the genome of the subject.

13. The method of claim 1, wherein the first variant set consists of between two and twenty variants, and wherein each variant in the first variant set is for a different genetic variation in the genome of the subject.

14. The method of claim 1, wherein the first variant set consists of between 2 and 200 variants, and wherein each variant in the first variant set is for a different genetic variation in the genome of the subject.

15. The method of claim 1, wherein the first variant set comprises 1000 variants, and wherein each variant in the first variant set is for a different genetic variation in the genome of the subject.

16. The method of claim 1, wherein the first variant set comprises 5000 variants, and wherein each variant in the first variant set is for a different genetic variation in the genome of the subject.

17. The method of claim 1, wherein the using (B) comprises aligning a sequence read in the first plurality of sequence reads to a region in a reference genome in order to determine whether the sequence read contains all or a portion of a first variant.

18. The method of claim 1 wherein the using (B) comprises aligning a sequence read in the first plurality of sequence reads to a lookup table of variants in order to determine whether the sequence read contains all or a portion of a first variant.

19. The method of claim 1, wherein the using (B) comprises aligning a sequence read in the first plurality of sequence reads to each entry in a lookup table, wherein each entry in the lookup table represents a different portion of a genome.

20. The method of claim 1, wherein the subject has stage II, stage III, or stage IV breast cancer and the evaluating (D) determines that the first tumor fraction of the cell-free nucleic acid is less than 1×10⁻³.

21. The method of claim 1, the method further comprising:
using the first plurality of sequence reads to identify support for each variant in a second variant set thereby determining an observed frequency of each variant in the second variant set;
for each respective variant in the second variant set, obtaining a corresponding reference frequency for the respective variant in a second reference set, wherein each corresponding reference frequency in the second reference set is for a respective variant in a second aberrant solid tissue sample obtained from the subject; and
evaluating the observed frequency of each respective variant in the second variant set against the observed frequency of the respective variant in the second reference set, thereby determining a second tumor fraction in cell-free nucleic acid of the liquid biological sample of the subject.

22. The method of claim 21, wherein
a respective sequence read in the first plurality of sequence reads is deemed to support a variant in the second variant set when the respective sequence read contains all or a portion of the variant, and
a respective sequence read in the first plurality of sequence reads is deemed to not support a variant in the second variant set when the respective sequence read does not contain the variant.

23. The method of claim 21, wherein the first aberrant tissue sample consists of a first tumor fraction and the second aberrant tissue sample consists of a second tumor fraction of the same tumor from the subject.

24. The method of claim 21, wherein the first aberrant tissue sample is of a first cancer type and the second aberrant tissue sample is of a second cancer type.

25. The method of claim 24, wherein the first cancer type is the same as the second cancer type.

26. The method of claim 24, wherein the first cancer type is other than the second cancer type.

27. The method of claim 26, wherein the first cancer type and the second cancer type are each selected from the group consisting of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, and gastric cancer.

28. The method of claim 1, wherein the frequency of each variant in the first reference set is obtained from a second plurality of sequence reads collectively taken from the first aberrant solid tissue sample.

29. The method of claim 28, wherein more than 1000 sequence reads are collectively taken from the first aberrant solid tissue sample.

30. The method of claim 28, wherein more than 3000 sequence reads are collectively taken from the first aberrant solid tissue sample.

31. The method of claim 28, wherein more than 5000 sequence reads are collectively taken from the first aberrant solid tissue sample.

32. The method of claim 28, wherein the method further comprises analyzing the second plurality of sequence reads taken from the first aberrant solid tissue sample against a panel of variant candidates.

33. The method of claim 32, wherein the panel of variant candidates comprises between one hundred variants and one thousand variants.

34. The method of claim 28, wherein the second plurality of sequence reads taken from the first aberrant solid tissue sample represents whole genome data for the respective cell.

35. The method of claim 34, wherein an average coverage rate of the second plurality of sequence reads taken from the first aberrant solid tissue sample is at least 10×.

36. The method of claim 34, wherein an average coverage rate of the second plurality of sequence reads taken from the first aberrant solid tissue sample is at least 100×.

37. The method of claim 34, wherein an average coverage rate of the second plurality of sequence reads taken from the first aberrant solid tissue sample is at least 2000×.

38. The method of claim 1, wherein the liquid biological sample comprises blood, whole blood, plasma, serum, urine, cerebrospinal fluid, fecal, saliva, sweat, tears, pleural fluid, pericardial fluid, or peritoneal fluid of the subject.

39. The method of claim 1, wherein the liquid biological sample consists of blood, whole blood, plasma, serum, urine, cerebrospinal fluid, fecal, saliva, sweat, tears, pleural fluid, pericardial fluid, or peritoneal fluid of the subject.

40. The method of claim 1, wherein the evaluating the observed frequency of each respective variant in the first variant set to a corresponding reference frequency for the respective variant in the first reference set (C) comprises evaluating a cumulative density function or a cumulative distribution function for the respective variant using the observed frequency and the reference frequency for the respective variant across a range of possible tumor fractions.

41. The method of claim 40, wherein a cumulative density function is used.

42. The method of claim 41, wherein the range is zero percent to 110 percent.

43. The method of claim 41 or 42, wherein the first tumor fraction is deemed to be a median value of the cumulative density function.

44. The method of claim 40, wherein a cumulative distribution function is used.

45. The method of claim 44, wherein the cumulative distribution function has the form:
$$P ( x ; p , n ) = {\sum }_{i = 0}^{x} \frac{n !}{i ! ( n - i ) !} {( p )}^{i} {( i - p )}^{( n - i )}$$
wherein,
x=a₂ᵢ, the observed number of sequence reads that support the respective variant in the liquid biological sample,
p=t*f₁ᵢ, wherein t is the estimated first tumor fraction, and f₁ᵢ is the observed frequency of the respective variant in the first variant set, and
n=d₂ᵢ, the total number of sequence reads from the biological sample mapping to the genomic location corresponding to the respective variant

46. The method of claim 44, wherein the cumulative distribution function has the form:
$$log P ( {x}_{k} ; {p}_{k} , {n}_{k} ) = {\sum }_{k} log ( {\sum }_{i = 0}^{x} \frac{{n}_{k} !}{i ! ( {n}_{k} - i ) !} {( {p}_{k} )}^{i} {( i - {p}_{k} )}^{( {n}_{k} - i )} )$$
wherein,
xₖ=a₂ᵢ, the observed number of sequence reads that support the respective variant k in the liquid biological sample,
pₖ=t*f₁ᵢ, wherein t is the estimated first tumor fraction, and f₁ᵢ is the observed frequency of the respective variant kin the first variant set, and
nₖ=d₂ᵢ, the total number of sequence reads from the biological sample mapping to the genomic location corresponding to the respective variant k.

47. The method of claim 40, wherein the cumulative density function or the cumulative distribution function is drawn under a negative binomial distribution assumption.

48. The method of claim 1, the method further comprising:
(E) repeating the obtaining (A) at each respective time point in a plurality of time points across an epoch, from a respective biological sample of the subject taken at each respective time point, wherein the respective biological sample comprises cell-free nucleic acid molecules, thereby obtaining a corresponding first plurality of sequence reads for the subject at each respective time point;
(F) determining, for each respective time point in the plurality of time points, support for each variant in the first variant set in the corresponding first plurality of sequence reads for the subject at the respective time point, thereby determining an observed frequency of each respective variant in the first variant set from among the sequence reads in the corresponding first plurality of sequence reads that do support and do not support the respective variant at each time point in the plurality of time points; and
(G) evaluating the observed frequency of each respective variant in the first variant set at each time point in the plurality of time points against the observed frequency of the respective variant in the first reference set in the first aberrant solid tissue thereby determining the state or progression of a disease condition in the subject during the epoch in the form of an increase or decrease of the first tumor fraction over the epoch.

49. The method of claim 48, wherein the epoch is a period of months and each time point in the plurality of time points is a different time point in the period of months.

50. The method of claim 49, wherein the period of months is less than four months.

51. The method of claim 48, wherein the epoch is a period of years and each time point in the plurality of time points is a different time point in the period of years.

52. The method of claim 51, wherein the period of years is between two and ten years.

53. The method of claim 48, wherein the epoch is a period of hours and each time point in the plurality of time points is a different time point in the period of hours.

54. The method of claim 53, wherein the period of hours is between one hour and six hours.

55. The method of claim 48, the method further comprising changing a diagnosis of the subject when the first tumor fraction of the subject is observed to change by a threshold amount across the epoch.

56. The method of claim 48, further comprising changing a prognosis of the subject when the first tumor fraction of the subject is observed to change by a threshold amount across the epoch.

57. The method of claim 48, further comprising changing a treatment of the subject when the first tumor fraction of the subject is observed to change by a threshold amount across the epoch.

58. The method of claim 48, wherein the disease condition is a cancer.

59. The method of claim 58, wherein the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof.

60. The method of claim 48, wherein the disease condition is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

61. The method of claim 48, wherein the disease condition is a predetermined subtype of a cancer.

62. The method of claim 1, the method further comprising:
(E) applying the first plurality of sequence reads to a trained classifier thereby obtaining a classifier result, wherein the trained classifier result indicates whether the subject has a first cancer condition; and
(G) using the trained classifier result as a basis for diagnosis or prognosis of the subject for the first cancer condition when the first tumor fraction is between 0.003 and 1.0 and the trained classifier result indicates that the subject has the first cancer condition.

63. The method of claim 62, wherein the first cancer condition is a cancer.

64. The method of claim 63, wherein the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof.

65. The method of claim 62, wherein the first cancer condition is a subtype of a cancer.

66. The method of claim 65, wherein the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

67. The method of claim 62, wherein the first tumor fraction is between 0.003 and 1.0 and the first cancer condition is a tissue of origin of a cancer.

68. The method of claim 62, wherein the trained classifier is a neural network, a support vector machine, a decision tree, an unsupervised clustering model, a supervised clustering model, or a regression model.

69. The method of claim 1 wherein the subject has a tumor fractionf of 0.100 or less.

70. The method of claim 1 wherein the subject has a tumor fractionf of 0.050 or less.

71. A computing system, comprising:
one or more processors;
memory storing one or more programs to be executed by the one or more processors;
the one or more programs comprising instructions for determining tumor fraction in cell-free nucleic acid of a liquid biological sample of a subject by a method comprising:
(A) obtaining a first plurality of sequence reads in electronic form from the liquid biological sample of the subject, wherein the liquid biological sample comprises cell-free nucleic acid molecules;
(B) using the first plurality of sequence reads to identify support for each variant in a first variant set thereby determining an observed frequency of each variant in the first variant set;
(C) for each respective variant in the first variant set, obtaining a corresponding reference frequency for the respective variant in a first reference set, wherein each corresponding reference frequency in the first reference set is for a respective variant in a first aberrant solid tissue sample obtained from the subject; and
(D) evaluating the observed frequency of each respective variant in the first variant set against the observed frequency of the respective variant in the first reference set in the first aberrant solid tissue thereby determining a first tumor fraction in cell-free nucleic acid of the liquid biological sample of the subject.

72. A non-transitory computer readable storage medium storing one or more programs determining tumor fraction in cell-free nucleic acid of a liquid biological sample of a subject, the one or more programs configured for execution by a computer, the one or more programs comprising instructions for:
(A) obtaining a first plurality of sequence reads in electronic form from the liquid biological sample of the subject, wherein the liquid biological sample comprises cell-free nucleic acid molecules;
(B) using the first plurality of sequence reads to identify support for each variant in a first variant set thereby determining an observed frequency of each variant in the first variant set;
(C) for each respective variant in the first variant set, obtaining a corresponding reference frequency for the respective variant in a first reference set, wherein each corresponding reference frequency in the first reference set is for a respective variant in a first aberrant solid tissue sample obtained from the subject; and
(D) evaluating the observed frequency of each respective variant in the first variant set against the observed frequency of the respective variant in the first reference set in the first aberrant solid tissue thereby determining a first tumor fraction in cell-free nucleic acid of the liquid biological sample of the subject.

73. A method of determining tumor fraction in cell-free nucleic acid of a liquid biological sample of a subject, the method comprising:
at a computer system having one or more processors, and memory storing one or more programs for execution by the one or more processors:
(A) obtaining a plurality of sequence reads in electronic form from the liquid biological sample of the subject, wherein the liquid biological sample comprises cell-free nucleic acid molecules;
(B) using the plurality of sequence reads to identify support for each variant in a variant set thereby determining an observed frequency of each variant in the first variant set; and
(C) deeming the observed frequency of the variant having the N^{th} highest allele frequency in the variant set to be the tumor fraction in cell-free nucleic acid of the liquid biological sample of the subject, wherein N is a positive integer other than one.

74. The method of claim 73, wherein N is 2.

75. The method of claim 73, wherein N is 3.

76. The method of claim 73, wherein a variant in the variant set is a single nucleotide variant associated with a predetermined genomic location, an insertion mutation associated with a predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location.

77. The method of claim 73, wherein
a respective sequence read in the plurality of sequence reads is deemed to support a first variant in the variant set when the respective sequence read contains all or a portion of the first variant, and
a respective sequence read in the plurality of sequence reads is deemed to not support the first variant in the variant set when the respective sequence read does not contain the first variant, and
a number of sequence reads in the plurality of sequence reads that support the first variant versus a number of sequence reads in the plurality of sequence reads that do not support the first variant determine the observed frequency of the first variant, which estimates the variant frequency of the first variant within the liquid biological sample.

78. The method of claim 73, wherein the subject has a cancer from a single primary site of origin.

79. The method of claim 73, wherein the subject has a cancer originating from two or more different organs.

80. The method of claim 73, wherein the subject has breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

81. The method of claim 73, wherein the variant set comprises five or more variants, and wherein each respective variant in the variant set is at a different locus in the genome of the subject.

82. The method of claim 73, wherein the variant set consists of between three and twenty variants, and wherein each variant in the variant set is for a different genetic variation in the genome of the subject.

83. The method of claim 73, wherein the variant set consists of between 2 and 200 variants, and wherein each variant in the variant set is for a different genetic variation in the genome of the subject.

84. The method of claim 73, wherein the variant set comprises 1000 variants, and wherein each variant in the variant set is for a different genetic variation in the genome of the subject.

85. The method of claim 73, wherein the using (B) comprises aligning a sequence read in the plurality of sequence reads to a region in a reference genome in order to determine whether the sequence read contains all or a portion of a first variant.

86. The method of claim 73, wherein the using (B) comprises aligning a sequence read in the plurality of sequence reads to a lookup table of variants in order to determine whether the sequence read contains all or a portion of a first variant.

87. The method of claim 73, wherein the using (B) comprises aligning a sequence read in the plurality of sequence reads to each entry in a lookup table, wherein each entry in the lookup table represents a different portion of a genome.

88. The method of claim 73, wherein the liquid biological sample comprises blood, whole blood, plasma, serum, urine, cerebrospinal fluid, fecal, saliva, sweat, tears, pleural fluid, pericardial fluid, or peritoneal fluid of the subject.

89. The method of claim 73, wherein the biological sample consists of blood, whole blood, plasma, serum, urine, cerebrospinal fluid, fecal, saliva, sweat, tears, pleural fluid, pericardial fluid, or peritoneal fluid of the subject.

90. The method of claim 73, the method further comprising:
(D) repeating the obtaining (A) at each respective time point in a plurality of time points across an epoch, from a respective biological sample of the subject taken at each respective time point, wherein the respective biological sample comprises cell-free nucleic acid molecules, thereby obtaining a corresponding plurality of sequence reads for the subject at each respective time point; and
(E) determining, for each respective time point in the plurality of time points, support for the variant in the variant set that had the N^{th} highest allele frequency in the deeming (C), thereby determining the state or progression of a disease condition in the subject during the epoch in the form of an increase or decrease of the allele frequency of the variant over the epoch.

91. The method of claim 90, wherein the epoch is a period of months and each time point in the plurality of time points is a different time point in the period of months.

92. The method of claim 91, wherein the period of months is less than four months.

93. The method of claim 90, wherein the epoch is a period of years and each time point in the plurality of time points is a different time point in the period of years.

94. The method of claim 93, wherein the period of years is between two and ten years.

95. The method of claim 90, wherein the epoch is a period of hours and each time point in the plurality of time points is a different time point in the period of hours.

96. The method of claim 95, wherein the period of hours is between one hour and six hours.

97. The method of claim 90, the method further comprising changing a diagnosis of the subject when the allele frequency of the variant is observed to change by a threshold amount across the epoch.

98. The method of claim 90, further comprising changing a prognosis of the subject when the allele frequency of the variant is observed to change by a threshold amount across the epoch.

99. The method of claim 90, further comprising changing a treatment of the subject when the allele frequency of the variant is observed to change by a threshold amount across the epoch.

100. The method of claim 90, wherein the disease condition is a cancer.

101. The method of claim 100, wherein the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof.

102. The method of claim 90, wherein the disease condition is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

103. The method of claim 90, wherein the disease condition is a predetermined subtype of a cancer.

104. The method of claim 73, the method further comprising:
(D) applying the plurality of sequence reads to a trained classifier thereby obtaining a classifier result, wherein the trained classifier result indicates whether the subject has a first cancer condition; and
(E) using the trained classifier result as a basis for diagnosis of the subject for the first cancer condition when the tumor fraction is between 0.003 and 1.0 and the trained classifier result indicates that the subject has the first cancer condition.

105. The method of claim 104, wherein the first cancer condition is a cancer.

106. The method of claim 105, wherein the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer or a combination thereof.

107. The method of claim 104, wherein the first cancer condition is a subtype of a cancer.

108. The method of claim 107, wherein the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, or gastric cancer.

109. The method of claim 104, wherein the first tumor fraction is between 0.003 and 1.0 and the first cancer condition is a tissue of origin of a cancer.

110. The method of claim 104, wherein the trained classifier is a neural network, a support vector machine, a decision tree, an unsupervised clustering model, a supervised clustering model, or a regression model.

111. A computing system, comprising:
one or more processors;
memory storing one or more programs to be executed by the one or more processors;
the one or more programs comprising instructions determining tumor fraction in cell-free nucleic acid of a liquid biological sample of a subject by a method comprising:
(A) obtaining a plurality of sequence reads in electronic form from the liquid biological sample of the subject, wherein the liquid biological sample comprises cell-free nucleic acid molecules;
(B) using the plurality of sequence reads to identify support for each variant in a variant set thereby determining an observed frequency of each variant in the first variant set; and
(C) deeming the observed frequency of the variant having the N^{th} highest allele frequency in the variant set to be the tumor fraction in cell-free nucleic acid of the liquid biological sample of the subject, wherein N is a positive integer other than one.

112. A non-transitory computer readable storage medium storing one or more programs for determining tumor fraction in cell-free nucleic acid of a liquid biological sample of a subject, the one or more programs configured for execution by a computer, the one or more programs comprising instructions for:
(A) obtaining a plurality of sequence reads in electronic form from the liquid biological sample of the subject, wherein the liquid biological sample comprises cell-free nucleic acid molecules;
(B) using the plurality of sequence reads to identify support for each variant in a variant set thereby determining an observed frequency of each variant in the first variant set; and
(C) deeming the observed frequency of the variant having the N^{th} highest allele frequency in the variant set to be the tumor fraction in cell-free nucleic acid of the liquid biological sample of the subject, wherein N is a positive integer other than one.
